(19) United States
(12) Patent Application Publication
(10) Pub. No.: US 2011/0160206 A1
(43) Pub. Date: Jun. 30, 2011

(54) TRI-SUBSTITUTED PYRIMIDINE COMPOUNDS AND THEIR USE AS PDE10 INHIBITORS

(76) Inventors: Eiji Kawanishi, Osaka (JP); Takehiko Matsumura, Osaka (JP)

(21) Appl. No.: 13/062,074

(22) PCT Filed: Sep. 3, 2009
(86) PCT No.: PCT/JP2009/065778

§ 371 Date: Mar. 3, 2011

(51) Int. Cl. A61K 31/5377; C07D 403/14; A61K 31/498; A61K 31/506; C07D 413/14; A61P 25/18; A61P 25/22; A61P 25/28

(52) U.S. Cl. 514/234.8; 544/295; 514/249; 544/326; 514/256; 544/116

(57) ABSTRACT

The present invention provides a tri-substituted pyrimidine compound having an excellent PDE10 inhibitory activity. The present invention relates to a tri-substituted pyrimidine compound represented by the following formula [I⁰] or a pharmaceutically acceptable salt thereof, a method for preparing the same, and use of said compound for PDE10 inhibitor, and a pharmaceutical composition comprising said compounds as an active ingredient: wherein: either one of X¹ and X² is N, and the other of X¹ and X² is CH; A is *-CH═CH—, *-C(Alk)=CH—, *-CH₂—CH₂— or *-O—CH₂— (* is a bond with R¹); Alk is a lower alkyl group; Ring B is an optionally substituted nitrogen-containing aliphatic heterocyclic group; R¹ is an optionally substituted quinoxalinyl or an optionally substituted quinolyl; Y⁰ is mono- or di-substituted amino group, or a pharmaceutically acceptable salt thereof.

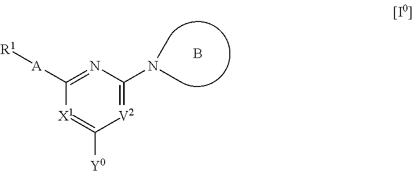

### TECHNICAL FIELD

The present invention relates to novel tri-substituted pyrimidine compounds having an excellent phosphodiesterase 10 (PDE10) inhibitory activity and useful as pharmaceuticals, and to processes for preparing such compounds and to their use.

### BACKGROUND ART

Cyclic nucleotide phosphodiesterase (hereinafter referred to as phosphodiesterase or PDE) is an enzyme that hydrolyses a phosphodiester bond in cyclic nucleotides such as cAMP (adenosine 3′,5′-cyclic monophosphate) or cGMP (guanosine 3′,5′-cyclic monophosphate), etc. as a substrate, to provide nucleotides such as 5′AMP (adenosine 5′-monophosphate) or 5′GMP (guanosine 5′-monophosphate), etc.

Cyclic nucleotides such as cAMP and cGMP are involved in the regulation of many functions within a living body as second messengers of intracellular signaling. Intracellular concentrations of cAMP and cGMP, which vary in response to extracellular signals, are regulated by a balance between enzymes involved in synthesis of cAMP and cGMP (adenylate cyclase and guanylate cyclase) and PDE involved in hydrolysis of such enzymes.

For PDE of mammals, many kinds of PDEs have been isolated and identified in mammals so far, and they have been classified into plural families in accordance with amino-acid sequence homology, biochemical properties, characterization by inhibitors and the like (Francis et al., Prog. Nucleic Acid Res., vol. 65, pp. 1-52, 2001).

Among such various families of PDEs of mammals, phosphodiesterase 10 (PDE10) [more specifically phosphodiesterase 10A (PDE10A)] recognizes both cAMP and cGMP as a substrate. It has been reported that PDE10 has a greater affinity for cAMP. Further, cDNAs of human, mouse and rat PDE10As have been isolated and identified. Furthermore, the existence of PDE10 proteins has been confirmed. (Fujishige et al., J. Biol. Chem., vol. 274, pp. 18438-18445, 1999; Kotera et al., Biochem. Biophys. Res. Commun., vol. 261, pp. 551-557, 1999; Soderling et al., Proc. Natl. Acad. Sci. USA, vol. 96, pp. 7071-7076, 1999; and Loughley et al., Gene, vol. 234, pp. 109-117, 1999).

Regarding PDE10 inhibitory compounds (PDE10 inhibitors), that is, compounds having inhibitory action on the enzyme activity of PDE10, the followings have been reported:

For example, in EP1250923 (Pfizer) and WO2005/082883 (Pfizer), papaverine and various aromatic heterocyclic compounds such as quinazoline and isoquinazoline compounds are disclosed as PDE10 inhibitors.

It also has been disclosed therein that PDE10 inhibitors are useful for the treatment or prophylaxis of diseases or conditions such as:

Psychotic disorder:
for example, schizophrenia, schizophreniform disorder,
delusional disorder, substance-induced psychotic disorder, personality disorder of the paranoid type, personality disorder of the schizoid type, etc;

Anxiety disorder:
for example, panic disorder, agoraphobia, specific phobia, social phobia, obsessive-compulsive disorder, post-traumatic stress disorder, acute stress disorder, generalized anxiety disorder, etc;

Movement disorder:
for example, Huntington's disease, dyskinesia associated with dopamine agonist therapy, Parkinson's disease, restless leg syndrome, etc;

Drug addiction:
for example, addiction to alcohol, amphetamine, cocaine, or opiate, etc;

Disorders comprising deficient cognition as a symptom:
for example, dementia (including Alzheimer's disease, multi-infarct dementia, etc), delirium, amnestic disorder, post-traumatic stress disorder, mental retardation, a learning disorder, attention deficit hyperactivity disorder (ADHD), age-related cognitive decline, etc; and

Mood disorder:
for example, major depressive disorder, dysthymic disorder, minor depressive disorder, bipolar disorder (including bipolar I disorder, bipolar II disorder), cyclothymic disorder, etc; or

Mood episode:
for example, major depressive episode, manic or mixed mood episode, hypomanic mood episode, etc.

Further, it also has been disclosed therein that PDE10 inhibitors are useful for the treatment or prophylaxis of neurodegenerative disorders, for example, Parkinson's disease, and Hungtington's disease, etc.

In the literature of Menniti et al. [Menniti et al., Curr. Opin. Investig. Drugs., 2007, 8(1):54-59], it is disclosed that PDE10 inhibitors have potential as antipsychotic agents along with potential to improve cognitive symptoms in schizophrenia.

WO2003/000693 (Bayer) discloses imidazotriazine compounds as PDE10 inhibitors. It also discloses that PDE10 inhibitors are useful for the treatment or prophylaxis of neurodegenerative disorders, especially for Parkinson's disease.

WO2003/014117 (Bayer) etc discloses various pyrroloisoquinoline compounds as PDE10 inhibitors. It also discloses that these compounds having inhibitory action on PDE10 activity show antiproliferative activity and are useful for treating cancer. Further, it discloses that those compounds are useful for treating conditions of pain and/or for lowering the temperature of the body in fever condition.

WO2005/12485 (Bayer) discloses that PDE10 inhibitors are useful for stimulating insulin release from pancreatic cells. Further, it is disclosed that PDE10 inhibitors are useful for the treatment or prophylaxis of diabetes and diseases related thereof:

for example, type 1 or type 2 diabetes, maturity-onset diabetes of the young (MODY), latent autoimmune diabetes adult (LADA), impaired glucose tolerance (IGT), impaired fasting glucose (IGF), gestational diabetes, metabolic syndrome X, etc.

See also WO2005/120514 (Pfizer), which discloses PDE10 inhibitors that are said to be useful to decrease body weight and/or body fat in the treatment of obese patients. Further, it is disclosed therein that those PDE10 inhibitors are useful for treatment of non-insulin dependent diabetes (NIDDM), metabolic syndrome and glucose intolerance etc.

In addition, certain pyrimidine compounds are known. See for example WO2002/38551 (Roche) which discloses tri-substituted pyrimidine compounds having an activity as Neuropeptide Y receptor ligands.

### DISCLOSURE OF THE INVENTION

The present invention provides novel compounds having an excellent PDE10 inhibitory activity, processes for preparing such compounds, use of the compounds, and pharmaceutical compositions comprising said compounds, and the like.

The present inventors have been studied and as a result, have been found that certain tri-substituted pyrimidine compounds have excellent PDE 10 inhibitory activity.

Namely, the present invention relates to a tri-substituted pyrimidine compound represented by formula [I⁰]:

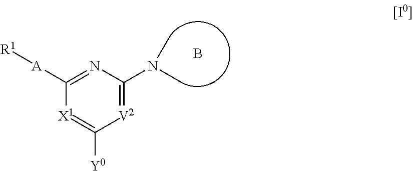

wherein:

either one of X¹ and X² is N, and the other of X¹ and X² is CH;

A is *-CH═CH—, *-C(Alk)=CH—, *-CH₂—CH₂— or *-O—CH₂— (* is a bond with R¹);

Alk is a lower alkyl group;

Ring B is an optionally substituted nitrogen-containing aliphatic heterocyclic group;

R¹ is an optionally substituted quinoxalinyl or an optionally substituted quinolyl;

Y⁰ is a mono- or di-substituted amino group,

or a pharmaceutically acceptable salt thereof.

Also, in one of the preferred embodiments of the invention, the present invention relates to a tri-substituted pyrimidine compound represented by formula [I]:

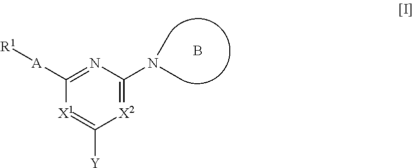

wherein:

either one of X¹ and X² is N, and the other of X¹ and X² is CH;

A is *-CH═CH—, *-C(Alk)=CH—, *-CH₂—CH₂— or *-O—CH₂— (* is a bond with R¹);

Alk is a lower alkyl group;

Ring B is an optionally substituted nitrogen-containing aliphatic heterocyclic group;

R¹ is an optionally substituted quinoxalinyl or an optionally substituted quinolyl;

Y is a substituted amino group of formula:

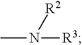

R² is a group selected from the group consisting of the following formula (1), (2) and (3); or R² and R³, together with the nitrogen atom to which they are attached, form a morpholino group, or a piperidino group substituted on 4-position by lower alkoxy;

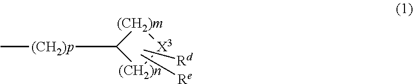

wherein:
X³ is —O—, —S— or —SO₂—;
m and n are each independently 0, 1, 2, 3 or 4, and m+n is 2, 3, 4 or 5;
p is 0, 1, 2, 3 or 4; and
R^{d} and R^{e} are the same or different and each independently are hydrogen, lower alkyl or halogen;

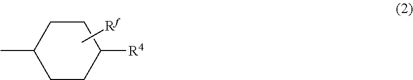

wherein:
R⁴ is a group selected from the group consisting of hydroxy, lower alkoxy, lower cycloalkyloxy, hydroxy-substituted lower alkyl, lower alkoxy-substituted lower alkyl and lower cycloalkyloxy-substituted lower alkyl; and
R^{f} is hydrogen, lower alkyl, lower cycloalkyl, or halogen; and

—(CH₂)_{q}—O—R⁵ (3)

wherein:

R⁵ is hydrogen, lower alkyl or lower cycloalkyl; and

q is 1, 2, 3 or 4;

R³ is a group selected from the group consisting of hydrogen, lower alkyl, lower cycloalkyl, lower alkoxy-substituted lower alkyl and lower cycloalkyloxy-substituted lower alkyl;

or R³ and R², together with the nitrogen atom to which they are attached, form a morpholino group, or a piperidino group substituted on the 4-position by lower alkoxy, or a pharmaceutically acceptable salt thereof.

Also, the present invention relates to a method for treating or preventing a disease comprising administering to a patient in need thereof an effective amount of the tri-substituted pyrimidine compound represented by formula [I⁰] or [I] or a pharmaceutically acceptable salt thereof.

Further, the present invention relates to a pharmaceutical composition comprising said compound of formula [I⁰] or [I] or a pharmaceutically acceptable salt thereof as an active ingredient, as well as to use of said compound for the manufacture of a medicament.

Furthermore, the present invention relates to said compound of formula [I⁰] or [I] or a pharmaceutically acceptable salt thereof, and to a process for preparing said compound.

The compounds of formula [I⁰] or [I] or a pharmaceutically acceptable salt thereof according to the present invention has an excellent PDE10 inhibitory activity (that is, inhibitory activity on the enzyme activity of phosphodiesterase 10).

The compounds of the present invention and a pharmaceutical composition containing thereof as an active ingredient are useful for the treatment or prophylaxis of a disease or condition which is expected to be ameliorated by inhibition of PDE10 activity (that is, inhibition on the enzyme activity of phosphodiesterase 10) [for example, schizophrenia, anxiety disorder, drug addiction, a disease comprising as a symptom a deficiency in cognition, mood disorder and mood episode, etc].

### DETAILED DESCRIPTION OF THE INVENTION

Geometric isomers (E isomer or Z isomer) of formula [I⁰] or [I] may be exist due to a double bond in the molecule, for example, when a compound is of formula [I⁰] or [I] wherein A is *-CH═CH— or *-C(Alk)=CH—, etc. In the present invention, both geometric isomers and a mixture thereof are encompassed within a scope of the present invention.

In the present invention, the following terms have the following meanings, unless otherwise indicated.

Lower alkyl, lower alkylthio, lower alkyl sulfonyl, and lower alkyl amino include straight or branched group having 1 to 6 carbon atom(s) (C₁₋₆), preferably 1 to 4 carbon atom(s) (C₁₋₄).

Lower cycloalkyl includes cyclic group having 3 to 8 carbon atoms (C₃₋₈), preferably 3 to 6 carbon atoms (C₃₋₆). Also included in the lower cycloalkyl are ones having 1 to 2 lower alkyl substituent(s) on their cyclic moiety.

Lower alkoxy includes ones having 1 to 6 carbon atom(s) (C₁₋₆), preferably 1 to 4 carbon atom(s) (C₁₋₄). Included in the lower alkoxy are any of lower alkyl-O— or lower cycloalkyl-O—.

Lower alkanoyl and lower alkanoylamino include ones having 2 to 7 carbon atoms (C₂₋₇), preferably 2 to 5 carbon atoms (C₂₋₅). Included in lower alkanoyl are any of lower alkyl-C(O)— or lower cycloalkyl-C(O)—.

Lower alkylene includes straight or branched group having 1 to 6 carbon atom(s) (C₁₋₆), preferably 1 to 4 carbon atom(s) (C₁₋₄).

Lower alkenyl and lower alkenylene include ones having 2 to 7 carbon atoms (C₂₋₇), preferably 2 to 5 carbon atoms (C₂₋₅) and at least one double bond.

Lower cycloalkenyl includes a cyclic group having 3 to 8 carbon atoms (C₃₋₈), preferably 3 to 6 carbon atoms (C₃₋₆). Also included in lower cycloalkenyl are ones having 1 to 2 lower alkyl substituent(s) on their cyclic moiety.

Halogen means fluorine, chlorine, bromine or iodine. Halo means fluoro, chloro, bromo or iodo.

Included in the optionally substituted amino groups are unsubstituted amino groups, mono- or di-substituted acyclic amino groups, and, also included are cyclic amino groups, for example, 1-pyrrolidinyl, 1-piperidyl, 1-piperazinyl, 4-morpholinyl, etc.

When a compound of formula [I⁰] or [I] is one wherein A is *-CH═CH— or *-C(Alk)=CH—, both geometric isomers (E isomer and Z isomer) may be exist and both isomers are encompassed within a scope of the present invention. Among them, the E isomer is preferred.

In compound of formula [I⁰] or [I], “Alk” may include methyl, ethyl, propyl, butyl and the like. Among them, methyl is more preferred.

Suitable examples of “an optionally substituted quinoxalinyl” represented by R¹ include “optionally substituted quinoxalin-2-yl”.

Suitable examples of “an optionally substituted quinolyl” include “optionally substituted quinolin-2-yl”.

Substituent(s) in “an optionally substituted quinoxalinyl” or “an optionally substituted quinolyl” may be 1 or more, for example, 1 to 3, which may be same or different.

Examples of such substituents include:

halogen; hydroxy; optionally substituted lower alkyl; optionally substituted lower cycloalkyl; optionally substituted lower alkoxy; and optionally substituted amino group; etc.

Among them, the following are of interest:

halogen; hydroxy; nitro group;
lower alkyl which may be substituted by halogen etc;
lower cycloalkyl which may be substituted by halogen etc; lower alkoxy which may be substituted by halogen etc; and
amino group which may be mono- or di-substituted by the same or different substituent(s) selected from the group consisting of lower alkyl and lower cycloalkyl.

More specific examples of “an optionally substituted quinoxalinyl or an optionally substituted quinolyl” represented by R¹ include a group represented by formula [X]:

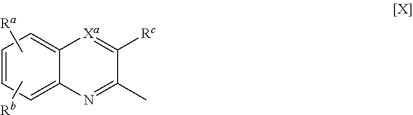

wherein:

X^{a} is N or CH;

R^{a}, R^{b} and R^{c} each independently are selected from the group consisting of hydrogen, halogen, hydroxy, lower alkyl, lower cycloalkyl, halo-lower alkyl, lower alkoxy, halo-lower alkoxy, nitro group, amino group, and amino group mono- or di-substituted by the same or different substituent(s) selected from the group consisting of lower alkyl and lower cycloalkyl.

The nitrogen-containing aliphatic heterocycle moiety in the “optionally substituted nitrogen-containing aliphatic heterocyclic group” represented by Ring B includes saturated or unsaturated, monocyclic or bicyclic aliphatic heterocycle containing one nitrogen atom and 0 or more hetero atom(s) selected from the group consisting of nitrogen, oxygen and sulfur.

The monocyclic ones in the above nitrogen-containing aliphatic heterocycle includes saturated or unsaturated 5 to 7-membered aliphatic heterocycle containing one nitrogen and 0 to 3 hetero atom(s) selected from the group consisting of nitrogen, oxygen and sulfur.

The bicyclic ones in the above nitrogen-containing aliphatic heterocycle includes aliphatic heterocycle in which two saturated or unsaturated 5 to 7-membered ring are fused and in which are contained one nitrogen atom and 0 to 5 hetero atom(s) selected from nitrogen, oxygen and sulfur.

Specific examples include 1-pyrrolidinyl, 1-imidazolidinyl, 1-pyrazolidinyl, 1-piperidyl, 1-piperazinyl, 4-morpholinyl, 4-thiomorpholinyl, 1-perhydroazepinyl, or a monocyclic group in which a part thereof is unsaturated.

Among these rings, preferred are 1-pyrrolidinyl, 1-imidazolidinyl, 1-piperidyl, 1-piperazinyl, or 4-morpholinyl, and particularly preferred is 1-pyrrolidinyl.

Examples of substituents on said nitrogen-containing aliphatic heterocyclic group include: oxo; hydroxy; lower alkyl; lower alkoxy; substituted or unsubstituted amino. The substituent(s) may be 1 to 3 or more and each may be the same or different.

The “mono- or di-substituted amino” group represented by Y⁰ includes an acyclic amino group substituted by 1 or 2 substituent(s) which may be the same or different.

Examples of such substituents include:

an optionally substituted lower alkyl group, which may have 1 to 3 substituent(s) which may be the same or different and selected from the group consisting of hydroxy, lower alkyl, and lower alkoxy, etc;
an optionally substituted lower cycloalkyl, which may have 1 to 3 substituent(s) which may be the same or different and selected from the group consisting of hydroxy, lower alkyl, lower alkoxy, hydroxy-lower alkyl and lower alkoxy-lower alkyl, etc; and an optionally substituted 4 to 7-membered (preferably 5 to 6-membered) aliphatic monocyclic heterocyclic group, such as oxolanyl, tetrahydropyranyl and thiolanyl, each of which may have 1 to 3 substituent(s) which may be the same or different and are selected from the group consisting of oxo and lower alkyl, etc.

The di-substituted amino group represented by Y⁰ includes an optionally substituted cyclic amino. Examples of the cyclic amino include 1-pyrrolidinyl, 1-piperidyl, 1-piperazinyl, 4-morpholinyl and the like. The cyclic amino may be substituted on its ring moiety by 1 to 3 substituent(s) which may be the same or different and selected from the group consisting of oxo, hydroxy, lower alkyl and lower alkoxy, etc.

In the group (1) of R² represented by:

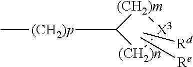

m+n is preferably 3 or 4, and p is preferably 0 or 1.

One aspect of the present invention includes those compounds of formula [I] wherein “A” is *-CH═CH— or *-C(Alk)=CH—. In this embodiment of the invention, the E isomeric form of the double bond in “A” is preferred.

Another aspect of the present invention includes those compounds of formula [I] wherein R¹ is a group represented by formula [X]:

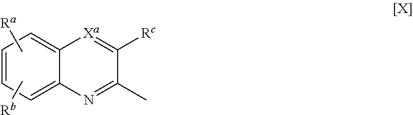

wherein the symbols are as defined above. The preferred embodiments of [X] are ones wherein X^{a} is N.

Another aspect of the invention includes those compounds of formula [I] wherein R² is a group represented by the following formula:

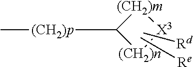

wherein the symbols are as defined above.

Another aspect of the invention includes those compounds of formula [I] wherein R² is a group represented by the following formula:

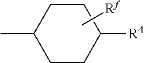

wherein the symbols are as defined above.

Another aspect of the invention includes those compounds of formula [I] wherein A is *-CH═CH—, *-C(Alk)=CH— or *-CH₂—CH₂—.

Another aspect of the invention includes those compounds of formula [I] wherein A is *-CH═CH—.

Another aspect of the invention includes those compounds of formula [I] wherein X¹ is N, X² is CH, and A is *-CH═CH—. Another aspect of the invention includes those compounds of formula [I]wherein A is *-O—CH₂—.

Another aspect of the invention includes a free form of each compound disclosed in the Examples or a pharmaceutically acceptable salt thereof (such as hydrochloride, sulfate, nitrate, phosphate, hydrobromate, acetate, fumarate, oxalate, citrate, methanesulfonate, benzenesulfonate, p-toluenesulfonate or maleate thereof).

Another aspect of the invention includes a compound selected from
N,N-dimethyl-3-{(E)-2-[4-pyrrolidin-1-yl-6-(tetrahydro-2H-pyran-4-ylamino)pyrimidin-2-yl]vinyl}quinoxalin-2-amine;
3-((E)-2-{4-[(2-methoxyethyl)amino]-6-pyrrolidin-1-ylpyrimidin-2-yl}vinyl)-N,N-dimethylquinoxalin-2-amine;
3-[(E)-2-(4-{[(3R)-1,1-dioxidotetrahydro-3-thienyl]amino}-6-pyrrolidin-1-ylpyrimidin-2-yl)vinyl]-N,N-dimethylquinoxalin-2-amine;
N-cyclopropyl-N-methyl-3-{(E)-2-[4-pyrrolidin-1-yl-6-(tetrahydro-2H-pyran-4-ylamino)pyrimidin-2-yl]vinyl}quinoxalin-2-amine;
trans-1-methyl-4-({2-[(E)-2-(3-methylquinoxalin-2-yl)vinyl]-6-pyrrolidin-1-ylpyrimidin-4-yl}amino)cyclohexanol;

[trans-4-({2-[(E)-2-(3-methylquinoxalin-2-yl)vinyl]-6-pyrrolidin-1-ylpyrimidin-4-yl}amino)cyclohexyl]methanol;
6-pyrrolidin-1-yl-N-[(3R)-tetrahydrofuran-3-yl]-2-[(E)-2-(3,6,7-trimethylquinoxalin-2-yl)vinyl]pyrimidin-4-amine;
2-[(E)-2-(6-fluoro-3-methylquinoxalin-2-yl)vinyl]-N-(trans-4-methoxycyclohexyl)-6-pyrrolidin-1-ylpyrimidin-4-amine;
2-[(E)-2-(7-fluoro-3-methylquinoxalin-2-yl)vinyl]-6-pyrrolidin-1-yl-N-(tetrahydro-2H-pyran-4-yl)pyrimidin-4-amine;
trans-4-({2-[(E)-2-(3,7-dimethylquinoxalin-2-yl)vinyl]-6-pyrrolidin-1-ylpyrimidin-4-yl}amino)-1-methylcyclohexanol;
N-[(3R)-1,1-dioxidotetrahydro-3-thienyl]-2-{(E)-2-[3-methyl-7-(trifluoromethyl)quinoxalin-2-yl]vinyl}-6-pyrrolidin-1-ylpyrimidin-4-amine;
2-[(E)-2-(7-methoxy-3-methylquinoxalin-2-yl)vinyl]-6-pyrrolidin-1-yl-N-(tetrahydro-2H-pyran-4-yl)pyrimidin-4-amine;
trans-4-[(2-{(E)-2-[3-methyl-7-(trifluoromethoxy)quinoxalin-2-yl]vinyl}-6-pyrrolidin-1-ylpyrimidin-4-yl)amino]cyclohexanol;
2-[(E)-2-(3-methylquinolin-2-yl)vinyl]-6-pyrrolidin-1-yl-N-(tetrahydro-2H-pyran-4-yl)pyrimidin-4-amine;
N-[(3R)-1,1-dioxidotetrahydro-3-thienyl]-2-[(E)-2-(3-methylquinolin-2-yl)vinyl]-6-pyrrolidin-1-ylpyrimidin-4-amine;
3-{(E)-2-[4-pyrrolidin-1-yl-6-(tetrahydro-2H-pyran-4-ylamino)pyrimidin-2-yl]vinyl}quinoxalin-2-ol;
N,N-dimethyl-3-[(E)-2-(4-morpholin-4-yl-6-pyrrolidin-1-ylpyrimidin-2-yl)vinyl]quinoxalin-2-amine;
3-((E)-2-{4-[cyclopropyl(tetrahydro-2H-pyran-4-yl)amino]-6-pyrrolidin-1-ylpyrimidin-2-yl}vinyl)-N,N-dimethylquinoxalin-2-amine;
N-cyclopropyl-N-methyl-3-((E)-2-{4-[methyl(tetrahydro-2H-pyran-4-yl)amino]-6-pyrrolidin-1-ylpyrimidin-2-yl}vinyl)quinoxalin-2-amine;
N-(trans-4-methoxycyclohexyl)-2-{2-[3-methyl-7-(trifluoromethyl)quinoxalin-2-yl]ethyl}-6-pyrrolidin-1-ylpyrimidin-4-amine;
N-methyl-2-[(3-methylquinoxalin-2-yl)oxy]methyl)-6-pyrrolidin-1-yl-N-(tetrahydro-2H-pyran-4-yl)pyrimidin-4-amine; and
6-{[(3-methylquinoxalin-2-yl)oxy]methyl}-2-pyrrolidin-1-yl-N-(tetrahydro-2H-pyran-4-yl)pyrimidin-4-amine;
   or a pharmaceutically acceptable salt thereof (such as hydrochloride, sulfate, nitrate, phosphate, hydrobromate, acetate, fumarate, oxalate, citrate, methanesulfonate, benzenesulfonate, p-toluenesulfonate or maleate thereof).

The compounds of formula [I⁰] or [I] of the present invention may be a free form (free base or free acid) or a pharmaceutically acceptable salt thereof. Examples of the pharmaceutically acceptable salts include inorganic acid salts such as the hydrochloride, sulfate, nitrate, phosphate or hydrobromate, and organic acid salts such as the acetate, fumarate, oxalate, citrate, methanesulfonate, benzenesulfonate, p-toluenesulfonate or maleate, and the like. Further, when the compounds of the present invention contain substituent(s) such as carboxyl group, the pharmaceutically acceptable salts thereof may include salts with bases such as alkali metal salts such as sodium salts and potassium salts or alkaline earth metal salts such as calcium salts.

The compounds of formula [I⁰] or [I] or a salt thereof encompass any of intramolecular salts, adducts, solvates or hydrates thereof.

The compounds of formula [I] can be prepared by a number of methods such as, but not limited to, the following:

### Scheme A1, Scheme A2, Scheme B, Scheme C1 and Scheme C2.

The compounds of formula [I⁰] can also be prepared in the same manner as set out for preparing the compound of formula [I] but using the appropriate corresponding starting materials and reactants, solvents, etc.

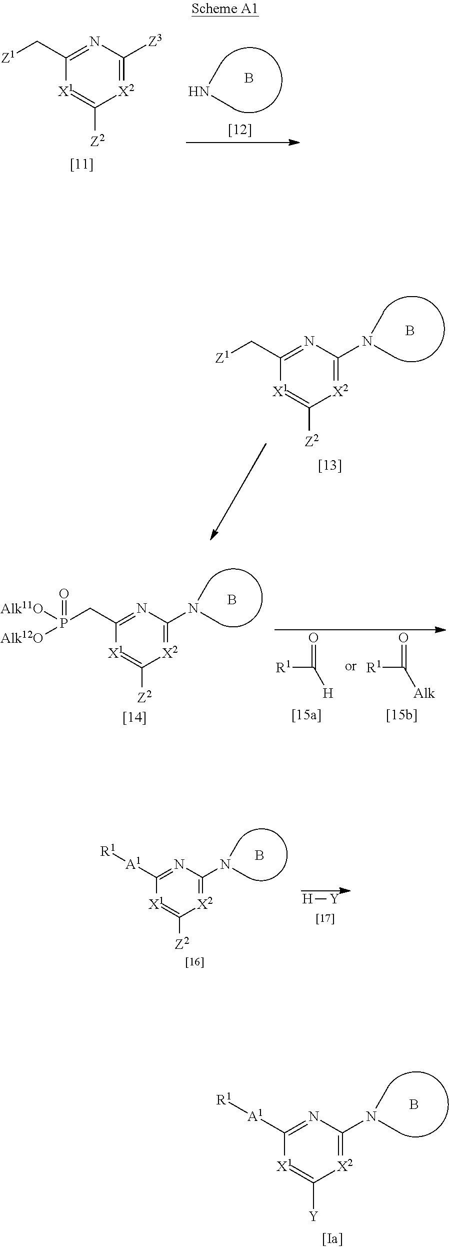

Compounds of formula [I] wherein A is *-CH═CH— or *-C(Alk)=CH—, represented by formula [Ia]:

wherein A¹ is *-CH═CH— or *-C(Alk)=CH—
(* is a bond with R¹), and the other symbols have the same meaning as defined above,
   can be prepared by the following manners.

First, a compound represented by formula [11]:
wherein Z′, Z² and Z³ independently are a reactive residue, and the other symbols have the same meaning as defined above,
   is reacted with a compound represented by formula [12]:
wherein the symbols have the same meaning as defined above,
   or a salt thereof to provide a compound represented by formula [13]:
wherein the symbols have the same meaning as defined above.
   The compound of formula [13] is reacted with phosphite esters such as dimethyl phosphite, diethyl phosphite, diisopropyl phosphite, diphenyl phosphite, di(2,2,2-trifluoroethyl)phosphite, trimethyl phosphite, triethyl phosphite, triisopropyl phosphite, tri(2,2,2-trifluoroethyl)phosphite, etc,
   to provide a compound represented by formula [14]:
wherein Alk¹¹ and Alk¹² are the same or different alkyl group, and the other symbols have the same meaning as defined above.
   The compound of formula [14] is reacted with a compound represented by formula [15a] or [15b]:

wherein the symbols have the same meaning as defined above,

to provide a compound represented by formula [16]:

wherein the symbols have the same meaning as defined above.

The compound of formula [16] is reacted with a compound represented by formula [17]:

wherein the symbols have the same meaning as defined above,

or a salt thereof to provide a compound of formula [Ia] which is optionally converted to a pharmaceutically acceptable salt thereof.

The reactive residues Z¹, Z² and Z³ suitably employed in the reaction include those conventionally used such as halogen, lower alkylsulfonyloxy group and arylsulfonyloxy group. Preferably the group is halogen.

Preferred salts of the compounds of formulae [12] and [17] are, for example, a salt formed with an inorganic acid such as hydrochloric acid and sulfuric acid, or a salt formed with inorganic base such as alkali metal base and alkali earth metal base.

The reactions in Scheme A1 can be carried out as described below.

The reaction of a compound of formula [11] with a compound of formula [12] or a salt thereof can be carried out in a suitable solvent in the presence or absence of a base. Such bases include organic bases, for example, triethylamine, diisopropylethylamine, N-methylmorpholine, pyridine, dimethylaniline, dimethylaminopyridine and the like; or inorganic bases, for example, an alkali metal hydride such as sodium hydride, an alkali metal carbonate such as sodium carbonate and potassium carbonate, an alkali metal amide such as sodium amide and lithium amide, an alkali metal such as sodium, an alkali metal hydroxide such as sodium hydroxide and potassium hydroxide, and the like.

This reaction suitably proceeds at −78° C. to 200° C., particularly at 0° C. to 100° C.

The solvent employed may be any solvent which does not have a negative impact on the reaction. Examples include acetonitrile, methanol, ethanol, isopropyl alcohol, n-propyl alcohol, tert-butyl alcohol, acetone, N,N-dimethylformamide, dimethylsulfoxide, tetrahydrofuran, diethyl ether, dioxane, ethyl acetate, toluene, methylene chloride, dichloroethane, chloroform, N,N-dimethylacetamide, 1,3-dimethyl-2-imidazolidinone, 1-methyl-2-pyrrolidinone, 1,2-dimethoxyethane, xylene, or a combination thereof.

The reaction of a compound of formula [13] with phosphite esters can be carried out in a suitable solvent in the presence or absence of a base.

If a base is used, it can be inorganic bases such as an alkali metal hydride such as sodium hydride, an alkali metal carbonate such as sodium carbonate and potassium carbonate, an alkali metal amide such as sodium amide and lithium amide, an alkali metal alkoxide such as lithium tert-butoxide, sodium tert-butoxide, potassium tert-butoxide, sodium methoxide and sodium ethoxide, an alkali metal such as sodium, or an alkali metal hydroxide such as sodium hydroxide and potassium hydroxide, and the like. Organic bases such as triethylamine, diisopropylethylamine, morpholine, N-methylmorpholine, pyridine, piperidine, dimethylaniline, dimethylaminopyridine and the like can also be used.

This reaction suitably proceeds at −78° C. to 100° C., particularly at 0° C. to room temperature.

The solvent employed in this step may be any solvent which does not have a negative impact on the reaction. Examples include acetonitrile, methanol, ethanol, isopropyl alcohol, n-propyl alcohol, tert-butyl alcohol, N,N-dimethylformamide, dimethylsulfoxide, tetrahydrofuran, diethyl ether, dioxane, ethyl acetate, toluene, methylene chloride, dichloroethane, chloroform, N,N-dimethylacetamide, 1,3-dimethyl-2-imidazolidine, 1-methyl-2-pyrrolidinone, 1,2-dimethoxyethane, xylene, or a combination thereof.

The reaction of a compound of formula [14] with a compound of formula [15a] or [15b] can be carried out in a suitable solvent in the presence or absence of a base. If a base is used, it may be selected from the same bases as those employed in the reaction in the preceeding step where a compound of formula [13] is treated with phosphite esters.

This reaction suitably proceeds at −78° C. to 100° C., particularly at −40° C. to 60° C.

The solvent employed in this step may be any solvent which does not have a negative impact on the reaction. Examples include the same solvents as those employed in the preceeding step where a compound of formula [13] is treated with phosphite esters.

The reaction of a compound of formula [16] with a compound of formula [17] can be carried out in a suitable solvent in the presence of a base or a catalyst.

If a base is used, it may be an inorganic base such as an alkali metal hydride such as sodium hydride, an alkali metal carbonate such as sodium carbonate and potassium carbonate, an alkali metal amide such as sodium amide and lithium amide, an alkali metal alkoxide such as sodium methoxide and sodium tert-butoxide, an alkali metal such as sodium, an alkali metal hydroxide such as sodium hydroxide and potassium hydroxide, or an alkyl alkali metal such as n-butyllithium, and the like. Or it may be an organic base such as triethylamine, diisopropylethylamine, morpholine, N-methylmorpholine, pyridine, dimethylaminopyridine, and the like.

If a catalyst is used, it may be palladium catalyst such as dichlorobis(triphenylphosphine)palladium, palladium acetate, palladium chloride, tetrakis(triphenylphosphine)palladium, bis(tri-t-butylphosphine)palladium, and the like; or copper iodide.

Further, for facilitating the reaction, phosphorus compounds such as triphenylphosphine, 2-dicyclohexylphosphino-2′,4′,6′-triisopropylbiphenyl, 2-dicyclohexylphosphino-2′-(N,N-dimethylamino)biphenyl, and 2,2′-bis(diphenylphosphino)-1,1″-binaphthyl, etc. may be added.

This reaction suitably proceeds at 0° C. to 200° C., particularly at room temperature to 110° C.

The solvent used may be any solvent which does not have a negative impact on the reaction. Examples include acetonitrile, methanol, ethanol, isopropyl alcohol, n-propyl alcohol, tert-butyl alcohol, acetone, N,N-dimethylformamide, dimethylsulfoxide, tetrahydrofuran, diethyl ether, dioxane, ethyl acetate, toluene, methylene chloride, dichloroethane, chloroform, N,N-dimethylacetamide, 1,3-dimethyl-2-imidazolidinone, 1-methyl-2-pyrrolidinone, 1,2-dimethoxyethane, xylene, N-methylpyrrolidone or a combination thereof

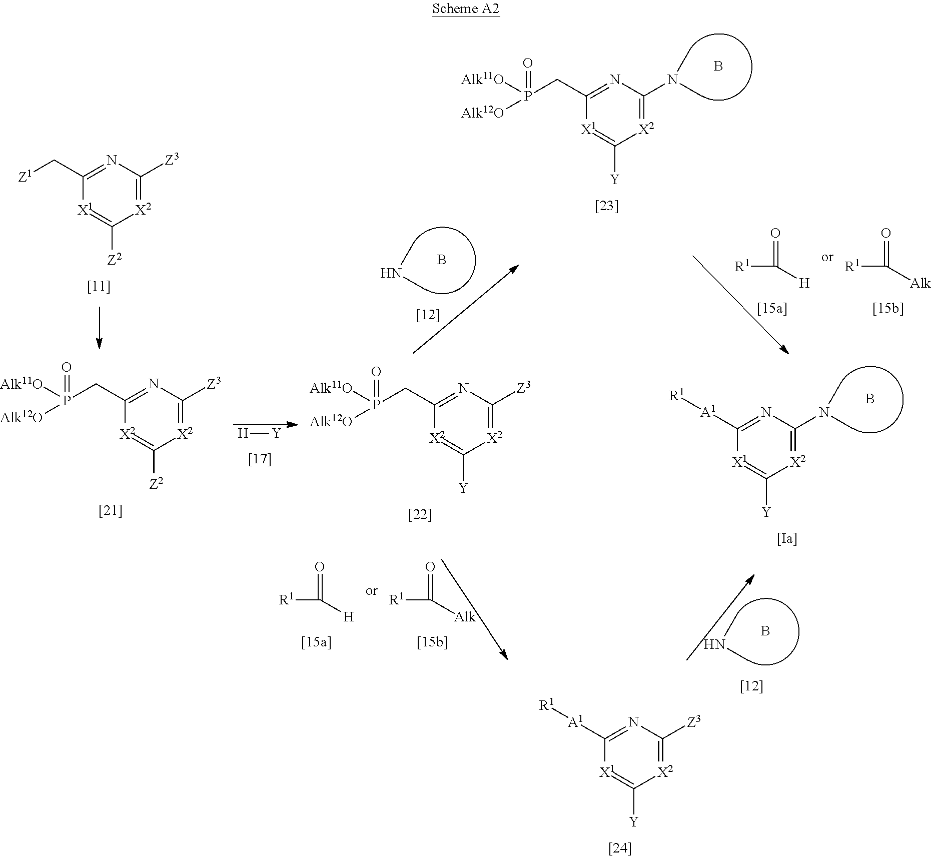

The compounds of formula [Ia] can be prepared by the following manner.

First, a compound represented by formula [11]:

wherein the symbols have the same meaning as defined above,

is reacted with phosphite esters (diethyl phosphite, dimethyl phosphite, etc.) to provide a compound represented by formula [21]:

wherein the symbols have the same meaning as defined above.

Then, a compound of formula [21] is reacted with a compound represented by formula [17]:

wherein the symbols have the same meaning as defined above,

or a salt thereof to provide a compound represented by formula [22]:

wherein the symbols have the same meaning as defined above.

A compound of formula [22] is reacted with a compound represented by formula [12]:

wherein the symbols have the same meaning as defined above,

or a salt thereof to provide a compound represented by formula [23]:

wherein the symbols have the same meaning as defined above.

A compound of formula [23] is then reacted with a compound represented by formula [15a] or [15b]:

wherein the symbols have the same meaning as defined above,

to provide a compound of formula [Ia] which is optionally converted to a pharmaceutically acceptable salt thereof.

Alternatively, a compound of formula [22] is reacted with a compound of formula [15a] or [15b] to provide a compound represented by formula [24]:

wherein the symbols have the same meaning as defined above.

Then, a compound of formula [24] is reacted with a compound of formula [12] or a salt thereof to provide a compound of formula [Ia] which is optionally converted to a pharmaceutically acceptable salt thereof.

The reactions in Scheme A2 can be carried out as described below.

The reaction of a compound of formula [11] with phosphite esters can be carried out in the same manner as described above in Scheme A1 for reacting a compound of formula [13] with phosphite esters.

The reaction of a compound of formula [21] with a compound of formula [17] or a salt thereof can be carried out in the same manner as described above in Scheme A1 for reacting a compound of formula [16] with a compound of formula [17] or a salt thereof.

Reacting a compound of formula [22] with the compound [12] or a salt thereof can be carried out in the same manner as described above in Scheme A1 for reacting a compound of formula [11] with a compound of formula [12] or a salt thereof.

The reaction of a compound of formula [23] with a compound of formula [15a] or [15b] can be carried out in the same manner as described above in Scheme A1 for reacting a compound of formula [14] with a compound of formula [15a] or [15b].

The reaction of a compound of formula [22] with a compound of formula [15a] or [15b] can be carried out in the same manner as described above in Scheme A1 for reacting a compound of formula [14] with a compound of formula [15a] or [15b].

The reaction of a compound of formula [24] with a compound of formula [12] or a salt thereof can be carried out in the same manner as described above in Scheme A1 for reacting a compound of formula [11] with a compound of formula [12] or a salt thereof.

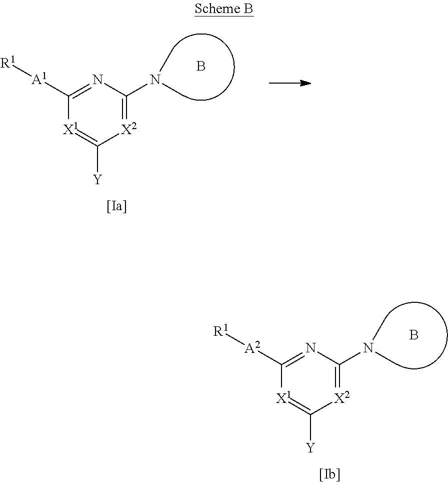

Compounds of formula [I] wherein A is *-CH₂—CH₂—, represented by formula [Ib]:
wherein A² is *-CH₂—CH₂— (* is a bond with R¹), and the other symbols have the same meaning as defined above,
   can be prepared as follows.

A compound of formula [Ia] can be reduced (hydrogenated) to provide a compound of formula [Ib] which is optionally converted to a pharmaceutically acceptable salt thereof.

The reduction (hydrogenation) reaction in Scheme B can be carried out by catalytic reduction process in a suitable solvent in the presence of a catalyst.

Such catalyst may be platinum oxide, Raney nickel, palladium carbon, palladium hydroxide and the like.

This reaction suitably proceeds at 0° C. to 100° C., particularly at room temperature to 50° C.

The solvent may be any one which does not have a negative impact on the reaction. Examples include acetonitrile, methanol, ethanol, isopropyl alcohol, n-propyl alcohol, tert-butyl alcohol, acetone, N,N-dimethylformamide, dimethylsulfoxide, tetrahydrofuran, diethyl ether, dioxane, ethyl acetate, toluene, methylene chloride, dichloroethane, chloroform, N,N-dimethylacetamide, 1,3-dimethyl-2-imidazolidinone, 1-methyl-2-pyrrolidinone, 1,2-dimethoxyethane, xylene, or a combination thereof

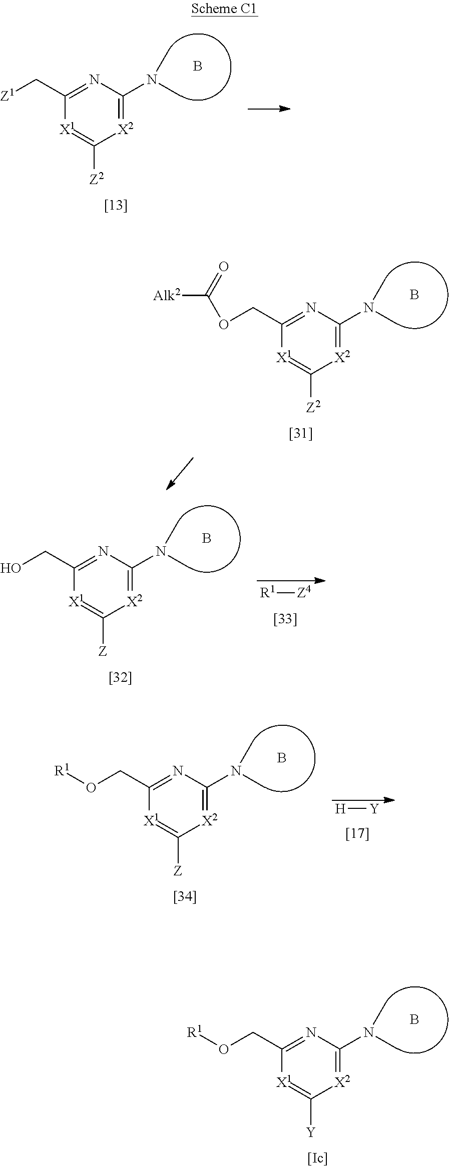

Compounds of formula [I] wherein A is *-O—CH₂—, represented by formula [Ic]:

wherein the symbols have the same meaning as defined above, can be prepared as follows.

First, a compound represented by formula [13]:

wherein the symbols have the same meaning as defined above,

is reacted with a carboxylic acid of formula Alk²-COOH:

wherein Alk² is a lower alkyl,

or a salt thereof to provide a compound represented by formula [31]:

wherein the symbols have the same meaning as defined above.

A compound of formula [31] is hydrolyzed to provide a compound represented by formula [32]:

wherein the symbols have the same meaning as defined above.

A compound of formula [32] is then reacted with a compound represented by formula [33]:
wherein Z⁴ is a reactive residue, and the other symbols have the same meaning as defined above,
   to provide a compound represented by formula [34]:

wherein the symbols have the same meaning as defined above.

A compound of formula [34] is reacted with a compound represented by formula [17]:

wherein the symbols have the same meaning as defined above, or a salt thereof to provide a compound of formula [Ic] which may be converted to a pharmaceutically acceptable salt thereof.

Reactive residue Z⁴ suitably employed in the reaction include those conventionally used such as halogen, lower alkylsulfonyloxy group and arylsulfonyloxy group. Preferably the group is halogen.

The reactions in Scheme C1 can be carried out as described below.

The reaction of a compound of formula [13] with a carboxylic acid of formula Alk²-COOH or a salt thereof can be carried out in a suitable solvent in the presence or absence of inorganic base or quaternary ammonium salt.

Such inorganic base or quaternary ammonium salt may include sodium iodide, tetrabutylammonium iodide and the like.

This reaction suitably proceeds at −20° C. to 100° C., particularly at 0° C. to room temperature.

The solvent employed may be any solvent which does not have a negative impact on the reaction. Examples include acetonitrile, methanol, ethanol, isopropyl alcohol, n-propyl alcohol, tert-butyl alcohol, N,N-dimethylformamide, dimethylsulfoxide, tetrahydrofuran, diethyl ether, dioxane, ethyl acetate, toluene, methylene chloride, dichloroethane, chloroform, N,N-dimethylacetamide, 1,3-dimethyl-2-imidazolidine, 1-methyl-2-pyrrolidinone, 1,2-dimethoxyethane, xylene, etc. or a combination thereof.

The hydrolysis reaction of a compound of formula [31] can be carried out in a suitable solvent in the presence or absence of a base.

Such base may include an organic base such as triethylamine, diisopropylethylamine, N-methylmorpholine, pyridine, dimethylaniline, dimethylaminopyridine and the like, or an inorganic base such as an alkali metal hydride such as sodium hydride, an alkali metal carbonate such as sodium carbonate and potassium carbonate, an alkali metal amide such as sodium amide and lithium amide, an alkali metal such as sodium, or an alkali metal hydroxide such as sodium hydroxide and potassium hydroxide.

This reaction suitably proceeds at −20° C. to 100° C., particularly at 0° C. to room temperature.

The solvent may be any solvent which does not have a negative impact on the reaction. Examples include acetonitrile, methanol, ethanol, isopropyl alcohol, n-propyl alcohol, tert-butyl alcohol, N,N-dimethylformamide, dimethylsulfoxide, tetrahydrofuran, diethyl ether, dioxane, ethyl acetate, toluene, methylene chloride, dichloroethane, chloroform, N,N-dimethylacetamide, 1,3-dimethyl-2-imidazolidine, 1-methyl-2-pyrrolidinone, 1,2-dimethoxyethane, xylene,

or a combination thereof.

The reaction of a compound of formula [32] with a compound of formula [33] can be carried out in a suitable solvent in the presence of a base or a catalyst.

Such base may include inorganic bases such as an alkali metal hydride such as sodium hydride, an alkali metal carbonate such as sodium carbonate and potassium carbonate, an alkali metal amide such as sodium amide and lithium amide, an alkali metal alkoxide such as sodium methoxide, an alkali metal such as sodium, an alkali metal hydroxide such as sodium hydroxide and potassium hydroxide, or an alkyl alkali metal such as n-butyllithium, and the like. Or one can use an organic base such as triethylamine, diisopropylethylamine, morpholine, N-methylmorpholine, pyridine, dimethylaminopyridine, and the like.

Such catalyst may include palladium catalyst such as dichlorobis(triphenylphosphine)palladium, palladium acetate, palladium chloride, tetrakis(triphenylphosphine)palladium, bis(tri-t-butylphosphine)palladium, tris(dibendilideneacetone)dipalladium and the like; or copper iodide, etc. Further, for facilitating the reaction, one may add phosphorus compounds such as triphenylphosphine, 2-dicyclohexylphosphino-2′,4′,6′-triisopropylbiphenyl, 2-dicyclohexylphosphino-2′-(N,N-dimethylamino)biphenyl, and 2,2′-bis(diphenylphosphino)-1,1′-binaphthyl, or the like.

This reaction suitably proceeds at 0° C. to 200° C., particularly at room temperature to 110° C.

The solvent may be any solvent which does not have a negative impact on the reaction. Examples include acetonitrile, methanol, ethanol, isopropyl alcohol, n-propyl alcohol, acetone, N,N-dimethylformamide, dimethylsulfoxide, tetrahydrofuran, diethyl ether, dioxane, ethyl acetate, toluene, methylene chloride, dichloroethane, chloroform, N,N-dimethylacetamide, 1,3-dimethyl-2-imidazolidine, 1-methyl-2-pyrrolidinone, 1,2-dimethoxyethane, xylene, N-methylpyrrolidone or a combination thereof.

The reaction of a compound of formula [34] with a compound of formula [17] or a salt thereof can be carried out in the same manner as described above in Scheme A1 for reacting a compound of formula [16] with a compound of formula [17] or a salt thereof

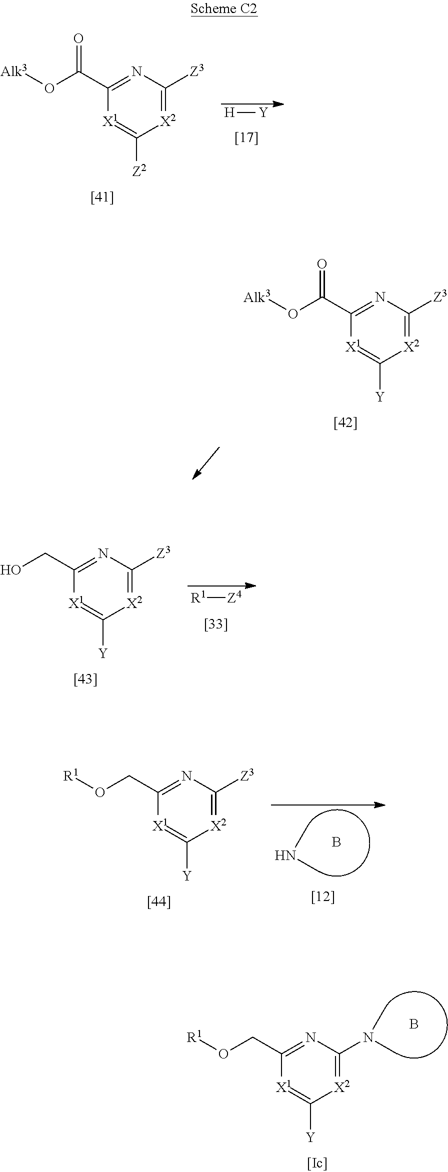

Compounds of formula [Ic] can be prepared by the following manners.

First, a compound represented by formula [41]:
wherein Alk³ is lower alkyl group, and the other symbols have the same meaning as defined above,
   is reacted with a compound represented by formula [17]:

wherein the symbols have the same meaning as defined above,

or a salt thereof to provide a compound represented by formula [42]:

wherein the symbols have the same meaning as defined above.

A compound of formula [42] is subjected to reduction reaction to provide a compound represented by formula [43]:

wherein the symbols have the same meaning as defined above.

A compound of formula [43] is reacted with a compound represented by formula [33]:

wherein the symbols have the same meaning as defined above,

to provide a compound represented by formula [44]:

wherein the symbols have the same meaning as defined above.

A compound of formula [44] is reacted with a compound represented by formula [12]:

wherein the symbols have the same meaning as defined above, to provide a compound of formula [Ic] which is optionally converted to a pharmaceutically acceptable salt.

The reactions in Scheme C2 can be carried out as described below.

The reaction of a compound of formula [41] with a compound of formula [17] or a salt thereof can be carried out in the same manner as described above in Scheme A1 for reacting a compound of formula [16] with a compound of formula [17] or a salt thereof.

The reduction reaction of a compound of formula [42] can be carried out in the presence of reducing agents (sodium borohydride, lithium borohydride, lithium aluminium hydride, diisopropyl aluminum hydride and the like) in a suitable solvent.

This reaction suitably proceeds at −78° C. to 60° C., particularly at 0° C. to room temperature.

The solvent may include hexane, diethyl ether, tetrahydrofuran, dioxane, 1,2-dimethoxyethane, methanol, ethanol, toluene, or a combination thereof.

The reaction of a compound of formula [43] with a compound of formula [33] can be carried out in the same manner as described above in Scheme C1 for reacting a compound of formula [32] with a compound of formula [33].

The reaction of a compound of formula [44] with a compound of formula [12] or a salt thereof can be carried out in the same manner as described above in Scheme A1 for reacting a compound of formula [11] with a compound of formula [12] or a salt thereof.

Raw material compounds in the above preparation schemes (Scheme A1, Scheme A2, Scheme B, Scheme C1 and Scheme C2) can be prepared by procedures known in the art and/or recited in Reference Examples described hereinafter. Also, compounds of formula [I] or [I⁰] prepared by the above preparation schemes (Scheme A1, Scheme A2, Scheme B, Scheme C1 and Scheme C2) can be allowed to structural conversion into the other compounds of formula [I] or [I⁰] by the procedures recited in Examples described hereinafter and/or known in the art, or a combination thereof.

The compounds of the present invention or raw material compounds thereof can be isolated and purified as the free form (free base or free acid) or as the salt thereof. The salt can be prepared by salt formation treatments usually employed. For instance, the salt formation treatment can be carried out by adding an acid or a base or the solution thereof to the solution or suspension of the compound of the present invention. Preferable acid is a pharmaceutically acceptable salt, which includes hydrochloric acid, sulfuric acid, nitric acid, phosphoric acid, hydrobromic acid, acetic acid, fumaric acid, oxalic acid, citric acid, methanesulfonic acid, benzenesulfonic acid, p-toluenesulfonic acid and maleic acid. Preferable base is a pharmaceutically acceptable salt, which includes alkali metal salts such as sodium salts and potassium salts; and alkaline earth metal salts such as calcium salts. A solvent of the solution or suspension of the compound of the present invention may be any solvent which does not have a negative impact on the salt formation treatment. Examples include water; alcohol such as methanol, ethanol, and propanol; ester such as ethyl acetate; ether such as diethyl ether, dioxane, and tetrahydrofuran; dichrormethane; and chloroform, or a combination thereof.

The isolation and purification can be carried out by usual chemical procedures such as extraction, concentration, crystallization, filtration, recrystallization and various chromatography.

The compounds of formula [I⁰] or [I] or a pharmaceutically acceptable salt thereof according to the present invention possess excellent PDE 10 inhibitory activity, that is, inhibitory activity on the enzyme activity of phosphodiesterase 10 (PDE10, more specifically PDE10A), in mammals. The compounds of formula [I⁰] or [I] or a pharmaceutically acceptable salt thereof according to the present invention are also highly selective for PDE10.

Also, the compounds of formula [I⁰] or [I] or a pharmaceutically acceptable salt thereof in the present invention exhibit various pharmacological efficacies through their PDE10 inhibitory activity. Accordingly, a pharmaceutical composition comprising the compounds of formula [I⁰] or [I] or a pharmaceutically acceptable salt thereof as an active ingredient can be used to inhibit PDE 10 activity. Further, said pharmaceutical composition can be used for the treatment or prophylaxis of diseases or conditions which are expected to be ameliorated by inhibition of PDE10 activity.

As a disease or condition which is expected to be ameliorated by inhibition of PDE10 activity, there may be mentioned, for example:

Psychotic disorder such as schizophrenia:
for example, schizophrenia, schizophreniform disorder, delusional disorder, substance-induced psychotic disorder, personality disorder of the paranoid type or schizoid type, etc;

Anxiety disorder:
for example, panic disorder, agoraphobia, specific phobia, social phobia, obsessive-compulsive disorder, post-traumatic stress disorder, acute stress disorder, generalized anxiety disorder, etc;

Drug addiction:
for example, addiction to alcohol, amphetamine, cocaine, or opiate, etc;

Disorders comprising deficient cognition as a symptom:
for example, dementia (including Alzheimer's disease, multi-infarct dementia, etc.), delirium, amnestic disorder, post-traumatic stress disorder, mental retardation, a learning disorder, attention deficit hyperactivity disorder (ADHD), age-related cognitive decline, etc; and

Mood disorder:
for example major depressive disorder, dysthymic disorder, minor depressive disorder, bipolar disorder (including bipolar I disorder, bipolar II disorder), cyclothymic disorder, etc; or

Mood episode:
for example, major depressive episode, manic or mixed mood episode, hypomanic mood episode, etc.

Of these diseases and conditions, one may wish to focus on treating the following diseases by using the compounds of the invention:

Schizophrenia:

Anxiety disorder:
for example, panic disorder, social phobia, obsessive-compulsive disorder, post-traumatic stress disorder, generalized anxiety disorder;

Drug addiction:

Disorders comprising deficient cognition as a symptom:
for example, dementia (including Alzheimer's disease, etc.), learning disorder, attention deficit hyperactivity disorder (ADHD) and age-related cognitive decline; and

Mood disorder:
for example, major depressive disorder, dysthymic disorder, minor depressive disorder, bipolar disorder.

Of these diseases and conditions, one may wish to focus particularly on treating the following diseases by using the compounds of the invention:

Schizophrenia:

Anxiety disorder:
for example, panic disorder, social phobia, obsessive-compulsive disorder, post-traumatic stress disorder, generalized anxiety disorder; and

Mood disorder:
for example, major depressive disorder, dysthymic disorder, minor depressive disorder, bipolar disorder.

One may wish to focus more particularly on treating schizophrenia by using the compounds of the invention.

In addition, the compounds of the invention may be used to treat a disease or condition which is expected to be ameliorated by inhibition of PDE10 activity, including for example;

movement disorder or neurodegenerative disorder

including dyskinesia associated with dopamine agonist therapy;

Huntington's disease;

Parkinson's disease; and

restless leg syndrome.

In addition, the compounds of the invention may be used to treat a disease or condition which is expected to be ameliorated by inhibition of PDE10 activity, including for example, cancer.

In addition, the compounds of the invention may be used to treat a disease or condition which is expected to be ameliorated by inhibition of PDE10 activity, including for example;
type 1 or type 2 diabetes (or non-insulin dependent diabetes (NIDDM));
impaired glucose tolerance (IGT);
impaired fasting glucose (IGF);
metabolic syndrome; and
metabolism related disorders including excess of body weight or excess of body fat in obese patient.

Also within the scope of this invention is a method for treating or preventing a disease or condition by administering to a patient (or a subject) in need thereof an effective amount of a compound of formula [I⁰] or [I] or a pharmaceutically acceptable salt thereof.

Also, use of a compound of formula [I⁰] or [I] or a pharmaceutically acceptable salt thereof for the manufacture of a medicament are also encompassed within a scope of the present invention.

Inhibitory action on PDE10 and pharmacological effects of the compounds of the present invention can be confirmed by known methods and equivalent methods thereto.

For example, measurements of PDE10 inhibitory activities can be carried out by the method described below in Experimental Example 1 or by methods disclosed in literature. See for example, Fujishige et al., Eur. J. Biochem., vol. 266, pp. 1118-1127, 1999, and Mukai et al., Br. J. Pharmacol., vol. 111, pp. 389-390, 1994.

Further, selectivity of the compounds described herein for PDE10 may be evaluated by using the methods disclosed in the literature. See for example, Kotera et al., Biochem. Pharmacol., vol. 60, pp. 1333-1341, 2000; Sasaki et al., Biochem. Biophys. Res. Commun., vol. 271, pp. 575-583, 2000; Yuasa et al., Journal of Biological Chemistry, vol. 275, pp. 31469-31479, 2000; Gamanuma et al., Cellular Signaling, vol. 15, pp. 565-574, 2003.

Pharmacological effects on the symptoms of schizophrenia can be detected by the following in vivo test systems using mouse or rat.

MK-801-induced locomotor activity:
[O'Neil and Shaw, Psychopharmacology, 1999, 145:237-250].

Apomorphine-induced locomotor activity:
[Geyer et al., Pharmacol. Biochem. Behav., 1987, 28:393-399; Ellenbroek, Pharmacol. Ther., 1993, 57:1-78].

Conditioned avoidance response:
[Moor et al., J. Pharmacol. Exp. Ther., 1992, 262:545-551].

Pharmacological effects to improve the deficient cognition in schizophrenia etc can be detected by the following in vivo test systems using mouse or rat.

MK-801-induced Isolation rearing Prepulse inhibition (PPI) deficit:
[Mansbach and Geyer, Neuropsychopharmacology, 1989, 2:299-308; Bakshi et al., J. Pharmacol. Exp. Ther., 1994, 271:787-794; Bubenikova et al., Pharmacol. Biochem. Behav., 2005, 80:591-596].

Isolation rearing-induced Prepulse inhibition (PPI) deficit:
[Cilia et al., Psychopharmacology, 2001, 156:327-337].

MK-801-induced deficit in Novel object recognition task (NOR):
[Karasawa et al., Behav. Brain. Res., 2008, 186:78-83].

The compounds of formula [I⁰] or [I] or a pharmaceutically acceptable salt thereof can be formulated into a conventional pharmaceutical preparation such as a tablet, granule, capsule, powder, solution, suspension, emulsion, inhalent, injectibles and drops, etc, by mixing the compound(s) with an inert pharmaceutically acceptable carrier suitable for each administration route.

Examples of such carriers include any conventional pharmaceutically acceptable materials, such as binders (gum Alabicum, gelatin, sorbitol, polyvinylpyrrolidone, etc.), excipients (lactose, sucrose, corn starch, sorbitol, etc.), lubricants (magnesium stearate, talc, polyethyleneglycol, etc.), disintegrators (potato starch, etc.) and the like.

In case of injectibles and drops, the compounds of the present invention can be mixed with distilled water for injection, physiological saline, aqueous glucose solution and the like.

The administration route of the compounds of formula [I⁰] or [I] or a pharmaceutically acceptable salt thereof is not limited to particular route. They can be administered orally or parenterally (for example, through intravenous, intramuscular, subcutaneous, transdermal, transnasal, transmucosal or enteral route).

Further, in case of treating a central nervous system (CNS) disease, the drug can be directly or indirectly introduced into the brain, by bypassing the blood-brain barrier (BBB). Examples of those methods include intracerebroventricular (i.c.v.) administration, and an administration method accompanying intravenous injection of hypertonic solution which enables temporary opening of the BBB (osmotic opening).

When a compound of formula [I⁰] or [I] or a pharmaceutically acceptable salt thereof is used for medical use, the dosage of the compound may be determined in accordance with the potency or property of that compound, to establish a dosage range which is effective enough for achieving the desired pharmacological efficacy. The dosage may vary depending on the administration route, age, bodyweight, and condition of the patient. A usual dosage range will be, for example, a range of 0.001 to 300 mg/kg per day.

The method of treatment or prophylaxis using a compound of the present invention is applied to a human. However, it may also be applied to mammals other than a human.

Hereinafter, the present invention is illustrated in more detail by the following Examples. The examples are given to illustrate the invention, but should not be construed to limit it. Reference is made to the claims for determining what is reserved to the inventors.

### EXAMPLES

### Experimental Example 1

### Measurement of PDE10 Inhibitory Activity

(1) The enzyme PDE10 (PDE10A) was isolated and prepared from bovine corpus striatum, according to the methods described in references Fujishige et al., Eur. J. Biochem., vol. 266, pp. 1118-1127, 1999. The enzyme solution obtained was used for a PDE assay.

The PDE assay was performed according to the method described in Kotera et al. (Kotera et al., Biochem. Pharmacol., vol. 60, pp. 1333-1341, 2000), by the radiolabeled nucleotide method.

Specifically, the measurements of the inhibitory activities were carried out in the following method.

(Method) The test compounds were dissolved in dimethyl sulfoxide (DMSO). 2 μl of the compound solution was added to 96 well plate, and the reaction mixture (20 μL of PDE enzyme solution in 50 mM Tris-HCl, pH 8.0, 40 μL of the assay buffer (50 mM Tris-HCl, pH 8.0, 2 mM MgCl2, 0.07% 2-mercaptoethanol, and 0.825 mg/mL bovine serum albumin), and 20 μL of 1 mg/mL snake venom) was added to the 96 well plate. The enzyme reaction was started by adding and mixing with substrate solution of 20 μL containing approximate 35 nM [5′,8-3H]cAMP in 50 mM Tris-HCl, pH 8.0. The final concentration of cAMP in the reaction mixtures was 7 nM. The reaction mixtures were incubated at room temperature for 90 min under dark conditions. After incubation, the reaction was stopped by adding 100 μL of methanol and resultant solutions were applied to filter plate containing Dowex (1×8 200-400) and centrifuged. 50 μL of the eluate together with wash eluate with additional 100 μL methanol was collected in another plate and the radioactivity was measured with 250 μL of scintillant.

(2) The compounds in the Examples below were tested for PDE inhibition using the Method described above.

They showed an IC₅₀ value of 2 nM or less. The IC₅₀ values of some preferred compounds are given in the following table.

| Example No | IC50 (nM) |
|---|---|
| 1.001 | 0.10 |
| 1.003 | 0.60 |
| 1.007 | 0.090 |
| 1.010 | 0.48 |
| 1.020 | 0.073 |
| 1.024 | 0.039 |
| 1.041 | 0.66 |
| 1.048 | 0.040 |
| 1.050 | 0.14 |
| 1.064 | 0.048 |
| 1.074 | 0.0033 |
| 1.078 | 0.047 |
| 1.084 | 0.011 |
| 1.090 | 0.36 |
| 1.093 | 0.30 |
| 1.094 | 0.17 |
| 1.095 | 0.79 |
| 1.099 | 0.10 |
| 1.101 | 0.46 |
| 4.003 | 0.031 |
| 5.002 | 0.61 |
| 6.001 | 0.22 |

### Example 1.001

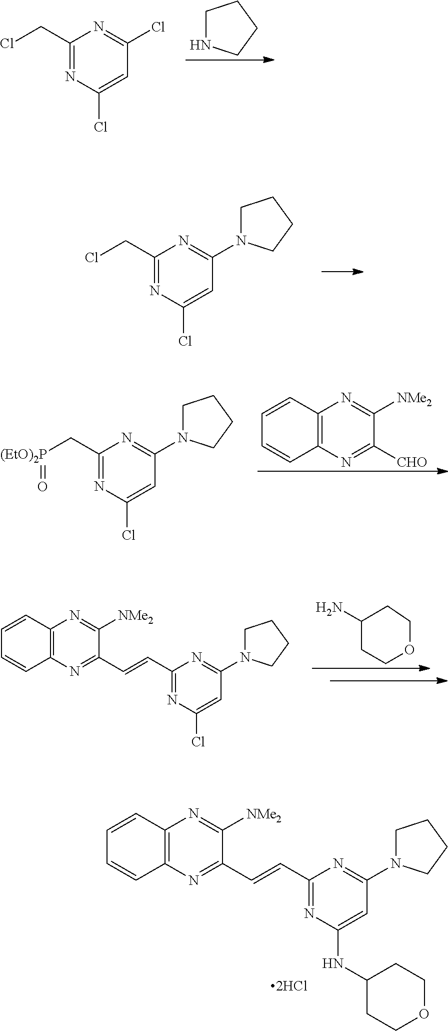

(1) To a solution of 4,6-dichloro-2-(chloromethyl)pyrimidine (see J. Chem. Soc., C 1968, 2188 and Pharm. Chem. J. 1998, 32, 621; 37 g, 0.187 mol) in N,N-dimethylformamide (550 mL) was added triethylamine (37.8 g, 0.375 mol), followed by pyrrolidine (14.0 g, 0.197 mol) at 0° C. After being stirred for 3 hour at −2° C., the reaction mixture was poured into cold water (1000 mL), and the mixture was extracted with ethyl acetate (1500 mL). The organic layer was washed with water and saturated brine, dried over magnesium sulfate, filtrated and concentrated in vacuo. The residue was purified by silica gel column chromatography (hexane:ethyl acetate=1:1) to give 4-chloro-2-(chloromethyl)-6-pyrrolidin-1-ylpyrimidine as pale yellow solid (39.0 g, 90%). MS (APCI): m/z 232/234 (M+H).

(2) To a solution of diethyl phosphite (32.5 g, 0.235 mol) in N,N-dimethylformamide (290 mL) was added sodium hydride (60% dispersion in mineral oil, 8.07 g, 0.202 mol) portionwise at 0° C., and the mixture was stirred for 40 min. Then a solution of 4-chloro-2-(chloromethyl)-6-pyrrolidin-1-ylpyrimidine (39.0 g, 0.168 mol) in N,N-dimethylformamide (200 mL) was added to the mixture and stirred for 1 hour at room temperature. The reaction mixture was poured into cold water (500 mL) and the mixture was extracted with ethyl acetate (1200 mL). The organic layer was washed with water and saturated brine, dried over magnesium sulfate, filtrated and concentrated in vacuo. The residue was purified by trituration with hexane-diethyl ether to give diethyl [(4-chloro-6-pyrrolidin-1-ylpyrimidin-2-yl)methyl]phosphonate as pale yellow solid (41.3 g, 74%). mp 68-69° C. MS (APCI): m/z 334/336 (M+H).

(3) To a solution of diethyl [(4-chloro-6-pyrrolidin-1-ylpyrimidin-2-yl)methyl]phosphonate (1.91 g, 5.72 mmol) in tetrahydrofuran (14 mL) and N,N-dimethylformamide (14 mL) was added potassium tert-butoxide (705 mg, 6.28 mmol) in one portion at 0° C. After being stirred for 30 min at 0° C., a solution of 3-dimethylaminoqunoxaline-2-carbaldehyde (1.15 g, 5.71 mmol) in tetrahydrofuran (7 mL) and N,N-dimethylformamide (7 mL) was added. The reaction mixture was stirred for 2 hour at 0° C., and then water (168 mL) was added. The resulting precipitate was collected and washed with water (100 mL), and dissolved to dichloromethane (100 mL). The organic layer was dried over magnesium sulfate, filtrated and concentrated in vacuo. The residue was purified by trituration with diethyl ether to give 3-[(E)-2-(4-chloro-6-pyrrolidin-1-ylpyrimidin-2-yl)vinyl]-N,N-dimethylquinoxalin-2-amine as yellow crystals (1.63 g, 75%). mp 196-197° C. MS (APCI): m/z 381/383 (M+H).

(4) A mixture of 3-[(E)-2-(4-chloro-6-pyrrolidin-1-ylpyrimidin-2-yl)vinyl]-N,N-dimethylquinoxalin-2-amine (150 mg, 0.394 mmol), 4-aminotetrahydro-2H-pyran (199 mg, 1.97 mmol), sodium tert-butoxide (57 mg, 0.593 mmol), tris(dibenzylideneacetone)dipalladium(0) (36 mg, 0.0393 mmol), and 2-dicyclohexylphosphino-2′,4′,6′-triisopropylbiphenyl (19 mg, 0.0393 mmol) in tert-butanol (4.0 mL) was heated for 5 hour at 80° C. After being cooled to ambient temperature, the reaction mixture was filtrated through celite with chloroform (15 mL). The filtrate was combined and concentrated in vacuo. The residue was purified by silica gel column chromatography (chloroform to chloroform:methanol=19:1) to give N,N-dimethyl-3-{(E)-2-[4-pyrrolidin-1-yl-6-(tetrahydro-2H-pyran-4-ylamino)pyrimidin-2-yl]vinyl}quinoxalin-2-amine as brown oil (191 mg, quant.).

(5) To a solution of N,N-dimethyl-3-{(E)-2-[4-pyrrolidin-1-yl-6-(tetrahydro-2H-pyran-4-ylamino)pyrimidin-2-yl]vinyl}quinoxalin-2-amine (191 mg, 0.394 mmol) in dichloromethane (0.5 mL) was added hydrogen chloride solution (4N in 1,4-dioxane, 0.5 mL). The resulting precipitate was collected and washed with diethyl ether to give N,N-dimethyl-3-{(E)-2-[4-pyrrolidin-1-yl-6-(tetrahydro-2H-pyran-4-ylamino)pyrimidin-2-yl]vinyl}quinoxalin-2-amine dihydrochloride (the compound of Example 1.001 listed in Table 1 as described hereinafter) as a yellow powder (161 mg, 79%). ¹H NMR (DMSO-d₆): δ 1.52 (2H, br), 1.91-2.01 (6H, m), 3.09 (6H, s), 3.47 (4H, t, J=10.8 Hz), 3.91 (4H, d, J=11.2 Hz), 5.62 (1H, br), 7.55-7.58 (1H, m), 7.69-7.72 (1H, m), 7.76-7.78 (1H, m), 7.92 (1H, d, J=8.3 Hz), 8.08 (1H, br), 8.21 (1H, d, J=15.4 Hz).

### Example 1.002

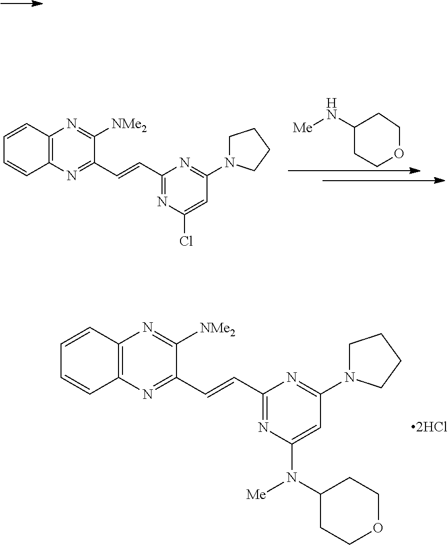

(1) The preparation was performed in the same manner as described in the above Example 1.001 (1) to (3) to give 3-[(E)-2-(4-chloro-6-pyrrolidin-1-ylpyrimidin-2-yl)vinyl]-N,N-dimethylquinoxalin-2-amine.

(2) A mixture of 3-[(E)-2-(4-chloro-6-pyrrolidin-1-ylpyrimidin-2-yl)vinyl]-N,N-dimethylquinoxalin-2-amine (150 mg, 0.394 mmol), N-methyl-4-aminotetrahydro-2H-pyran (223 mg, 1.97 mmol), sodium tert-butoxide (57 mg, 0.593 mmol), palladium(II)acetate (9 mg, 0.0593 mmol), and 2-dicyclohexylphosphino-2′-(N,N-dimethylamino)biphenyl (31 mg, 0.0788 mmol) in 1,4-dioxane (4.0 mL) was heated for 5 hour at 100° C. After being cooled to ambient temperature, the reaction mixture was filtrated through celite with chloroform (15 mL). The filtrate was combined and concentrated in vacuo. The residue was purified by silica gel column chromatography (chloroform to chloroform:methanol=19:1) to give N,N-dimethyl-3-((E)-2-{4-[methyl(tetrahydro-2H-pyran-4-yl)amino]-6-pyrrolidin-1-yl-pyrimidin-2-yl}vinyl)quinoxalin-2-amine as brown amorphous powder (111 mg, 61%).

(3) The preparation of the hydrogen chloride salt was performed in the same manner as described in Example 1.001 (5) to give N,N-dimethyl-3-((E)-2-{4-[methyl(tetrahydro-2H-pyran-4-yl)amino]-6-pyrrolidin-1-yl-pyrimidin-2-yl}vinyl)quinoxalin-2-amine dihydrochloride (the compound of Example 1.002 listed in Table 1 as described hereinafter) as a yellow powder. ¹H NMR (DMSO-d₆): δ 1.60-1.63 (2H, m), 1.86-1.94 (2H, m), 2.02 (4H, br), 3.02 (2H, br), 3.11 (6H, s), 4.01 (2H, br), 5.11 (1H, br), 5.57 (1H, br), 7.56-7.59 (1H, m), 7.69-7.72 (1H, m), 7.77-7.78 (1H, m), 7.92 (1H, d, J=8.3 Hz), 7.96 (1H, d, J=14.6 Hz), 8.22 (1H, d, J=15.1 Hz).

### Examples 1.003 to 1.047

The compounds of Examples 1.003 to 1.047 listed in Table 1 as described hereinafter were obtained in the similar manner as described in the above Example 1.001.

### Example 1.048

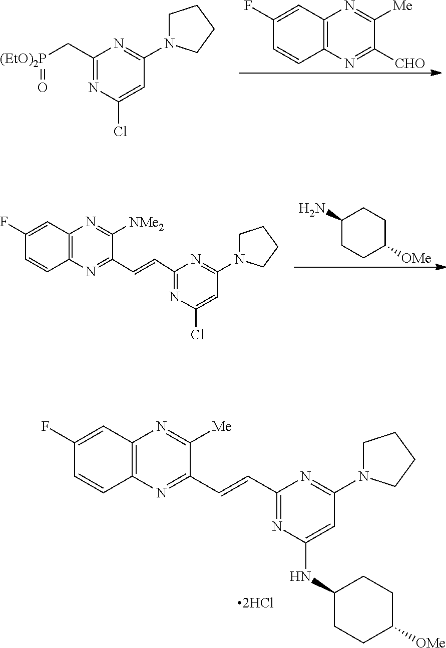

(1) To a solution of diethyl [(4-chloro-6-pyrrolidin-1-ylpyrimidin-2-yl)methyl]phosphonate (1.59 g, 4.76 mmol) in tetrahydrofuran (20 mL) and N,N-dimethylformamide (20 mL) was added potassium tert-butoxide (559 mg, 4.99 mmol) in one portion at 0° C. After being stirred for 30 min at 0° C., the mixture was cooled to −78° C., and a solution of 6-fluoro-3-methylqunoxaline-2-carbaldehyde (862 mg, 4.53 mmol) in tetrahydrofuran (3 mL) and N,N-dimethylformamide (3 mL) was added. The reaction mixture was stirred for 1 hour at −78° C., and then water was added. The resulting precipitate was collected, and dissolved to chloroform. The organic layer was washed with saturated brine and dried over sodium sulfate, filtrated and concentrated in vacuo. The residue was purified by trituration with ethyl acetate to give 2-[(E)-2-(4-chloro-6-pyrrolidin-2-yl)vinyl]-6-fluoro-3-methylquinoxaline (the compound of Reference Example 3.12 listed in Table of Reference Example as described hereinafter) as pale yellow powder (1.18 g, 70%). MS (APCI): m/z 370/372 (M+H).

(2) A mixture of 2-[(E)-2-(4-chloro-6-pyrrolidin-2-yl)vinyl]-6-fluoro-3-methylquinoxaline (300 mg, 0.811 mmol), trans-4-methoxycyclohexylamine hydrochloride (403 mg, 2.43 mmol), potassium hydroxide (182 mg, 3.24 mmol), tris(dibenzylideneacetone)dipalladium(0) (74 mg, 0.081 mmol), and 2-dicyclohexylphosphino-2′,4′,6′-triisopropylbiphenyl (39 mg, 0.082 mmol) in tert-butanol (10 mL) was heated for 12 hour at 80° C. After being cooled to ambient temperature, the reaction mixture was filtrated through celite with chloroform (15 mL). The filtrate was combined and concentrated in vacuo. The residue was purified by silica gel column chromatography (chloroform:ethyl acetate=9:1 to 3:2) followed by trituration with diisopropyl ether to give 2-[(E)-2-(6-fluoro-3-methylquinoxalin-2-yl)vinyl]-N-(trans-4-methoxycyclohexyl)-6-pyrrolidin-1-ylpyrimidin-4-amine as brown solid (87 mg).

(3) To a solution of 2-[(E)-2-(6-fluoro-3-methylquinoxalin-2-yl)vinyl]-N-(trans-4-methoxycyclohexyl)-6-pyrrolidin-1-ylpyrimidin-4-amine (87 mg) in chloroform (1.8 mL) was added hydrogen chloride solution (4N in 1,4-dioxane, 0.09 mL). The resulting precipitate was collected and washed with diisopropyl ether to give 2-[(E)-2-(6-fluoro-3-methylquinoxalin-2-yl)vinyl]-N-(trans-4-methoxycyclohexyl)-6-pyrrolidin-1-ylpyrimidin-4-amine dihydrochloride (the compound of Example 1.048 listed in Table 1 as described hereinafter) as yellow powder (91 mg, 21%). ¹H NMR (CDCl₃): δ 1.41-1.48 (2H, m), 1.54-1.61 (2H, m), 2.07-2.14 (8H, m), 3.28-3.32 (1H, m), 3.34 (3H, s), 3.36 (3H, s), 3.40-3.47 (3H, m), 3.82 (2H, br), 5.09 (1H, s), 7.68 (1H, ddd, J=9.2, 8.1, 2.9 Hz), 7.73 (1H, d, J=16.1 Hz), 8.27 (1H, dd, J=9.3, 5.5 Hz), 8.31 (1H, dd, J=8.3, 2.6 Hz), 8.55 (1H, d, J=7.4 Hz), 8.82 (1H, d, J=16.1 Hz).

### Examples 1.049 to 1.077

The compounds of Examples 1.049 to 1.077 listed in Table 1 as described hereinafter were obtained in the similar manner as described in the above Example 1.001.

### Example 1.078

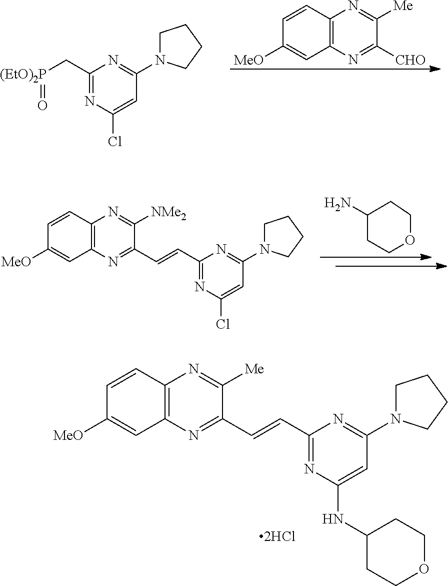

(1) To a solution of diethyl [(4-chloro-6-pyrrolidin-1-ylpyrimidin-2-yl)methyl]phosphonate (1.26 g, 3.77 mmol) in tetrahydrofuran (24 mL) and N,N-dimethylformamide (8.0 mL) was added potassium tert-butoxide (406 mg, 3.62 mmol) in one portion at 0° C. After being stirred for 15 min at 0° C., the mixture was cooled to −78° C., and a solution of 7-methoxy-3-methylqunoxaline-2-carbaldehyde (665 mg, 3.29 mmol) in tetrahydrofuran was added. The reaction mixture was stirred for 1 hour at −78° C., and then water was added. The resulting precipitate was collected, and dissolved to chloroform. The organic layer was dried over sodium sulfate, filtrated and concentrated in vacuo. The residue was purified by trituration with ethyl acetate to give 2-[(E)-2-(4-chloro-6-pyrrolidin-2-yl)vinyl]-7-methoxy-3-methylquinoxaline (the compound of Reference Example 3.20 listed in Table of Reference Example as described hereinafter) as yellow powder (973 mg, 77%).

(2) A mixture of 2-[(E)-2-(4-chloro-6-pyrrolidin-2-yl)vinyl]-7-methoxy-3-methylquinoxaline (200 mg, 0.524 mmol), 4-aminotetrahydro-2H-pyran (265 mg, 2.62 mmol), sodium tert-butoxide (76 mg, 0.79 mmol), tris(dibenzylideneacetone)dipalladium(0) (48 mg, 0.052 mmol), and 2-dicyclohexylphosphino-2′,4′,6′-triisopropylbiphenyl (25 mg, 0.052 mmol) in tert-butanol (5.0 mL) was heated for overnight at 80° C. After being cooled to ambient temperature, the reaction mixture was filtrated through celite with chloroform. The filtrate was combined and concentrated in vacuo. The residue was purified by silica gel column chromatography (hexane:ethyl acetate=1:1 to ethyl acetate) followed by trituration with diisopropyl ether to give 2-[(E)-2-(7-methoxy-3-methylquinoxalin-2-yl)vinyl]-6-pyrrolidin-1-yl-N-(tetrahydro-2H-pyran-4-yl)pyrimidin-4-amine (138 mg).

(3) To a solution of 2-[(E)-2-(7-methoxy-3-methylquinoxalin-2-yl)vinyl]-6-pyrrolidin-1-yl-N-(tetrahydro-2H-pyran-4-yl)pyrimidin-4-amine (138 mg) in chloroform (1.0 mL) was added hydrogen chloride solution (4N in 1,4-dioxane, 1.0 mL). The resulting precipitate was collected and washed with diethyl ether to give 2[(E)-2-(7-methoxy-3-methylquinoxalin-2-yl)vinyl]-6-pyrrolidin-1-yl-N-(tetrahydro-2H-pyran-4-yl)pyrimidin-4-amine dihydrochloride (the compound of Example 1.078 listed in Table 1 as described hereinafter) as yellow powder (164 mg, 60%). ¹H NMR (DMSO-d₆): δ 1.42-1.57 (2H, m), 1.88-2.08 (4H, m), 2.86 (3H, s), 3.39-3.54 (4H, m), 3.84-3.95 (2H, m), 3.96 (3H, s), 5.60 (1H, s), 7.41 (1H, s), 7.49 (1H, dd, J=2.7, 9.1 Hz), 7.91 (1H, d, J=9.4 Hz), 8.24-8.82 (2H, m).

### Examples 1.079 to 1.093

The compounds of Examples 1.079 to 1.093 listed in Table 1 as described hereinafter were obtained in the similar manner as described in the above Example 1.001.

### Example 1.094

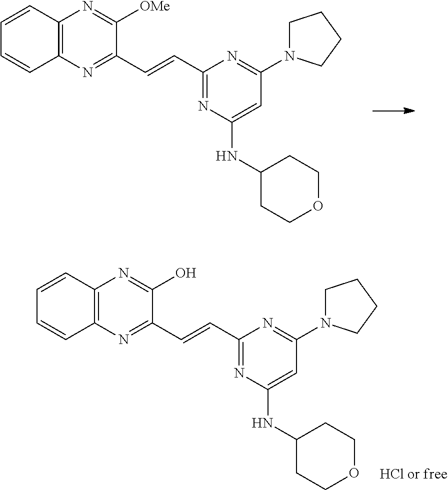

To a solution of 2-[(E)-2-(3-methoxyquinoxalin-2-yl)vinyl]-6-pyrrolidin-1-yl-N-(tetrahydro-2H-pyran-4-yl)pyrimidin-4-amine (426 mg, 0.985 mmol) in dichloromethane (1.0 mL) was added hydrogen chloride solution (4N in 1,4-dioxane, 1.0 mL). The resulting precipitate was poured into saturated sodium bicarbonate, and extracted with chloroform. The organic layer was washed with water and saturated brine, dried over sodium sulfate, filtrated and concentrated in vacuo. The residue was purified by silica gel column chromatography (chloroform to chloroform:methanol) to give 3-{(E)-2-[4-pyrrolidin-1-yl-6-(tetrahydro-2H-pyran-4-ylamino)pyrimidin-2-yl]vinyl}quinoxalin-2-ol (the free form of the compound of Example 1.095 listed in Table as described hereinafter) as a yellow powder (86 mg, 21%) and recovered starting material (137 mg, 32%).

The preparation of the hydrogen chloride salt was performed in the same manner as described in Example 1.001 (5) to give 3-{(E)-2-[4-pyrrolidin-1-yl-6-(tetrahydro-2H-pyran-4-ylamino)pyrimidin-2-yl]vinyl}quinoxalin-2-ol hydrochloride (the hydrochloride salt of the compound of Example 1.095 listed in Table 1 as described hereinafter) as a yellow powder. ¹H NMR (DMSO-d₆): δ 1.45-1.59 (2H, m), 1.83-1.94 (2H, m), 1.94-2.06 (2H, m), 3.86-3.95 (2H, m), 5.60 (1H, s), 7.34-7.42 (2H, m), 7.61 (1H, dd, J=8.2, 8.2 Hz), 7.83 (1H, d, J=8.2 Hz), 8.09-8.28 (2H, m).

### Examples 1.095 to 1.109

The compounds of Examples 1.095 to 1.109 listed in Table 1 as described hereinafter were obtained in the similar manner as described in the above Example 1.002.

### Example 2.001

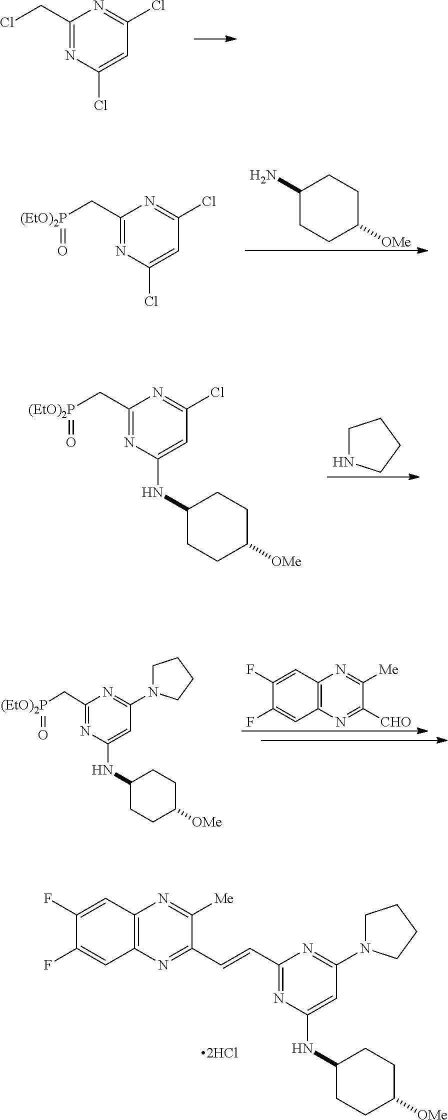

(1) A solution of 4,6-dichloro-2-(chloromethyl)pyrimidine (1.27 g, 6.44 mmol) and triethylphosphite (3.3 mL, 19.3 mmol) was heated for 17 hour at 100° C. After being cooled to ambient temperature, the reaction mixture was concentrated in vacuo. The residue was purified by silica gel column chromatography (hexane:ethyl acetate=1:1 to 1:2) to give diethyl [(4,6-dichloropyrimidin-2-yl)methyl]phosphonate as colorless oil (1.31 g, 68%). MS (APCI): m/z 299/301/303 (M+H).

(2) To a solution of methyl diethyl [(4,6-dichloropyrimidin-2-yl)methyl]phosphonate (397 mg, 1.33 mmol) and triethylamine (538 mg, 5.32 mmol) in N,N-dimethylformamide (4.0 mL) was added trans-4-methoxycyclohexylamine hydrochloride (330 mg, 2.0 mmol) at 0° C. After being stirred for 24 hour at room temperature, the reaction mixture was concentrated in vacuo. The residue was purified by silica gel column chromatography (chloroform:methanol=50:1) to give diethyl {[4-chloro-6-(trans-4-methoxycyclohexylamino)pyrimidin-2-yl]methyl}phosphonate as colorless solid (473 mg, 91%). MS (APCI): m/z 392/394 (M+H).

(3) A solution of diethyl {[4-chloro-4-(trans-6-methoxycyclohexylamino)pyrimidin-2-yl]methyl}phosphonate (470 mg, 1.2 mmol) and pyrrolidine (854 mg, 12.0 mmol) was heated for 18 hour at 100° C. After being cooled to ambient temperature, the reaction mixture was concentrated in vacuo. The residue was purified by silica gel column chromatography (chloroform:methanol=50:1 to 19:1) to give diethyl {[4-(trans-4-methoxycyclohexylamino)-6-pyrrolidin-1-ylpyrimidin-2-yl]methyl}phosphonate as brown oil (298 mg, 58%). MS (APCI): m/z 427 (M+H).

(4) To a solution of diethyl {[4-(trans-4-methoxycyclohexylamino)-6-pyrrolidin-1-yl-pyrimidin-2-yl]methyl}phosphonate (295 mg, 0.69 mmol) in tetrahydrofuran (5.0 mL) and N,N-dimethylformamide (5.0 mL) was added potassium tert-butoxide (163 mg, 1.45 mmol) at 0° C. After being stirred for 15 min, the mixture was cooled to −78° C., and then a solution of 6,7-difluoro-3-methylquinoxaline-2-carbaldehyde (144 mg, 0.690 mmol) was added. After being stirred for 1.5 hours at −78° C., the reaction mixture was poured into water, and the mixture was extracted with ethyl acetate. The organic layer was washed with water and saturated brine, dried over magnesium sulfate, filtrated and concentrated in vacuo. The residue was purified by silica gel column chromatography (chloroform:acetone=19:1 to 9:1) to give title compound as a yellow solid (111 mg, 34%).

The preparation of the hydrogen chloride salt was performed in the same manner as described in Example 1.001 (5) to give 2-[(E)-2-(6,7-difluoro-3-methylquinoxalin-2-yl)vinyl]-N-(trans-4-methoxycyclohexyl)-6-pyrrolidin-1-ylpyrimidin-4-amine dihydrochloride (the compound of Example 2.001 listed in Table 2 as described hereinafter) as an orange powder. ¹H NMR (DMSO-d₆): δ 1.28-1.42 (4H, br), 1.85-2.10 (8H, br), 2.89 (3H, s), 3.21 (1H, br), 3.26 (3H, s), 3.45 (1H, br), 3.60-4.30 (4H, br), 5.59 (1H, brs), 7.45-7.80 (1H, br), 8.00-8.60 (5H, m).

The compounds of Examples 1.001 to 1.109 listed in Table 1 as described hereinafter may also be obtained in the similar manner as described in the above Example 2.001. These compounds or the free form thereof may be applied to salt formulation treatment to obtain other salt forms, that is, phosphate, hydrobromate, fumarate, citrate, methanesulfonate, benzenesulfonate, p-toluenesulfonate and maleate. The example of such alternative method is as follows.

Alternative method for the preparation of the compound of Example 1.050

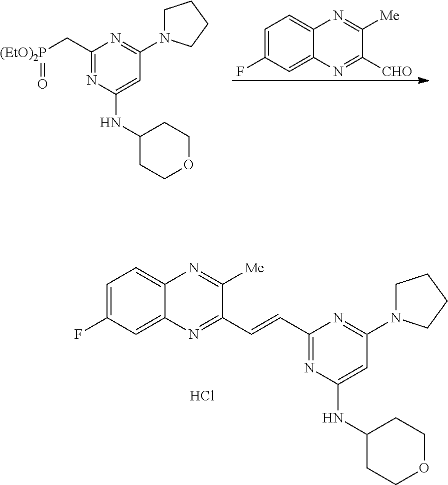

To a solution of diethyl {[4-pyrrolidin-1-yl-6-(tetrahydro-2H-pyran-4-ylamino)pyrimidin-2-yl]methyl}phosphonate (2.57 g, 6.37 mmol) in toluene (65 mL) was added lithiumtert-butoxide (540 mg, 6.69 mmol) at 0° C. After 30 min, 7-fluoro-3-methylqunoxaline-2-carbaldehyde (1.21 g, 6.37 mmol) was added, and the reaction mixture was refluxed for 2 h. After being cooled to an ambient temperature, the reaction mixture was poured into water (70 mL). The mixture was extracted with chloroform (70 mL×3), and the organic layer was washed with saturated brine (50 mL), dried over magnesium sulfate, filtrated and concentrated in vacuo. The crude was dissolved in ethanol (30 mL) and 2N aqueous hydrochloric acid (3.0 mL), and refluxed for 20 h. After cooling to an ambient temperature, the resulting precipitate was collected and washed with ethanol (30 mL) to give 2-[(E)-2-(7-fluoro-3-methylquinoxalin-2-yl)vinyl]-6-pyrrolidin-1-yl-N-(tetrahydro-2H-pyran-4-yl)pyrimidin-4-amine hydrochloride (the compound of Example 1.050 listed in Table 1 as described hereinafter) as a yellow powder (1.82 g; 61%). ¹H NMR (CDCl₃): δ 1.78-1.87 (2H, m), 1.98-2.08 (4H, m), 2.12-2.17 (2H, m), 3.07 (3H, s), 3.41 (2H, t, J=6.7 Hz), 3.55-3.61 (2H, m), 3.69-3.76 (1H, m), 3.82 (2H, t, J=6.7 Hz), 4.03-4.09 (2H, m), 5.07 (1H, s), 7.49-7.54 (1H, m), 7.68 (1H, d, J=15.7 Hz), 7.69 (1H, dd, J=9.1, 2.7 Hz), 8.00 (1H, dd, J=9.4, 5.7 Hz), 8.79 (1H, d, J=16.0 Hz), 8.87 (1H, br).

The free form of the compound above is applied to salt formulation treatment to obtain other salt forms, that is, phosphate, hydrobromate, fumarate, citrate, methanesulfonate, benzenesulfonate, p-toluenesulfonate or maleate.

### Example 3.001

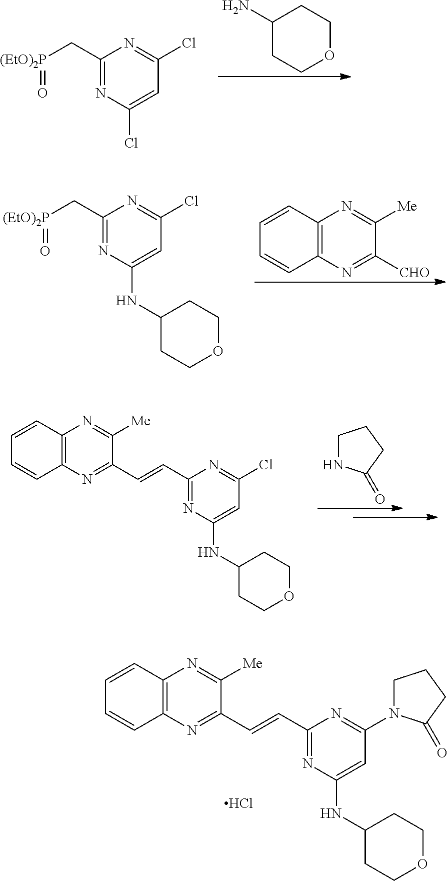

(1) The preparation was performed in the same manner as described in Example 2.001 (2) using diethyl [(4,6-dichloropyrimidin-2-yl)methyl]phosphonate (299 mg, 1.00 mmol) to give diethyl {[4-chloro-6-(tetrahydro-2H-pyran-4-ylamino)pyrimidin-2-yl]methyl}phosphonate as pale yellow solid (212 mg, 58%). MS (APCI): m/z 364/366 (M+H).

(2) The preparation was performed in the same manner as described in Example 1.001 (3) using diethyl {[4-chloro-6-(tetrahydro-2H-pyran-4-ylamino)pyrimidin-2-yl]methyl}phosphonate (208 mg, 0.570 mmol) and ethyl 3-methylquinoxaline-2-carbaldehyde (98 mg, 0.570 mmol) to give 2-[(E)-6-chloro-2-(3-methylquinoxalin-2-yl)vinyl]-N-(tetrahydro-2H-pyran-4-yl)pyrimidin-4-amine as pale yellow powder (221 mg, quant.). MS (APCI): m/z 382/384 (M+H).

(3) A mixture of 2-[(E)-6-chloro-2-(3-methylquinoxalin-2-yl)vinyl]-N-(tetrahydro-2H-pyran-4-yl)pyrimidin-4-amine (218 mg, 0.57 mmol), 2-pyrrolidinone (58 mg, 0.682 mmol), tris(dibenzylideneacetone)dipalladium(0) (52 mg, 0.0568 mmol), 4,5-bis(diphenylphosphino)-9,9-dimethylxanthene (99 mg, 0.171 mmol), and cesium carbonate (260 mg, 0.798 mmol) in 1,4-dioxane was heated for 17 hour at 100° C. After being cooled to ambient temperature, the reaction mixture was filtrated through celite with ethyl acetate. The filtrate was combined and concentrated in vacuo. The residue was purified by silica gel column chromatography (chloroform:methanol=19:1 to 5:1). The resulting crude material, 2-pyrrolidinone (73 mg, 0.858 mmol), palladium(II)acetate (13 mg, 0.0580 mmol), 2-dicyclohexylphosphino-2′,4′,6′-triisopropylbiphenyl (54 mg, 0.113 mmol), phenylboronic acid (14 mg, 0.115 mmol), and potassium carbonate (118 mg, 0.853 mmol) in tert-butanol (6.0 mL) was heated for 20 hour at 80° C. After being cooled to ambient temperature, the reaction mixture was filtrated through celite with ethyl acetate. The filtrate was combined and concentrated in vacuo. The residue was purified by silica gel column chromatography (chloroform:methanol=19:1 to 4:1) to give 1-[2-[(E)-2-(3-methylquinoxalin-2-yl)vinyl]-6-(tetrahydro-2H-pyran-4-ylamino)pyrimidin-4-yl]pyrrolidin-2-one as a pale yellow solid (113 mg, 46%).

The preparation of the hydrogen chloride salt was performed in the same manner as described in Example 1.001 (5) to give 1-[2-[(E)-2-(3-methylquinoxalin-2-yl)vinyl]-6-(tetrahydro-2H-pyran-4-ylamino)pyrimidin-4-yl]pyrrolidin-2-one hydrochloride (the compound of Example 3.001 listed in Table 2 as described hereinafter) as a yellow powder. ¹H NMR (DMSO-d₆): δ 1.45-1.57 (2H, m), 1.89-1.97 (2H, br), 2.06 (2H, m), 2.60 (2H, t, J=8.0 Hz), 2.86 (3H, s), 3.46 (2H, dt, J=1.9 Hz, 11.6 Hz), 3.91 (2H, td, J=8.1, 11.2 Hz), 4.07 (2H, t, J=7.2 Hz), 4.10-4.30 (1H, br), 7.41 (1H, s), 7.70 (1H, d, J=15.1 Hz), 7.81 (2H, m), 8.00 (1H, m), 8.09 (1H, m), 8.20 (1H, d, J=15.1 Hz).

### Example 4.001

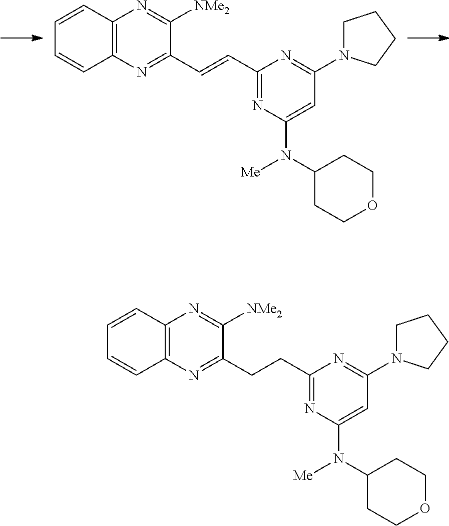

(1) A suspension of N,N-dimethyl-3-((E)-2-{4-[methyl(tetrahydro-2H-pyran-4-yl)amino]-6-pyrrolidin-1-ylpyrimidin-2-yl}vinyl)quinoxalin-2-amine dihydrochloride (98 mg, 0.184 mmol) in chloroform was basifed by adding saturated sodium bicarbonate. The organic layer was separated and concentrated in vacuo to give N,N-dimethyl-3-((E)-2-{4-[methyl(tetrahydro-2H-pyran-4-yl)amino]-6-pyrrolidin-1-ylpyrimidin-2-yl}vinyl)quinoxalin-2-amine.

(2) N,N-dimethyl-3-((E)-2-{4-[methyl(tetrahydro-2H-pyran-4-yl)amino]-6-pyrrolidin-1-ylpyrimidin-2-yl}vinyl)quinoxalin-2-amine and palladium on carbon (5%, 10 mg) in methanol was stirred for 2 hour at room temperature under a hydrogen atmosphere. The reaction mixture was filtrated and concentrated in vacuo. The residue was purified by silica gel column chromatography (hexane to hexane:ethyl acetate=19:1), followed by trituration with diethyl ether to give N,N-dimethyl-3-(2-{4-[methyl(tetrahydro-2H-pyran-4-yl)amino]-6-pyrrolidin-1-ylpyrimidin-2-yl}ethyl)quinoxalin-2-amine (the compound of Example 4.001 listed in Table 2 as described hereinafter) as a pale brown powder (35 mg, 41%). ¹H NMR (DMSO-d₆): δ 1.37 (2H, d, J=12.0 Hz), 1.69 (2H, qd, J=12.3, 4.4 Hz), 1.85 (4H, br), 2.73 (3H, s), 3.03 (6H, s), 3.09 (2H, t, J=7.5 Hz), 3.28 (4H, br), 3.88 (2H, dd, J=11.0, 3.9 Hz), 4.62-4.67 (1H, m), 5.14 (1H, s), 7.44-7.47 (1H, m), 7.55-7.58 (1H, m), 7.70 (1H, d, J=7.4 Hz), 7.80 (1H, dd, J=8.0, 0.7 Hz).

### Examples 4.002 to 4.003

The compounds of Examples 4.002 to 4.003 listed in Table 2 as described hereinafter were obtained in the same manner as described in the above Example 4.001 (2).

### Example 5.001

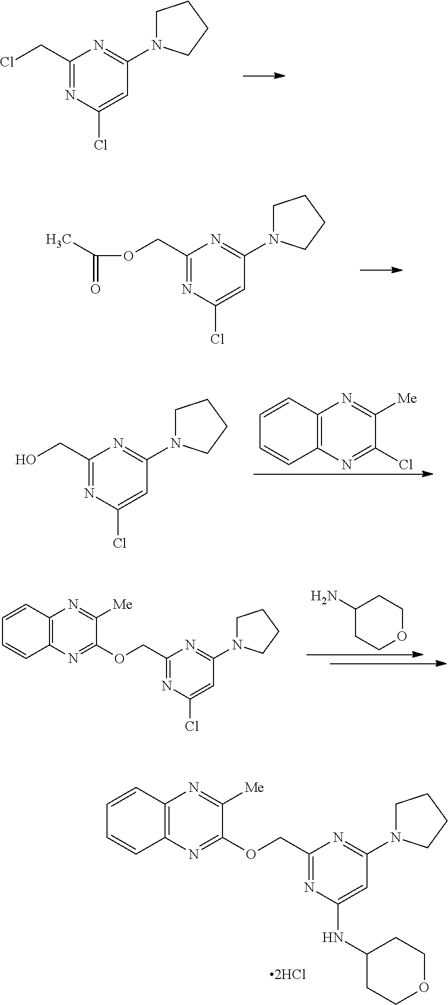

(1) A mixture of 4-chloro-2-(chloromethyl)-6-pyrrolidin-1-ylpyrimidine (2.70 g, 11.7 mmol) and potassium acetate (2.30 g, 23.4 mmol), and sodium iodide (1.93 g, 12.9 mmol) in N,N-dimethylformamide (20 mL) was stirred for 17.5 hours at room temperature. The reaction mixture was poured into water and the mixture was extracted with ethyl acetate (150 mL). The organic layer was washed with water (100 mL×2), dried over magnesium sulfate, filtrated and concentrated in vacuo to give 4-chloro-2-(acetoxymethyl)-6-pyrrolidin-1-ylpyrimidine as colorless needles (2.94 g, 98%). mp 101-103° C. MS (APCI): m/z 256/258 (M+H).

(2) To a solution of 4-chloro-2-(acetoxymethyl)-6-pyrrolidin-1-ylpyrimidine (2.94 g, 11.5 mmol) in tetrahydrofuran (50 mL) and methanol (30 mL) was added aqueous sodium hydroxide (1N, 11.7 mL, 11.7 mmol) at 0° C. The reaction mixture was stirred for 30 min at 0° C., and then poured into water. The mixture was extracted with ethyl acetate and washed with water. The organic layer was dried over magnesium sulfate, filtrated and concentrated in vacuo. The residue was purified by silica gel column chromatography (hexane:ethyl acetate=4:1 to 2:1) to give 4-chloro-2-(hydroxymethyl)-6-pyrrolidin-1-ylpyrimidine as colorless crystals (2.43 g, 99%). mp 90-93° C. MS (APCI): m/z 214/216 (M+H).

(3) To a solution of 4-chloro-2-(hydroxymethyl)-6-pyrrolidin-1-ylpyrimidine (1.00 g, 4.68 mmol) and 2-chloro-3-methylquinoxaline (1.25 g, 7.02 mmol) in N,N-dimethylformamide (10 mL) and tetrahydrofuran (20 mL) was added sodium hydride (60% dispersion in mineral oil, 281 mg, 7.02 mmol) at 0° C. The reaction mixture was stirred for 2 hour at room temperature, and then poured into cold water. The mixture was extracted with ethyl acetate and the organic layer was washed with water. The organic layer was dried over magnesium sulfate, filtrated and concentrated in vacuo. The residue was purified by silica gel column chromatography (hexane:ethyl acetate=9:1 to 7:3) to give 4-chloro-2-{[(3-methylquinoxalin-2-yl)oxy]methyl}-6-pyrrolidin-1-ylpyrimidine as red powder (1.67 g, quant.). mp 136-140° C. MS (APCI): m/z 356/358 (M+H).

(4) The preparation was performed in the same manner as described in Example 1.001 (4) using 4-chloro-2-{[(3-methylquinoxalin-2-yl)oxy]methyl}-6-pyrrolidin-1-ylpyrimidine (356 mg, 1.00 mmol) to give 2-{[(3-methylquinoxalin-2-yl)oxy]methyl}-6-pyrrolidin-1-yl-N-(tetrahydro-2H-pyran-4-yl)pyrimidin-4-amine as a pale yellow powder (335 mg, 80%).

The preparation of the hydrogen chloride salt was performed in the same manner as described in Example 1.001 (5) to give 2-{[(3-methylquinoxalin-2-yl)oxy]methyl}-6-pyrrolidin-1-yl-N-(tetrahydro-2H-pyran-4-yl)pyrimidin-4-amine dihydrochrolide (the compound of Example 5.001 listed in Table 2 as described hereinafter) as a yellow powder. ¹H NMR (DMSO-d₆): δ 1.20-1.60 (2H, br), 1.70-2.10 (6H, br), 2.71 (3H, s), 3.30-4.00 (9H, br), 5.55 (3H, brs), 7.63 (1H, t, J=7.5 Hz), 7.68 (1H, t, J=7.1 Hz), 7.74 (1H, d, J=7.7 Hz), 7.96 (1H, d, J=8.0 Hz), 8.00-8.50 (1H, br).

### Example 5.002

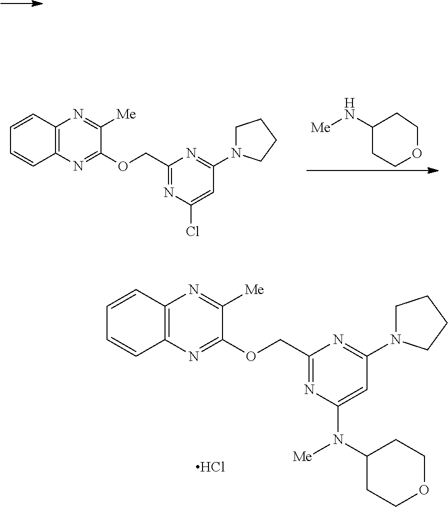

(1) The preparation was performed in the same manner as described in Example 5.001 (1) to (3) to give 4-chloro-2-{[(3-methylquinoxalin-2-yl)oxy]methyl}-6-pyrrolidin-1-ylpyrimidine.

(2) The preparation was performed in the same manner as described in Example 1.002 (2) using 4-chloro-2-{[(3-methylquinoxalin-2-yl)oxy]methyl}-6-pyrrolidin-1-ylpyrimidine (356 mg, 1.00 mmol) to give N-methyl-2-{[(3-methylquinoxalin-2-yl)oxy]methyl}-6-pyrrolidin-1-yl-N-(tetrahydro-2H-pyran-4-yl)pyrimidin-4-amine (233 mg, 54%).

The preparation of the hydrogen chloride salt was performed in the same manner as described in Example 1.001 (5) to give N-methyl-2-{[(3-methylquinoxalin-2-yl)oxy]methyl}-6-pyrrolidin-1-yl-N-(tetrahydro-2H-pyran-4-yl)pyrimidin-4-amine hydrochloride (the compound of Example 5.002 listed in Table 2 as described hereinafter) as a yellow powder. ¹H NMR (DMSO-d₆): δ 0.85-1.30 (2H, br), 1.50-1.70 (2H, br), 1.85-2.10 (4H, br), 2.70 (3H, s), 2.78 (3H, brs), 2.80-3.20 (4H, br), 3.35-3.55 (2H, br), 3.60-3.80 (2H, br), 4.38 (1H, br), 5.36 (1H, br), 5.59 (2H, brs), 7.60 (1H, t, J=7.2 Hz), 7.65 (1H, t, J=7.5 Hz), 7.70 (1H, d, J=7.9 Hz), 7.95 (1H, d, J=7.7 Hz), 10.6-14.0 (1H, br).

### Example 6.001

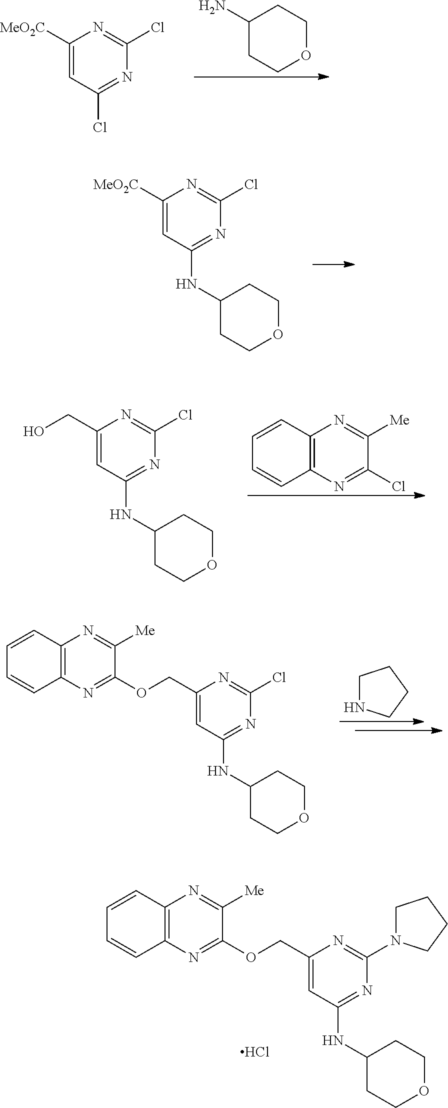

(1) To a solution of methyl 2,4-dichloropyrimidine-6-carboxylate (1.00 g, 4.83 mmol) and triethylamine (0.940 mL, 6.76 mmol) in N,N-dimethylformamide (6.0 mL) was added 4-aminotetrahydro-2H-pyran (537 mg, 5.31 mmol) at 0° C. After being stirred for 3.5 hour at 0° C., the reaction mixture was concentrated in vacuo. The residue was purified by silica gel column chromatography (hexane:ethyl acetate=1:1 to 1:2) to give methyl 2-chloro-6-(tetrahydro-2H-pyran-4-ylamino)pyrimidin-4-carboxylate as a colorless solid (1.12 g, 85%). mp 190-192° C. MS (APCI): m/z 272/274 (M+H).

(2) To a solution of methyl 2-chloro-6-(tetrahydro-2H-pyran-4-ylamino)pyrimidin-4-carboxylate (1.11 g, 4.10 mmol) in ethanol (10 mL) was added sodium borohydride (465 mg, 12.2 mmol) at 0° C. After being stirred for 2.5 hour at room temperature, the reaction mixture was poured into water. The mixture was extracted with ethyl acetate. The organic layer was dried over sodium sulfate, filtrated and concentrated in vacuo to give [2-chloro-6-(tetrahydro-2H-pyran-4-ylamino)pyrimidin-4-yl]methanol as colorless powder (1.02 g, quant.). MS (APCI): m/z 244/246 (M+H).

(3) The preparation was performed in the same manner as described in Example 5.001 (3) using [2-chloro-6-(tetrahydro-2H-pyran-4-ylamino)pyrimidin-4-yl]methanol (487 mg, 2.00 mmol) and 2-chloro-3-methylquinoxaline (536 mmol, 3.00 mmol) to give 6-[(3-methylquinoxalin-2-yl)oxy]methyl-N-(tetrahydro-2H-pyran-4-yl)pyrimidin-4-amine as pale brown powder (790 mg, quant.). MS (APCI): m/z 386/388 (M+H).

(4) The preparation was performed in the same manner as described in Example 2 using 6-[(2-chloro-3-methylquinoxalin-2-yl)oxy]methyl-N-(tetrahydro-2H-pyran-4-yl)pyrimidin-4-amine (386 mg, 1.00 mmol) and pyrrolidine (213 mg, 3.00 mmol) to give 6-[(3-methylquinoxalin-2-yl)oxy]methyl-2-pyrrolidin-1-yl-N-(tetrahydro-2H-pyran-4-yl)pyrimidin-4-amine as a pale yellow powder (308 mg, 73%).

The preparation of the hydrogen chloride salt was performed in the same manner as described in Example 1.001 (5) to give 6-[(3-methylquinoxalin-2-yl)oxy]methyl-2-pyrrolidin-1-yl-N-(tetrahydro-2H-pyran-4-yl)pyrimidin-4-amine hydrochloride (the compound of Example 6.001 listed in Table 3 as described hereinafter) as a yellow powder. ¹H NMR (DMSO-d₆): δ 1.43-1.58 (2H, m), 1.84-2.15 (6H, m), 2.69 (3H, s), 3.41 (2H, m), 3.55-3.70 (4H, m), 3.84-3.92 (2H, m), 4.09 (1H, m), 5.51 (2H, s), 6.35 (1H, s), 7.66 (1H, m), 7.72 (1H, m), 7.82 (1H, m), 7.98 (1H, d, J=8.2 Hz), 8.95 (1H, d, J=7.0 Hz), 11.82 (1H, br).

### Reference Example 1.01

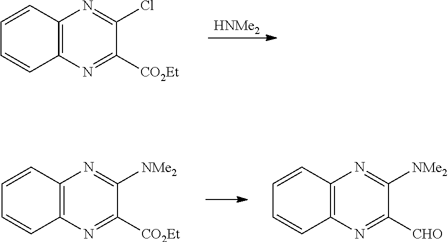

(1) To a solution of ethyl 3-chloroquinoxaline-2-carboxylate (see J. Chem. Soc. 1945, 622; 12.3 g, 52.0 mmol) and triethylamine (8.70 mL, 62.4 mmol) in N,N-dimethylformamide (52 mL) was added aqueous dimethylamine (50%, 6.60 mL, 62.7 mmol) at room temperature. After being stirred for 3 hour at room temperature, the reaction mixture was poured into water (500 mL), and the mixture was extracted with ethyl acetate (2000 mL). The organic layer was washed with water, dried over sodium sulfate, filtrated and concentrated in vacuo. The residue was purified by silica gel column chromatography (hexane:ethyl acetate=4:1) to give ethyl 3-(dimethylamino)quinoxaline-2-carboxylate as a pale yellow oil (12.6 g, 99%). MS (APCI): m/z 246 (M+H).

(2) To a solution of ethyl 3-(dimethylamino)quinoxaline-2-carboxylate (6.32 g, 25.8 mmol) in tetrahydrofuran (80 mL) was added diisobutylaluminium hydride (1.01 M solution in toluene, 77.0 mL, 77.8 mmol) dropwise over 10 min at −78° C. The reaction mixture was stirred for 1 hour at −78° C., and then methanol (77 mL) was added and allowed to warm to room temperature. The precipitate was removed through celite with ethyl acetate (1000 mL) and diethyl ether (1000 mL). The filtrate was combined and concentrated in vacuo. The residue was purified by silica gel column chromatography (hexane:ethyl acetate=9:1 to 1:1) to give 3-dimethylaminoqunoxaline-2-carbaldehyde (the compound of Reference Example 1.01 listed in the Table of Reference Examples as described hereinafter) as a yellow solid (4.85 g, 94%).

### Reference Examples 1.02 to 1.03

The compounds of Reference Examples 1.02 to 1.03 listed in the Table of Reference Examples as described hereinafter were obtained in the same manner as described in the above Reference Example 1.01.

### Reference Example 1.04

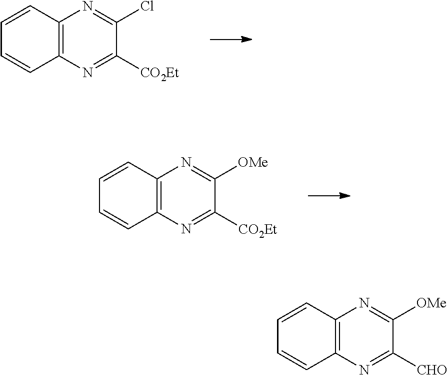

(1) To a solution of ethyl 3-chloroquinoxaline-2-carboxylate (2.00 g, 8.41 mmol) was added sodium methoxide (28% in methanol, 3.60 g, 18.7 mmol) at 0° C. After being stirred for 1 hour at room temperature, the reaction mixture was diluted with dichloromethane (200 mL). The solution was neutralized with ammonium chloride and filtrated through celite. The filtrate was combined and concentrated in vacuo. The residue was purified by silica gel column chromatography (hexane:ethyl acetate=9:1 to 3:2), followed by trituration with hexane to give ethyl 3-methoxyquinoxaline-2-carboxylate as colorless powder (1.37 g, 74%). MS (APCI): m/z 219 (M+H).

(2) The preparation was performed in the same manner as described in Reference Example 1.01 (2) using ethyl 3-methoxyquinoxaline-2-carboxylate (200 mg, 0.917 mmol) to give ethyl 3-methoxyquinoxaline-2-carbaldehyde (the compound of Reference Example 1.04 listed in the Table of Reference Examples as described hereinafter) as a colorless powder (102 mg, 59%).

### Reference Example 1.05

The compound of Reference Example 1.05 listed in the Table of Reference Examples as described hereinafter was obtained in the same manner as described in the above Reference Example 1.04.

### Reference Example 1.06

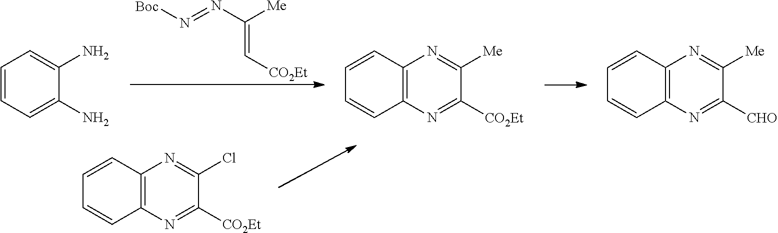

(1A) Method A: This preparation was performed in the same manner as described in Helv. Chim. Acta. 2001, 84, 2379 to give ethyl 3-methylquinoxaline-2-carboxylate.

(1B) Method B: A suspension of ethyl 3-chloroquinoxaline-2-carboxylate (11.5 g, 48.6 mmol), trimethylboroxine (6.06 g, 48.6 mmol), [1,1′-bis(diphenylphosphino)ferrocene]dichloropalladium(II) (1.98 g, 2.42 mmol), and potassium carbonate (13.4 g, 97.0 mmol) in 1,4-dioxane (162 mL) was heated for 4.5 hour at 115° C. After being cooled to ambient temperature, the reaction mixture was filtrated through celite with ethyl acetate (500 mL). The filtrate was combined and concentrated in vacuo. The residue was purified by silica gel column chromatography (hexane:ethyl acetate=9:1 to 2:1) followed by recrystallization from ethanol-water (1/4) to give ethyl 3-methylquinoxaline-2-carboxylate as colorless crystals (8.36 g, 80%). mp 74-75° C. MS (APCI): m/z 217 (M+H).

(2) The preparation was performed in the same manner as described in Reference Example 1.01 (2) using ethyl 3-methylquinoxaline-2-carboxylate (1.67 g, 7.71 mmol) to give 3-methylquinoxaline-2-carbaldehyde (the compound of Reference Example 1.06 listed in the Table of Reference Examples as described hereinafter) as pale yellow needles (680 mg, 51%).

### Reference Example 1.07

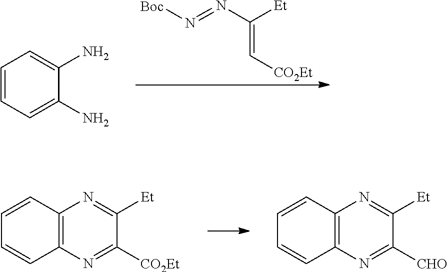

(1) The preparation was performed in the same manner as described in Helv. Chim. Acta. 2001, 84, 2379, and was carried out as follows. To a solution of tert-butyl (E)-[(1E)-1-ethyl-3-ethoxy-3-oxoprop-1-en-1-yl]diazenecarboxylate (see Synlett. 2003, 8, 1183; 1.50 g, 6.19 mmol) in tetrahydrofuran (30 mL) was added 1,2-phenylenediamine (683 mg, 6.19 mmol) at room temperature. After being stirred for 22 hour, the reaction mixture was poured into water and extracted with ethyl acetate. The organic layer was combined and dried over magnesium sulfate, filtrated and concentrated in vacuo. The residue was purified by silica gel column chromatography (hexane to hexane:ethyl acetate=6:1) to give ethyl 3-ethylquinoxaline-2-carboxylate as pale yellow solid (923 mg, 69%). mp 53-54° C. MS (APCI): m/z 217 (M+H).

(2) The preparation was performed in the same manner as described in Reference Example 1.01 (2) using ethyl 3-ethylquinoxaline-2-carboxylate (2.08 g, 9.62 mmol) to give 3-ethylquinoxaline-2-carbaldehyde (the compound of Reference Example 1.07 listed in the Table of Reference Examples as described hereinafter) as a yellow solid (908 mg, 51%).

### Reference Example 1.08

The compound of Reference Example 1.08 listed in the Table of Reference Examples as described hereinafter was obtained in the same manner as described in the above Reference Example 1.01 (2).

### Reference Examples 1.09 to 1.10

The compounds of Reference Examples 1.09 to 1.10 listed in the Table of Reference Examples as described hereinafter were obtained in the same manner as described in the above Reference Example 1.07.

### Reference Example 1.11

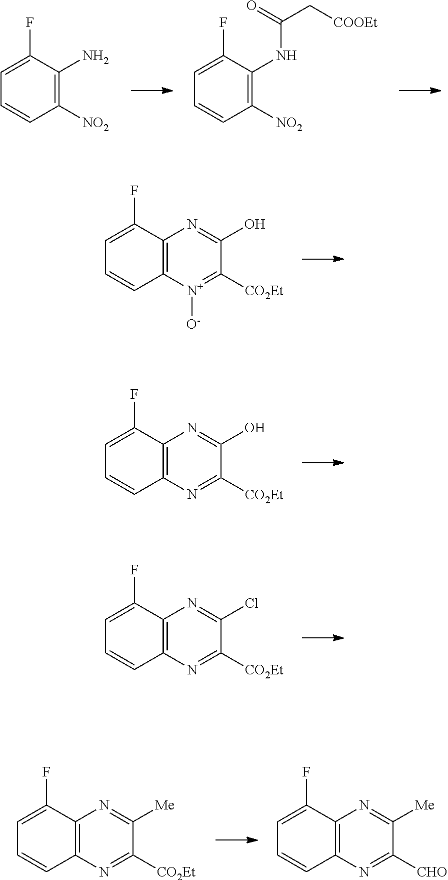

(1) The preparation was performed in the same manner as described in Bioorg. Med. Chem. 2005, 13, 5841 as in the following (1-i) to (1-v).

(1-i) To a solution of 2-fluoro-6-nitroaniline (20.0 g, 128 mmol) in toluene (250 mL) was added ethyl malonyl chloride (21.3 g, 141 mmol) at 0° C. After being refluxed for 3 hour, the reaction mixture was cooled to ambient temperature and diisopropyl ether was added. The precipitate was collected and washed with diisopropyl ether to give ethyl 3-[(2-fluoro-6-nitrophenyl)amino]-3-oxopropanoate as a pale brown powder (29.2 g, 84%). mp 99-102° C. MS (APCI): m/z 381 (M+H).

(1-ii) To a solution of ethyl 3-[(2-fluoro-6-nitrophenyl)amino]-3-oxopropanoate (10.0 g, 37.0 mmol) in N,N-dimethylformamide (50 mL) was added potassium tert-butoxide (8.31 g, 74.0 mmol) in N,N-dimethylformamide (50 mL) in one portion at 0° C. The reaction mixture was stirred for 15 min at 0° C., and then aqueous hydrogen chloride (6N) was added. The mixture was extracted with chloroform (400 mL). The organic layer was dried over magnesium sulfate, filtrated and concentrated in vacuo. The residue was purified by trituration with hexane-diisopropyl ether to give ethyl 5-fluoro-3-hydroxyquinoxaline-2-carboxylate 1-oxide as a pale brown powder (7.00 g, 75%). MS (APCI): m/z 253 (M+H).

(1-iii) A solution of ethyl 5-fluoro-3-hydroxyquinoxaline-2-carboxylate 1-oxide (7.00 g, 27.8 mmol) and phosphorus tribromide (7.70 mL, 83.3 mmol) in N,N-dimethylformamide (85 mL) was stirred for 45 min at room temperature. The reaction mixture was poured into cold water, and the mixture was extracted with ethyl acetate. The organic layer was washed with water, dried over magnesium sulfate, filtrated and concentrated in vacuo. The residue was purified by trituration with diisopropyl ether to give ethyl 5-fluoro-3-hydroxyquinoxaline-2-carboxylate as a pale yellow powder (4.60 g, 70%). MS (APCI): m/z 237 (M+H).

(1-iv) The mixture of ethyl 5-fluoro-3-hydroxyquinoxaline-2-carboxylate (11.4 g, 48.2 mmol) and phosphorus(V) oxychloride (37.0 g, 241 mmol) was heated for 3 hour at 115° C. After being cooled to ambient temperature, the reaction mixture was poured into cold water and extracted with ethyl acetate. The organic layer was washed with saturated sodium bicarbonate, dried over magnesium sulfate, filtrated and concentrated in vacuo. The residue was purified by silica gel column chromatography (hexane:ethyl acetate=50:1 to 9:1) to give ethyl 3-chloro-5-fluoroquinoxaline-2-carboxylate as a colorless solid (8.80 g, 72%). MS (APCI): m/z 255/257 (M+H).

(1-v) A suspension of ethyl 3-chloro-5-fluoroquinoxaline-2-carboxylate (8.80 g, 34.6 mmol), trimethylboroxine (8.68 g, 69.1 mmol), [1,1′-bis(diphenylphosphino)ferrocene]dichloropalladium(II) (1.41 g, 1.73 mmol), and potassium carbonate (11.9 g, 86.4 mmol) in 1,4-dioxane (200 mL) was heated for 14 hour at 115° C. After being cooled to ambient temperature, the reaction mixture was filtrated through celite with ethyl acetate. The filtrate was combined and concentrated in vacuo. The residue was purified by silica gel column chromatography (hexane:ethyl acetate=19:1 to 4:1) to give ethyl 5-fluoro-3-methylquinoxaline-2-carboxylate as colorless solid (8.02 g, 99%). mp 87-89° C. MS (APCI): m/z 235 (M+H).

(2) The preparation was performed in the same manner as described in Reference Example 1.01 (2) using ethyl 5-fluoro-3-methyl quinoxaline-2-carboxylate (4.00 g, 17.1 mmol) to give 5-fluoro-3-methylquinoxaline-2-carbaldehyde (the compound of Reference Example 1.11 listed in the Table of Reference Examples as described hereinafter) as a pale orange solid (2.14 g, 66%).

### Reference Examples 1.12

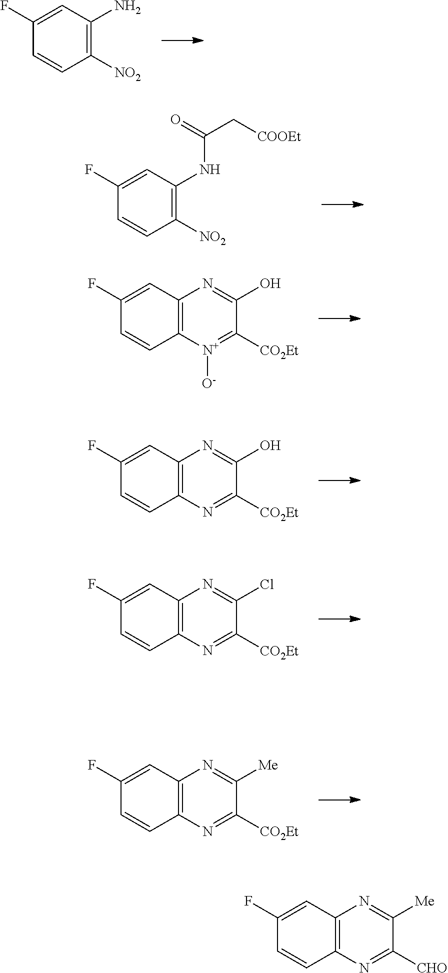

(1) The preparation was performed in the same manner as described in Bioorg. Med. Chem. 2005, 13, 5841 as in the following (1-i) to (1-v).

(1-i) To a solution of 5-fluoro-2-nitroaniline (25.0 g, 160 mmol) in toluene (320 mL) was added ethyl malonyl chloride (26.5 g, 176 mmol) at 0° C. After being refluxed for 2 hour, the reaction mixture was cooled to ambient temperature and diisopropyl ether was added. The precipitate was collected and washed with diisopropyl ether to give ethyl 3-[(5-fluoro-2-nitrophenyl)amino]-3-oxopropanoate as pale yellow powder (43.0 g, 99%). MS (APCI): m/z 271 (M+H).

(1-ii) To a solution of ethyl 3-[(5-fluoro-2-nitrophenyl)amino]-3-oxopropanoate (20.0 g, 74.0 mmol) in N,N-dimethylformamide (106 mL) was added potassium tert-butoxide (16.2 g, 144 mmol) in N,N-dimethylformamide (70 mL) in one portion at 0° C. The reaction mixture was stirred for 5 min at 0° C., and then aqueous potassium phosphate was added. The mixture was extracted with ethyl acetate. The organic layer was dried over sodium sulfate, filtrated and concentrated in vacuo. The residue was purified by trituration with chloroform to give ethyl 6-fluoro-3-hydroxyquinoxaline-2-carboxylate 1-oxide as orange powder (6.82 g, 37%). MS (APCI): m/z 253 (M+H).

(1-iii) A solution of ethyl 6-fluoro-3-hydroxyquinoxaline-2-carboxylate 1-oxide (9.09 g, 36.0 mmol) and phosphorus tribromide (6.77 mL, 72.1 mmol) in N,N-dimethylformamide (109 mL) was stirred for 30 min at room temperature. The reaction mixture was poured into cold water, and the mixture was extracted with chloroform. The organic layer was dried over sodium sulfate, filtrated and concentrated in vacuo. The residue was purified by trituration with diethyl ether to give ethyl 6-fluoro-3-hydroxyquinoxaline-2-carboxylate as pale yellow powder (5.70 g, 67%). MS (APCI): m/z 237 (M+H).

(1-iv) The mixture of ethyl 6-fluoro-3-hydroxyquinoxaline-2-carboxylate (5.70 g, 24.1 mmol) and phosphorus(V) oxychloride (37.0 g, 241 mmol) was heated for 2 hour at 115° C. After being cooled to ambient temperature, the reaction mixture was concentrated in vacuo. The residue was poured into saturate aqueous sodium bicarbonate and extracted with ethyl acetate. The organic layer was dried over sodium sulfate, filtrated and concentrated in vacuo. The residue was purified by silica gel column chromatography (hexane to hexane:ethyl acetate=9:1) to give ethyl 3-chloro-6-fluoroquinoxaline-2-carboxylate as colorless solid (3.72 g, 61%). MS (APCI): m/z 255/257 (M+H).

(1-v) A suspension of ethyl 3-chloro-6-fluoroquinoxaline-2-carboxylate (3.72 g, 14.6 mmol), trimethylboroxine (3.67 g, 29.2 mmol), [1,1′-bis(diphenylphosphino)ferrocene]dichloropalladium(II) (592 mg, 0.730 mmol), and potassium carbonate (5.05 g, 36.5 mmol) in 1,4-dioxane (97 mL) was heated for 3 hour at 115° C. After being cooled to ambient temperature, the reaction mixture was filtrated through celite with ethyl acetate. The filtrate was combined and concentrated in vacuo. The residue was purified by silica gel column chromatography (hexane to hexane:ethyl acetate=17:3) to give ethyl 6-fluoro-3-methylquinoxaline-2-carboxylate as colorless solid (2.67 g, 78%). MS (APCI): m/z 235 (M+H).

(2) To a solution of ethyl 6-fluoro-3-methylquinoxaline-2-carboxylate (1.60 g, 6.83 mmol) in tetrahydrofuran was added diisobutylaluminium hydride (0.99 M solution in toluene, 20.7 mL, 20.5 mmol) at −78° C. The reaction mixture was stirred for 1 hour at −78° C., and then methanol was added and allowed to warm to room temperature. The precipitate was removed through celite. The filtrate was combined and concentrated in vacuo. The residue was purified by silica gel column chromatography (hexane:ethyl acetate=19:1 to 4:1) to give 6-fluoro-3-methylquinoxaline-2-carbaldehyde (the compound of Reference Example 1.12 listed in the Table of Reference Examples as described hereinafter) as pale yellow solid (866 mg, 67%).

### Reference Example 1.13

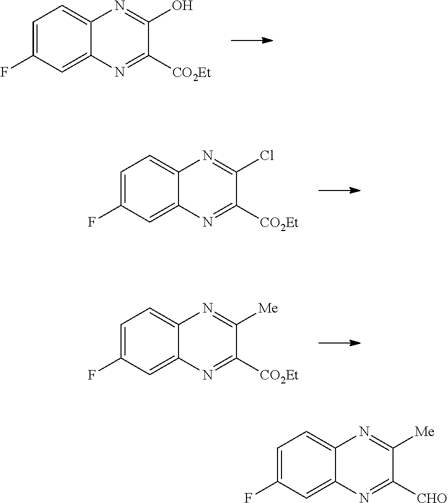

(1) A mixture of ethyl 7-fluoro-3-hydroxyquinoxaline-2-carboxylate (6.48 g, 27.4 mmol), referred to Bioorg. Med. Chem. 2005, 13, 5841-5863, and phosphorus(V) oxychloride (25.7 g, 168 mmol) was heated at 100° C. for 1 hour. After being cooled to an ambient temperature, the reaction mixture was concentrated in vacuo. The residue was poured into cold water (1000 mL) and extracted with ethyl acetate. The organic layer was washed with aqueous saturated sodium bicarbonate, dried over magnesium sulfate, filtrated and concentrated in vacuo to give ethyl 3-chloro-7-fluoroquinoxaline-2-carboxylate as a pale brown powder (6.78 g, 97%). MS (APCI): m/z 255/257 (M+H).

(2) A suspension of ethyl 3-chloro-7-fluoroquinoxaline-2-carboxylate (6.78 g, 26.6 mmol), trimethylboroxine (6.68 g, 53.2 mmol), [1,1′-bis(diphenylphosphino)ferrocene]dichloro-palladium(II), complex with dichloromethane (1:1) (1.09 g, 1.33 mmol), and potassium carbonate (9.20 g, 66.6 mmol) in 1,4-dioxane (150 mL) was heated at 115° C. for 1 hour. After being cooled to ambient temperature, the reaction mixture was filtrated through celite with ethyl acetate. The filtrate was combined and concentrated in vacuo. The residue was purified by silica gel column chromatography (hexane:ethyl acetate=19:1 to 9:1) to give ethyl 7-fluoro-3-methylquinoxaline-2-carboxylate as a colorless solid (5.83 g, 94%). MS (APCI): m/z 235 (M+H).

(3) To a solution of ethyl 7-fluoro-3-methylquinoxaline-2-carboxylate (5.83 g, 24.9 mmol) in tetrahydrofuran (250 mL) was added diisobutylaluminium hydride (0.99 M solution in toluene, 75.4 mL, 74.6 mmol) dropwise over 15 min at −78° C. The reaction mixture was stirred at the same temperature for 1.5 hour, and then methanol (25 mL) was added and followed by addition of aqueous saturated potassium sodium tartrate (300 mL). The mixture was allowed to warm to room temperature and extracted with diethyl ether (300 mL). The organic layer was dried over magnesium sulfate, filtrated and concentrated in vacuo. The residue was purified by silica gel column chromatography (hexane:ethyl acetate=4:1 to chloroform:ethyl acetate=9:1) to give 7-fluoro-3-methylqunoxaline-2-carbaldehyde (the compound of Reference Example 1.13 listed in the Table of Reference Examples as described hereinafter) as a brown solid (4.71 g, 99%). ¹H NMR (CDCl₃): δ 3.03 (3H, s), 7.68 (1H, ddd, J=2.7, 8.0, 9.2 Hz), 7.83 (1H, dd, J=2.7, 8.8 Hz), 8.10 (1H, dd, J=5.7, 9.4 Hz), 10.31 (1H, s).

### Reference Examples 1.14 to 1.17

The compound of Reference Examples 1.14 to 1.17 listed in the Table of Reference Examples as described hereinafter were obtained in the same manner as described in the above Reference Example 1.11.

### Reference Example 1.18

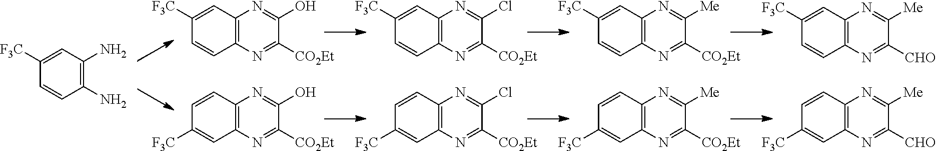

(1) The preparation was performed in the same manner as described in Bioorg. Med. Chem. 2006, 14, 776 using 3,4-diaminobenzenetrifluoride (2.72 g, 15.4 mmol) and diethyl ketomalonate (2.82 g, 16.2 mmol) to give ethyl 3-hydroxy-6-trifluoromethylquinoxaline-2-carboxylate as yellow solid (2.44 g, 55%) and ethyl 3-hydroxy-7-trifluoromethylquinoxaline-2-carboxylate as pale yellow solid (1.26 g, 11%).

Ethyl 3-hydroxy-6-trifluoromethylquinoxaline-2-carboxylate: MS (APCI): m/z 287 (M+H). ¹H-NMR (DMSO-d₆): δ 13.09 (1H, br), 8.05 (1H, d), 7.66-7.68 (1H, m), 7.63 (1H, br), 4.40 (2H, q), 1.37 (3H, t).

Ethyl 3-hydroxy-7-trifluoromethylquinoxaline-2-carboxylate: MS (APCI): m/z 287 (M+H). ¹H-NMR (DMSO-d₆): δ 13.16 (1H, br), 8.19 (1H, s), 7.96 (1H, dd), 7.51 (1H, d), 4.39 (2H, q), 1.33 (3H, t).

(2) The preparation was performed in the same manner as described in Reference Example 1.11 (1-iv) using ethyl 3-hydroxy-6-trifluoromethylquinoxaline-2-carboxylate (2.19 g, 7.29 mmol) to give ethyl 3-chloro-6-trifluoromethylquinoxaline-2-carboxylate as a pale pink oil (2.19 g, 99%). ¹H-NMR (CDCl₃): δ 8.38 (1H, br), 8.32 (1H, d), 8.02 (1H, dd), 4.59 (2H, q), 1.50 (3H, t). MS (APCI): m/z 301, 271.

Separately, the preparation was performed in the same manner as described in Reference Example 1.11 (1-iv) using ethyl 3-hydroxy-7-trifluoromethylquinoxaline-2-carboxylate (2.29 g, 8.02 mmol) to give ethyl 3-chloro-7-trifluoromethylquinoxaline-2-carboxylate as a brown oil (2.42 g, 99%). ¹H-NMR (CDCl₃): δ 8.51 (1H, br), 8.22 (1H, d), 8.06 (1H, dd), 4.59 (2H, q), 1.50 (3H, t). MS (APCI): m/z 301, 287, 271.

(3) The preparation was performed in the same manner as described in Reference Example 1.06 (1B) using ethyl 3-chloro-6-trifluoromethylquinoxaline-2-carboxylate (2.19 g, 7.19 mmol) to give ethyl 3-methyl-6-trifluoromethylquinoxaline-2-carboxylate as a pale yellow powder (1.95 g, 95%). MS (APCI): m/z 285 (M+H).

Separately, the preparation was performed in the same manner as described in Reference Example 1.06 (1B) using ethyl 3-chloro-7-trifluoromethylquinoxaline-2-carboxylate (2.42 g, 7.93 mmol) to give ethyl 3-methyl-7-trifluoromethylquinoxaline-2-carboxylate as a pale yellow solid (2.04 g, 89%). MS (APCI): m/z 285 (M+H).

(4) The preparation was performed in the same manner as described in Reference Example 1.01 (2) using ethyl 3-methyl-6-trifluoromethylquinoxaline-2-carboxylate (1.94 g, 6.83 mmol) to give 3-methyl-6-trifluoromethylquinoxaline-2-carbaldehyde (the compound of Reference Example 1.18(a) listed in the Table of Reference Examples as described hereinafter) as an orange oil (965 mg, 59%).

Separately, the preparation was performed in the same manner as described in Reference Example 1.01 (2) using ethyl 3-methyl-7-trifluoromethylquinoxaline-2-carboxylate (2.03 g, 7.16 mmol) to give 3-methyl-7-trifluoromethylquinoxaline-2-carbaldehyde (the compound of Reference Example 1.18(b) listed in the Table of Reference Examples as described hereinafter) as an orange solid (1.20 g, 70%).

### Reference Example 1.19

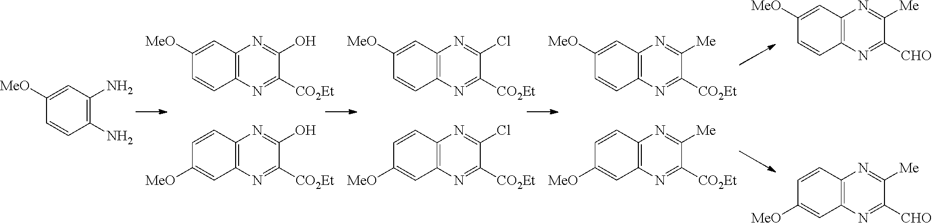

(1) A suspension of 4-methoxy-1,2-phenylenediamine dihydrochloride (2.0 g, 9.47 mmol) and diethyl ketomalonate (1.54 mL, 9.97 mmol), and triethylamine (2.64 mL, 18.9 mmol) in ethanol was refluxed for 1 hour. After being cooled to ambient temperature, the reaction mixture was concentrated in vacuo. The residue was triturated with hexane-diiopropyl ether to give a mixture of ethyl 3-hydroxy-6-methoxyquinoxaline-2-carboxylate and ethyl 3-hydroxy-7-methoxyquinoxaline-2-carboxylate as a colorless powder (4.50 g). MS (APCI): m/z 249 (M+H).

(2) A mixture of ethyl 3-hydroxy-6-methoxyquinoxaline-2-carboxylate and ethyl 3-hydroxy-7-methoxyquinoxaline-2-carboxylate (4.50 g) was treated with phosphorus(V) oxychloride according to the conditions described in Reference Example 1.11 (1-iv) to give a mixture of ethyl 3-chloro-6-methoxyquinoxaline-2-carboxylate and ethyl 3-chloro-7-methoxyquinoxaline-2-carboxylate as a yellow solid (2.02 g, 81%). MS (APCI): m/z 267/269 (M+H).

(3) A mixture of ethyl 3-chloro-6-methoxyquinoxaline-2-carboxylate and ethyl 3-chloro-7-methoxyquinoxaline-2-carboxylate (2.02 g) was treated with trimethylboroxine as described in Reference Example 1.11 (1-v) to give ethyl 6-methoxy-3-methylquinoxaline-2-carboxylate and ethyl 7-methoxy-3-methylquinoxaline-2-carboxylate.

The mixture was purified by medium pressure liquid chromatography (column: YAMAZEN, ULTRAPACK 40C, elution: hexane:ethyl acetate=4:1, flow rate: 15 mL/min) to give ethyl 6-methoxy-3-methylquinoxaline-2-carboxylate as colorless powder (701 mg) and ethyl 7-methoxy-3-methylquinoxaline-2-carboxylate as a colorless powder (889 mg).

Ethyl 6-methoxy-3-methylquinoxaline-2-carboxylate: ¹H-NMR (CDCl₃): δ 8.06 (1H, d), 7.40 (1H, dd), 7.32 (1H, d), 4.55 (2H, q), 3.98 (3H, s), 2.96 (3H, s), 1.49 (3H, t). MS (APCI): m/z 247 (M+H).

Ethyl 7-methoxy-3-methylquinoxaline-2-carboxylate: ¹H-NMR (CDCl₃): δ 7.93 (1H, dd), 7.49 (1H, d), 7.46 (1H, s), 4.56 (2H, q), 3.96 (3H, s), 2.92 (3H, s), 1.49 (3H, t). MS (APCI): m/z 247 (M+H).

(4) The preparation was performed in the same manner as described in Reference Example 1.01 (2) using ethyl 6-methoxy-3-methylquinoxaline-2-carboxylate (1.20 g, 4.87 mmol) to give 6-methoxy-3-methylquinoxaline-2-carbaldehyde (the compound of Reference Example 1.19 (a) listed in the Table of Reference Examples as described hereinafter) as yellow powder (775 mg, 79%).

Separately, the preparation was performed in the same manner as described in Reference Example 1.01 (2) using ethyl 7-methoxy-3-methylquinoxaline-2-carboxylate (885 mg, 3.59 mmol) to give 7-methoxy-3-methylquinoxaline-2-carbaldehyde (the compound of Reference Example 1.19 (b) listed in the Table of Reference Examples as described hereinafter) as a yellow powder (672 mg, 93%).

### Reference Example 1.20

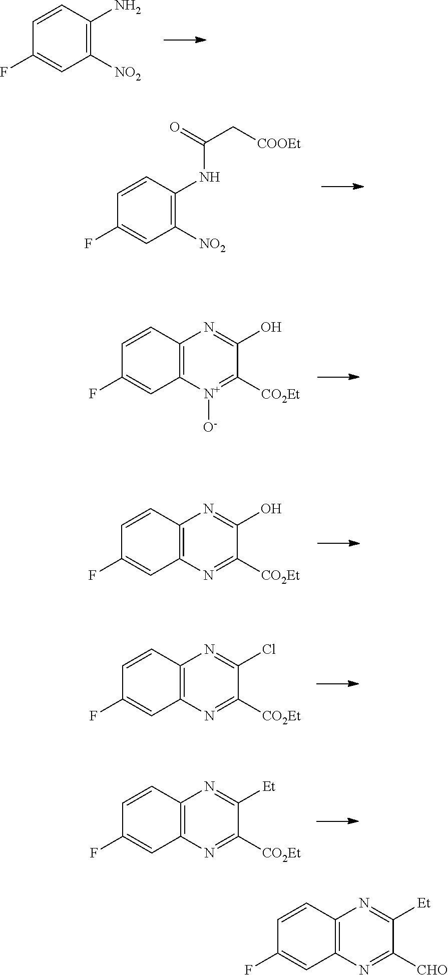

(1) The preparation was performed in the same manner as described in Bioorg. Med. Chem. 2005, 13, 5841 and Reference Example 1.11 (1-i) to (1-iv) starting with 4-fluoro-6-nitroaniline to give ethyl 3-chloro-7-fluoroquinoxaline-2-carboxylate. MS (APCI): m/z 255/257 (M+H).

(2) A suspension of ethyl 3-chloro-7-fluoroquinoxaline-2-carboxylate (2.00 g, 7.85 mmol), ethylboronic acid (2.03 g, 27.5 mmol), [1,1′-bis(diphenylphosphino)ferrocene]dichloropalladium(II) (641 mg, 0.785 mmol), and potassium carbonate (4.34 g, 31.4 mmol) in 1,4-dioxane (230 mL) was heated for 24 hour at 115° C. After being cooled to ambient temperature, the reaction mixture was filtrated through celite with ethyl acetate. The filtrate was combined and concentrated in vacuo. The residue was diluted with ethyl acetate and washed with water. The organic layer was dried over magnesium sulfate, filtrated and concentrated in vacuo. The residue was purified by silica gel column chromatography (hexane to hexane:ethyl acetate=4:1) to give ethyl 3-ethyl-7-fluoroquinoxaline-2-carboxylate as colorless solid (1.33 g, 68%). mp 42-45° C. MS (APCI): m/z 249 (M+H).

(3) Preparation was performed in the same manner as described in Reference Example 1.01 (2) using ethyl 3-ethyl-7-fluoroquinoxaline-2-carboxylate (1.32 g, 5.32 mmol) to give 3-ethyl-7-fluoroquinoxaline-2-carbaldehyde (the compound of Reference Example 1.20 listed in the Table of Reference Examples as described hereinafter) as yellow powder (1.29 g, quant.).

### Reference Example 2.01

The preparation was performed in the same manner as described in WO 2005/042533 to give 4-methyl-4-aminotetrahydro-2H-pyran hydrochloride (the compound of Reference Example 2.01 listed in the Table of Reference Examples as described hereinafter).

### Reference Example 2.02

The preparation was performed in the same manner as described in WO2007/046548 to give (3R)-1,1-dioxidotetrahydro-3-thienylamine hydrochloride (the compound of Reference Example 2.02 listed in the Table of Reference Examples as described hereinafter).

### Reference Example 2.03

The preparation was performed in the same manner as described in WO2007/046548 to give (3S)-1,1-dioxidotetrahydro-3-thienylamine hydrochloride (the compound of Reference Example 2.03 listed in the Table of Reference Examples as described hereinafter).

### Reference Example 2.04

The preparation was performed in the same manner as described in JP2006-67705 and JP2007-62718 to give trans-4-amino-1-methylcyclohexanol (the compound of Reference Example 2.04 listed in the Table of Reference Examples as described hereinafter).

### Reference Example 2.05

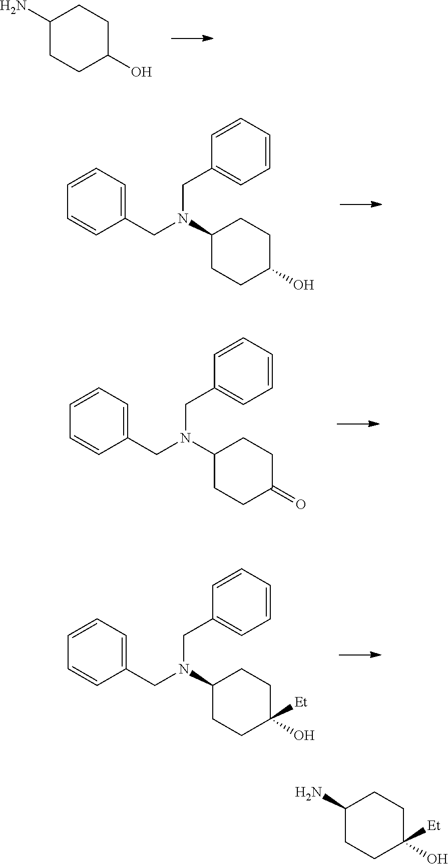

(1) A suspension of 4-aminocyclohexanol (11.5 g, 100 mmol), benzylbromide (34.2 g, 200 mmol), tetrabutylammonium iodide (3.69 g, 10.0 mmol), and sodium carbonate (21.2 g, 200 mmol) in tetrahydrofuran (200 mL) was refluxed for 17 hour. After being cooled to ambient temperature, the reaction mixture was concentrated in vacuo. The residue was purified by trituration with diethyl ether-diisopropyl ether to give trans-4-(dibenzylamino)cyclohexanol as a colorless powder (21.4 g, 72%). MS (APCI): m/z 296 (M+H).

(2) To a solution of oxalyl chloride (6.28 mL, 72.0 mmol) in dichloromethane (200 mL) was added dimethylsulfoxide (10.7 mL, 150 mmol) in dichloromethane (100 mL) at −78° C. After being stirred for 20 min at −78° C., a solution of trans-4-(dibenzylamino)cyclohexanol (17.7 g, 60.0 mmol) was added. The reaction mixture was stirred for 35 min at −78° C., and then triethylamine (43.9 mL, 315 mmol) was added. After being warmed to room temperature, the reaction mixture was poured into water (400 mL). The mixture was extracted with chloroform. The organic layer was washed with saturated brine, dried over magnesium sulfate, filtrated and concentrated in vacuo. The residue was purified by silica gel column chromatography (hexane-ethyl acetate=4:1) to give 4-(dibenzylamino)cyclohexan-1-one as a colorless powder (16.9 g, 96%). MS (APCI): m/z 294 (M+H).

(3) To a solution of triethylaluminium (1.0M in hexane, 66.0 mL, 66.0 mmol) in toluene (132 mL) was added dropwise a solution of 4-(dibenzylamino)cyclohexan-1-one (8.80 g, 30.0 mmol) over 15 min at room temperature. After being stirred for 30 min at room temperature, aqueous sodium hydroxide (2N, 37.5 mL, 75 mmol) was added, and the organic layer was separated. The organic layer was washed with water and saturated brine, dried over magnesium sulfate, filtrated and concentrated in vacuo. The residue was purified by silica gel column chromatography (hexane:ethyl acetate=4:1) to give trans-4-(dibenzylamino)-1-ethylcyclohexanol as a colorless solid (6.63 g, 68%). MS (APCI): m/z 324 (M+H).

(4) A suspension of trans-4-(dibenzylamino)-1-ethylcyclohexanol (6.20 g, 19.2 mmol) and palladium on carbon (5%, 5.0 g) in methanol was stirred for 21 hour under hydrogen atmosphere. The reaction mixture was filtrated and concentrated in vacuo. The residue was purified by trituration with diethyl ether to give trans-4-amino-1-ethylcyclohexanol (the compound of Reference Example 2.05 listed in the Table of Reference Examples as described hereinafter) as a colorless solid (2.43 g, 89%).

### Reference Example 2.06

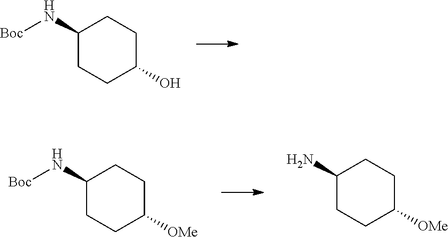

(1) To a solution of tert-butyl(trans-4-hydroxycyclohexyl)carbamate (1.08 g, 5.00 mmol) and 15-crown 5 (1.04 mL, 5.25 mmol) in tetrahydrofuran was added sodium hydride (60% dispersion in mineral oil, 440 mg, 11.0 mmol) at 0° C., followed by iodomethane (0.327 mL, 5.25 mmol) at 0° C. After being stirred for 2 hour, the reaction mixture was poured into water. The mixture was extracted with ethyl acetate. The organic layer was washed with water and saturated brine, died over sodium sulfate, filtrated and concentrated in vacuo. The residue was purified by silica gel column chromatography to give tert-butyl(trans-4-methoxycyclohexyl)carbamate as a colorless solid (796 mg, 69%). MS (APCI): m/z 247 (M+NH₄), 230 (M+H).

(2) To a solution of tert-butyl(trans-4-methoxycyclohexyl)carbamate (2.33 g, 10.2 mmol) in 1,4-dioxane (10 mL) was added hydrogen chloride in 1,4-dioxane (4N, 10.0 mL, 40.0 mmol) at 0° C. After being stirred for 20 hour, diethyl ether (100 mL) was added. The precipitate was collected and washed with diethyl ether to give trans-4-methoxycyclohexylamine hydrochloride (the compound of Reference Example 2.06 listed in the Table of Reference Examples as described hereinafter) as colorless crystals (1.54 g, 91%).

### Reference Example 2.07

The compound of Reference Example 2.07 listed in the Table of Reference Examples as described hereinafter was obtained in the same manner as described in the above Reference Example 2.06.

### Reference Example 2.08

The preparation was performed in the same manner as described in WO 96/07657 to give trans-4-hydroxymethylcyclohexylamine hydrochloride (the compound of Reference Example 2.08 listed in the Table of Reference Examples as described hereinafter).

### Reference Example 2.09

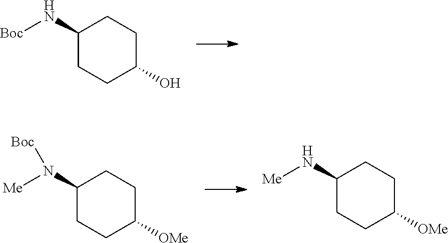

(1) A solution of tert-butyl(trans-4-hydroxycyclohexyl)carbamate (10.1 g, 46.9 mmol), sodium hydride (60% dispersion in mineral oil, 4.13 g, 103 mmol), and iodomethane (7.30 g, 51.6 mmol) in dimethylsulfoxide (0.94 mL) and tetrahydrofuran (47 mL) was heated at 70° C. for 8 hour, and then iodomethane (7.30 g, 51.6 mmol) was added. After being heated at 70° C. for 8 hour, the reaction mixture was poured into water. The mixture was extracted with ethyl acetate. The organic layer was washed with saturated brine, dried over magnesium sulfate, filtrated and concentrated in vacuo. The residue was purified by silica gel column chromatography (hexane:ethyl acetate=5:1) to give tert-butyl(trans-4-methoxycyclohexyl)methylcarbamate as colorless oil (5.19 g, 46%). MS (APCI): m/z 244 (M+H).

(2) The preparation was performed in the same manner as described in Reference Example 2.06 (2) using tert-butyl(trans-4-methoxycyclohexyl)methylcarbamate (5.18 g, 21.3 mmol) to give trans-4-methoxy-N-methylcyclohexylamine hydrochloride (the compound of Reference Example 2.09 listed in the Table of Reference Examples as described hereinafter) as colorless plates (3.36 g, 88%).

### Reference Examples 3.01 to 3.24

The compounds of Reference Examples 3.01 to 3.24 listed in the Table of Reference Examples as described hereinafter were obtained in the same manner as described in the above Example 1.001 (3), 1.048 (1), or 1.078 (1).

### Reference Example 3.25

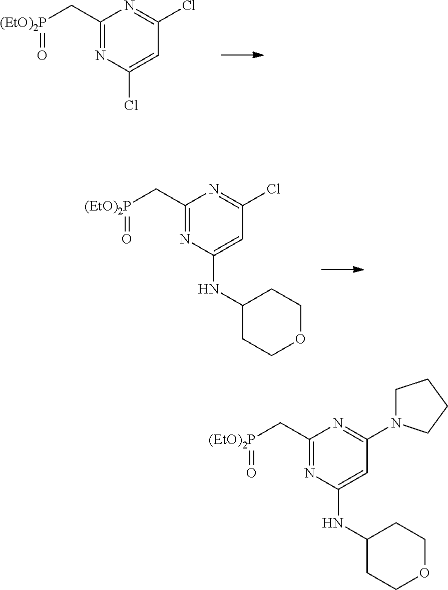

(1) A mixture of diethyl [(4,6-dichloropyrimidin-2-yl)methyl]phosphonate (539 mg, 1.80 mmol), 4-aminotetrahydro-2H-pyran acetate (640 mg, 3.97 mmol), and triethylamine (456 mg, 4.51 mmol) in N,N-dimethylformamide (15 mL) was stirred at room temperature for 40 hour. The reaction mixture was poured into saturated brine, and the mixture was extracted with ethyl acetate. The organic layer was washed with saturated brine, dried over sodium sulfate, filtrated and concentrated in vacuo. The residue was purified by silica gel column chromatography (chloroform to chloroform:methanol=19:1) to give diethyl {[4-chloro-6-(tetrahydro-2H-pyran-4-ylamino)pyrimidin-2-yl]methyl}phosphonate as a pale yellow oil (434 mg, 66%). MS (APCI): m/z 364/366 (M+H).

(2) A mixture of diethyl {[4-chloro-6-(tetrahydro-2H-pyran-4-ylamino)pyrimidin-2-yl]methyl}phosphonate (1.41 g, 3.86 mmol) and pyrrolidine (824 mg, 11.6 mmol) in N,N-dimethylacetamide (40 mL) was stirred at 65° C. for 3 days. After being cooled to an ambient temperature, the reaction mixture was poured into water, and the mixture was extracted with ethyl acetate. The organic layer was washed with water and saturated brine, dried over sodium sulfate, filtrated and concentrated in vacuo. The residue was purified by trituration with diethyl ether to give diethyl {[4-pyrrolidin-1-yl-6-(tetrahydro-2H-pyran-4-ylamino)pyrimidin-2-yl]methyl}phosphonate (the compound of Reference Example 3.25 listed in the Table of Reference Examples as described hereinafter) as a pink powder (1.04 g, 68%). ¹H NMR (CDCl₃): δ 1.31 (6H, t, J=6.8 Hz), 1.46-1.55 (2H, m), 1.93-1.97 (4H, m), 2.00 (2H, dd, J=13.0, 1.5 Hz), 3.23 (2H, d, J=21.8 Hz), 3.41 (4H, m), 3.51 (2H, td, J=11.5, 2.4 Hz), 3.64-3.72 (1H, m), 3.97 (2H, ddd, J=11.7, 3.9, 3.7 Hz), 4.12 (4H, m), 4.51 (1H, d, J=8.16 Hz), 5.03 (1H, s).

The structural formula and physical properties, etc. of the compounds of the Examples and the Reference Examples are shown in the following Tables and Tables of Reference Example.

In the tables, “MS (APCI)(m/z)” means mass spectrometry (Atmospheric pressure chemical ionization mass spectrometry). The “mp” means melting point. The following abbreviations are utilized in the Examples, Reference Examples and the following Tables:

“Me” means methyl group;
“Et” means ethyl group;
“Bu” means butyl group; and
“Boc” means tert-butoxycarbonyl group.

TABLE 1

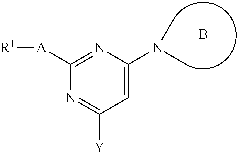

| Ex- ample No. | R¹—A— | 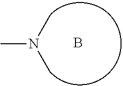 | —Y | Salt | Physical proper- ties, etc. |
|---|---|---|---|---|---|
| 1.001 | 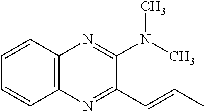 | 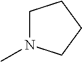 | 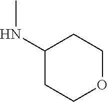 | 2HCl | MS (APCI): m/z 446 (M + H). |
| 1.002 | 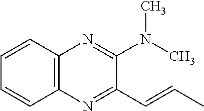 | 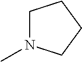 | 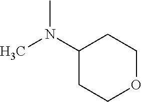 | 2HCl | MS (APCI): m/z 460 (M + H) |
| 1.003 | 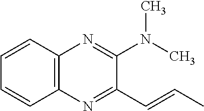 | 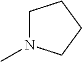 | 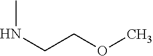 | 2HCl | MS (APCI): m/z 420 (M + H) |
| 1.004 | 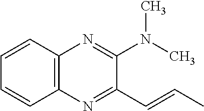 | 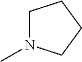 | 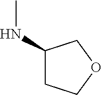 | 2HCl | MS (APCI): m/z 432 (M + H) |
| 1.005 | 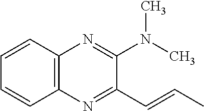 | 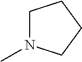 | 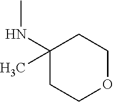 | 2HCl | MS (APCI): m/z 460 (M + H) |
| 1.006 | 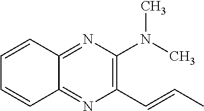 | 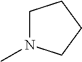 | 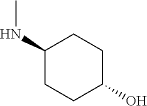 | 2HCl | MS (APCI): m/z 460 (M + H) |
| 1.007 | 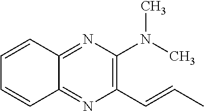 | 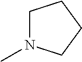 | 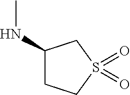 | 2HCl | MS (APCI): m/z 480 (M + H) |
| 1.008 | 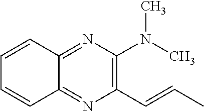 | 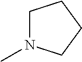 | 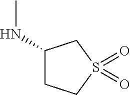 | 2HCl | MS (APCI): m/z 480 (M + H) |
| 1.009 | 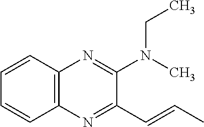 | 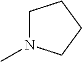 | 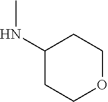 | 2HCl | MS (APCI): m/z 460 (M + H) |
| 1.010 | 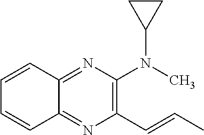 | 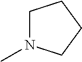 | 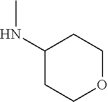 | 2HCl | MS (APCI): m/z 472 (M + H) |
| 1.011 | 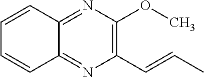 | 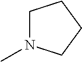 | 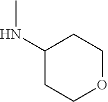 | free form | MS (APCI): m/z 433 (M + H) |
| 1.012 | 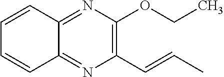 | 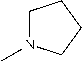 | 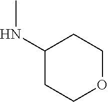 | HCl | MS (APCI): m/z 447 (M + H) |
| 1.013 | 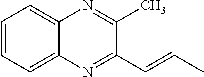 | 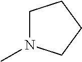 | 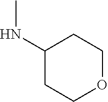 | 2HCl | MS (APCI): m/z 417 (M + H) |
| 1.014 | 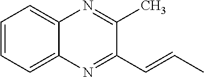 | 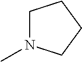 | 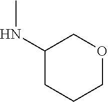 | 2HCl | MS (APCI): m/z 417 (M + H) |
| 1.015 | 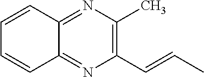 | 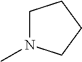 | 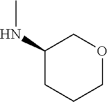 | 2HCl | MS (APCI): m/z 417 (M + H) |
| 1.016 | 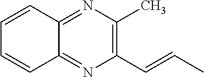 | 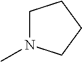 | 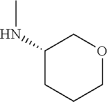 | 2HCl | MS (APCI): m/z 417 (M + H) |
| 1.017 | 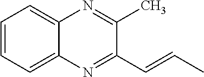 | 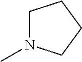 | 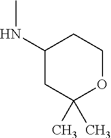 | 2HCl | MS (APCI): m/z 445 (M + H) |
| 1.018 | 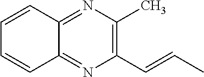 | 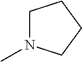 | 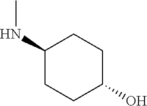 | 2HCl | MS (APCI): m/z 431 (M + H) |
| 1.019 | 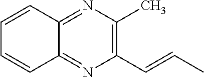 | 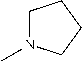 | 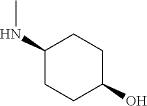 | 2HCl | MS (APCI): m/z 431 (M + H) |
| 1.020 | 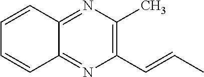 | 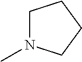 | 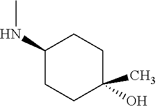 | 2HCl, HCl | MS (APCI): m/z 445 (M + H) |
| 1.021 | 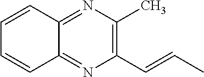 | 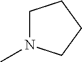 | 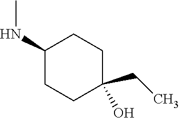 | 2HCl | MS (APCI): m/z 459 (M + H) |
| 1.022 | 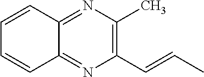 | 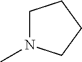 | 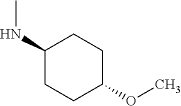 | 2HCl | MS (APCI): m/z 445 (M + H) |
| 1.023 | 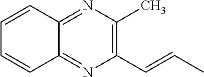 | 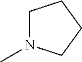 | 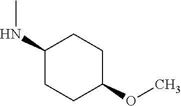 | 2HCl | MS (APCI): m/z 445 (M + H) |
| 1.024 | 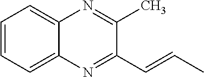 | 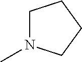 | 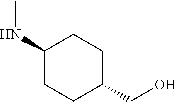 | 3/2HCl | MS (APCI): m/z 445 (M + H) |
| 1.025 | 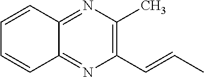 | 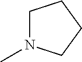 | 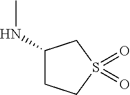 | 2HCl | MS (APCI): m/z 451 (M + H) |
| 1.026 | 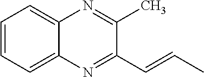 | 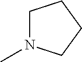 | 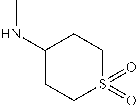 | 2HCl | MS (APCI): m/z 465 (M + H) |
| 1.027 | 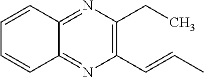 | 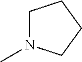 | 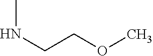 | 3/2HCl | MS (APCI): m/z 405 (M + H) |
| 1.028 | 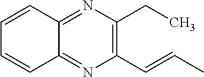 | 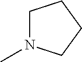 | 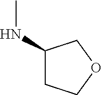 | 2HCl | MS (APCI): m/z 417 (M + H) |
| 1.029 | 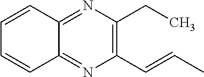 | 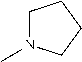 | 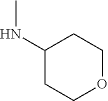 | 2HCl | MS (APCI): m/z 431 (M + H) |
| 1.030 | 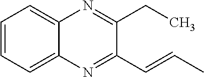 | 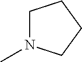 | 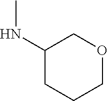 | HCl | MS (APCI): m/z 431 (M + H) |
| 1.031 | 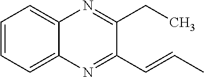 | 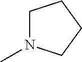 | 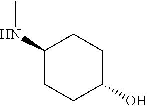 | 2HCl | MS (APCI): m/z 445 (M + H) |
| 1.032 | 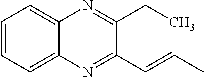 | 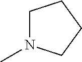 | 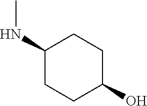 | 2HCl | MS (APCI): m/z 445 (M + H) |
| 1.033 | 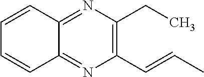 | 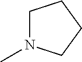 | 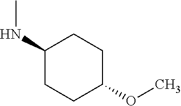 | 2HCl | MS (APCI): m/z 459 (M + H) |
| 1.034 | 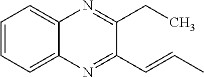 | 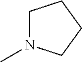 | 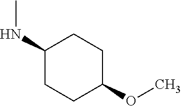 | HCl | MS (APCI): m/z 459 (M + H) |
| 1.035 | 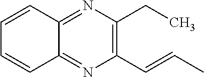 | 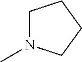 | 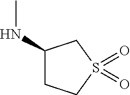 | 2HCl | MS (APCI): m/z 465 (M + H) |
| 1.036 | 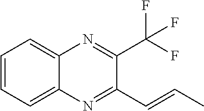 | 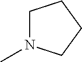 | 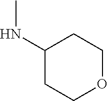 | HCl | MS (APCI): m/z 471 (M + H) |
| 1.037 | 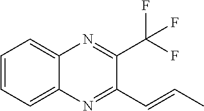 | 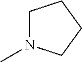 | 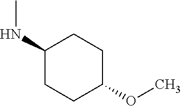 | HCl | MS (APCI): m/z 499 (M + H) |
| 1.038 | 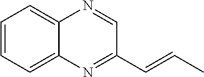 | 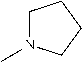 | 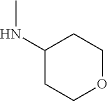 | free form | MS (APCI): m/z 403 (M + H) |
| 1.039 | 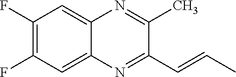 | 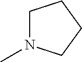 | 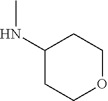 | 2HCl | MS (APCI): m/z 453 (M + H) |
| 1.040 | 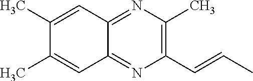 | 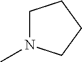 | 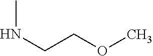 | 2HCl | MS (APCI): m/z 419 (M + H) |
| 1.041 | 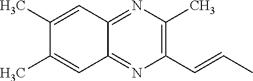 | 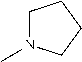 | 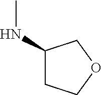 | 2HCl | MS (APCI): m/z 431 (M + H) |
| 1.042 | 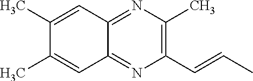 | 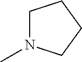 | 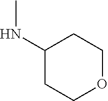 | 2HCl | MS (APCI): m/z 445 (M + H) |
| 1.043 | 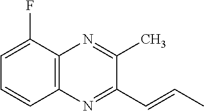 | 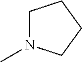 | 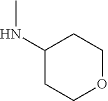 | HCl | MS (APCI): m/z 435 (M + H) |
| 1.044 | 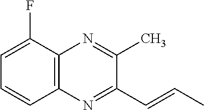 | 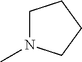 | 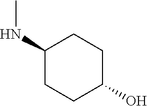 | HCl | MS (APCI): m/z 449 (M + H) |
| 1.045 | 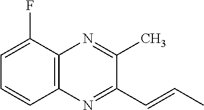 | 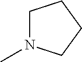 | 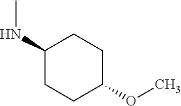 | HCl | MS (APCI): m/z 463 (M + H) |
| 1.046 | 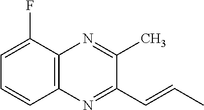 | 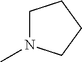 | 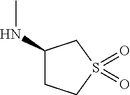 | HCl | MS (APCI): m/z 469 (M + H) |
| 1.047 | 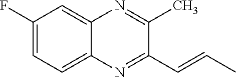 | 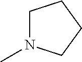 | 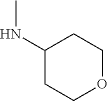 | 2HCl | MS (APCI): m/z 435 (M + H) |
| 1.048 | 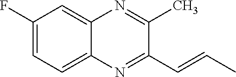 | 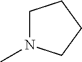 | 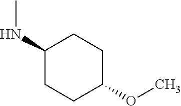 | 2HCl | MS (APCI): m/z 463 (M + H) |
| 1.049 | 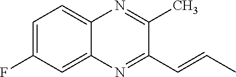 | 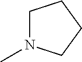 | 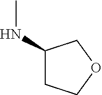 | 2HCl | MS (APCI): m/z 421 (M + H) |
| 1.050 | 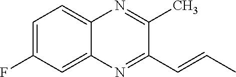 | 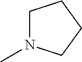 | 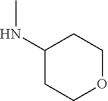 | HCl, 2HCl | MS (APCI): m/z 435 (M + H) |
| 1.051 | 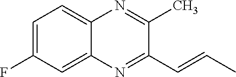 | 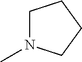 | 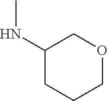 | 3/2HCl | MS (APCI): m/z 435 (M + H) |
| 1.052 | 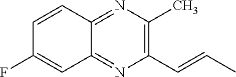 | 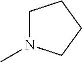 | 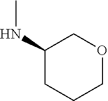 | 2HCl | MS (APCI): m/z 435 (M + H) |
| 1.053 | 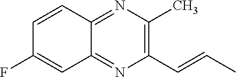 | 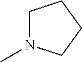 | 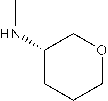 | 2HCl | MS (APCI): m/z 435 (M + H) |
| 1.054 | 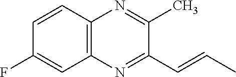 | 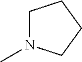 | 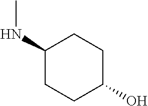 | 3/2HCl, 2HCl | MS (APCI): m/z 449 (M + H) |
| 1.055 | 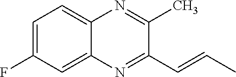 | 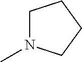 | 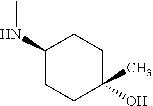 | 2HCl | MS (APCI): m/z 463 (M + H) |
| 1.056 | 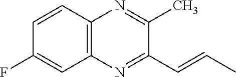 | 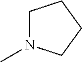 | 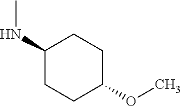 | 3/2HCl, HCl | MS (APCI): m/z 463 (M + H) |
| 1.057 | 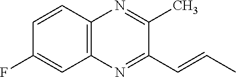 | 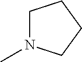 | 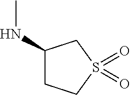 | 3/2HCl | MS (APCI): m/z 469 (M + H) |
| 1.058 | 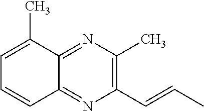 | 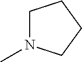 | 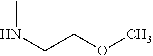 | 2HCl | MS (APCI): m/z 405 (M + H) |
| 1.059 | 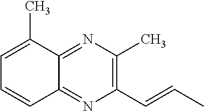 | 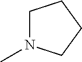 | 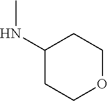 | 2HCl | MS (APCI): m/z 431 (M + H) |
| 1.060 | 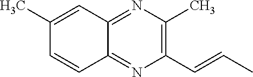 | 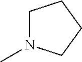 | 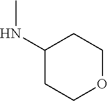 | 2HCl | MS (APCI): m/z 431 (M + H) |
| 1.061 | 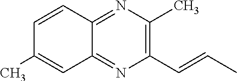 | 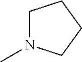 | 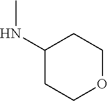 | 2HCl | MS (APCI): m/z 431 (M + H) |
| 1.062 | 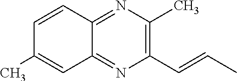 | 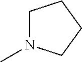 | 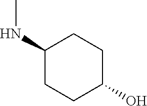 | 2HCl | MS (APCI): m/z 445 (M + H) |
| 1.063 | 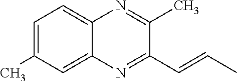 | 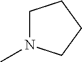 | 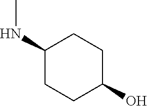 | 2HCl | MS (APCI): m/z 445 (M + H) |
| 1.064 | 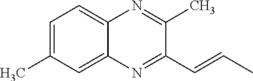 | 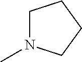 | 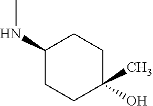 | 2HCl | MS (APCI): m/z 459 (M + H) |
| 1.065 | 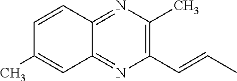 | 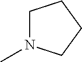 | 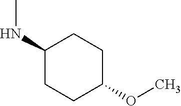 | 2HCl | MS (APCI): m/z 459 (M + H) |
| 1.066 | 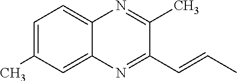 | 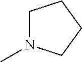 | 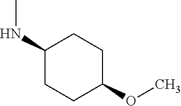 | 3/2HCl | MS (APCI): m/z 459 (M + H) |
| 1.067 | 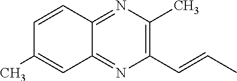 | 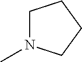 | 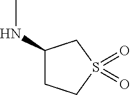 | 2HCl | MS (APCI): m/z 465 (M + H) |
| 1.068 | 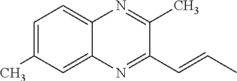 | 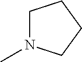 | 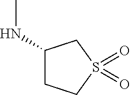 | 3/2HCl | MS (APCI): m/z 465 (M + H) |
| 1.069 | 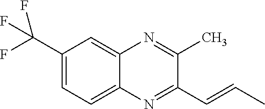 | 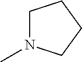 | 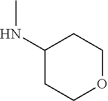 | free form | MS (APCI): m/z 485 (M + H) |
| 1.070 | 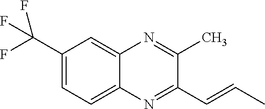 | 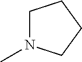 | 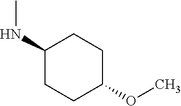 | free form | MS (APCI): m/z 513 (M + H) |
| 1.071 | 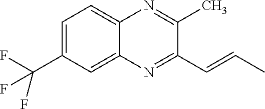 | 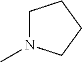 | 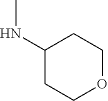 | free form | MS (APCI): m/z 485 (M + H) |
| 1.072 | 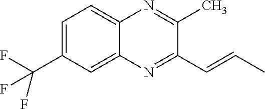 | 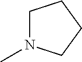 | 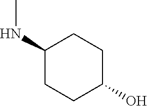 | free form | MS (APCI): m/z 499 (M + H) |
| 1.073 | 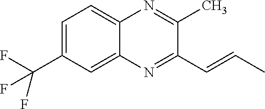 | 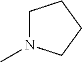 | 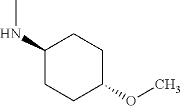 | free form | MS (APCI): m/z 513 (M + H) |
| 1.074 | 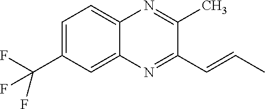 | 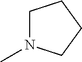 | 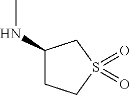 | free form | MS (APCI): m/z 519 (M + H) |
| 1.075 | 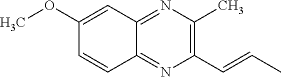 | 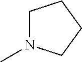 | 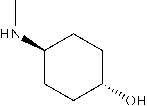 | 2HCl | MS (APCI): m/z 461 (M + H) |
| 1.076 | 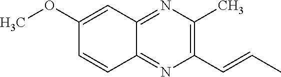 | 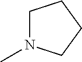 | 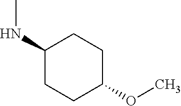 | 2HCl | MS (APCI): m/z 475 (M + H) |
| 1.077 | 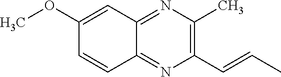 | 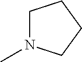 | 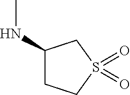 | 2HCl | MS (APCI): m/z 481 (M + H) |
| 1.078 | 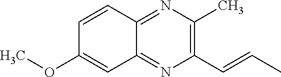 | 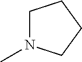 | 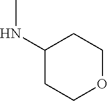 | 2HCl | MS (APCI): m/z 447 (M + H) |
| 1.079 | 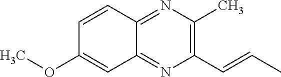 | 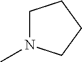 | 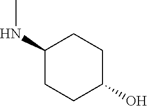 | 2HCl | MS (APCI): m/z 461 (M + H) |
| 1.080 | 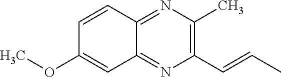 | 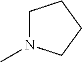 | 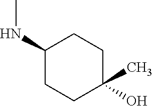 | 3/2HCl | MS (APCI): m/z 475 (M + H) |
| 1.081 | 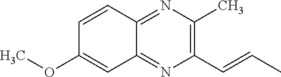 | 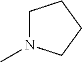 | 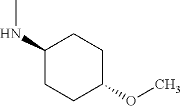 | 2HCl | MS (APCI): m/z 475 (M + H) |
| 1.082 | 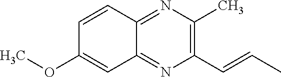 | 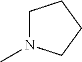 | 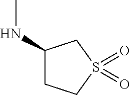 | 2HCl | MS (APCI): m/z 481 (M + H) |
| 1.083 | 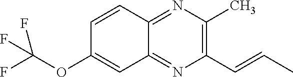 | 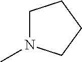 | 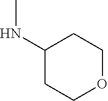 | 3/2HCl | MS (APCI): m/z 501 (M + H) |
| 1.084 | 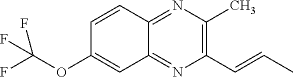 | 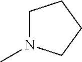 | 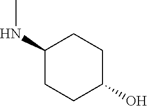 | 3/2HCl | MS (APCI): m/z 515 (M + H) |
| 1.085 | 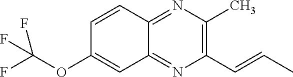 | 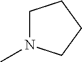 | 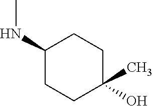 | 3/2HCl | MS (APCI): m/z 529 (M + H) |
| 1.086 | 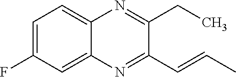 | 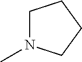 | 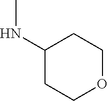 | 2HCl | MS (APCI): m/z 449 (M + H) |
| 1.087 | 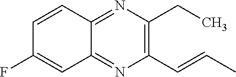 | 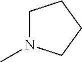 | 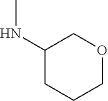 | 3/2HCl | MS (APCI): m/z 449 (M + H) |
| 1.088 | 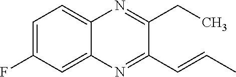 | 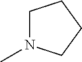 | 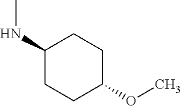 | 2HCl | MS (APCI): m/z 477 (M + H) |
| 1.089 | 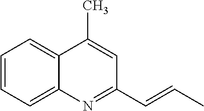 | 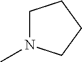 | 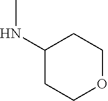 | 2HCl | MS (APCI): m/z 416 (M + H) |
| 1.090 | 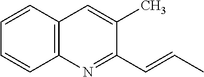 | 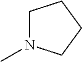 | 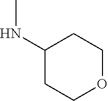 | 2HCl | MS (APCI): m/z 416 (M + H) |
| 1.091 | 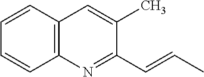 | 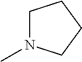 | 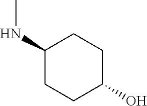 | 2HCl | MS (APCI): m/z 430 (M + H) |
| 1.092 | 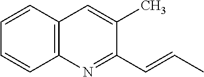 | 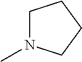 | 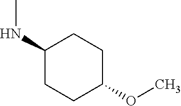 | 2HCl | MS (APCI): m/z 444 (M + H) |
| 1.093 | 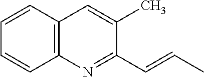 | 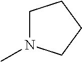 | 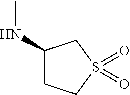 | 2HCl | MS (APCI): m/z 450 (M + H) |
| 1.094 | 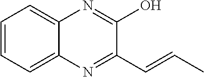 | 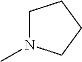 | 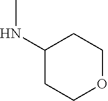 | free form, HCl | MS (APCI): m/z 419 (M + H) |
| 1.095 | 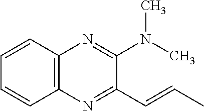 | 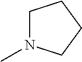 | 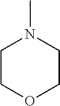 | 2HCl | MS (APCI): m/z 432 (M + H) |
| 1.096 | 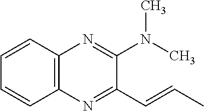 | 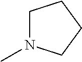 | 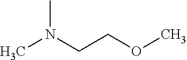 | 2HCl | MS (APCI): m/z 434 (M + H) |
| 1.097 | 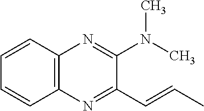 | 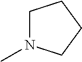 | 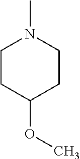 | HCl | MS (APCI): m/z 460 (M + H) |
| 1.098 | 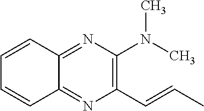 | 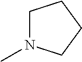 | 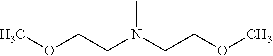 | 2HCl | MS (APCI): m/z 478 (M + H) |
| 1.099 | 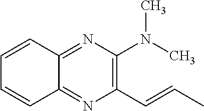 | 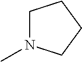 | 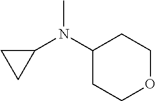 | 2HCl | MS (APCI): m/z 486 (M + H) |
| 1.100 | 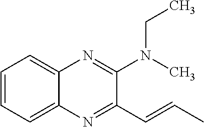 | 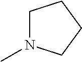 | 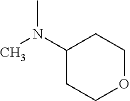 | 2HCl | MS (APCI): m/z 474 (M + H) |
| 1.101 | 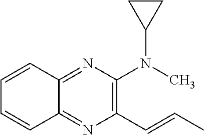 | 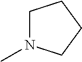 | 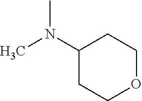 | 2HCl | MS (APCI): m/z 486 (M + H) |
| 1.102 | 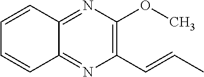 | 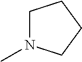 | 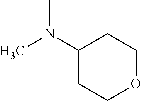 | 2HCl | MS (APCI): m/z 447 (M + H) |
| 1.103 | 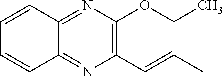 | 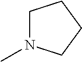 | 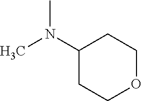 | 2HCl | MS (APCI): m/z 461 (M + H) |
| 1.104 | 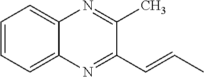 | 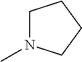 | 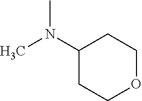 | 2HCl | MS (APCI): m/z 431 (M + H) |
| 1.105 | 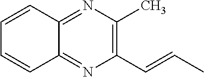 | 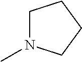 | 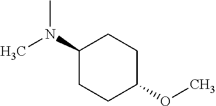 | 2HCl | MS (APCI): m/z 459 (M + H) |
| 1.106 | 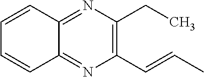 | 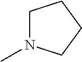 | 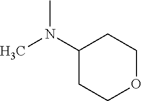 | 2HCl | MS (APCI): m/z 445 (M + H) |
| 1.107 | 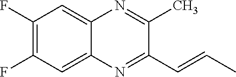 | 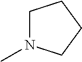 | 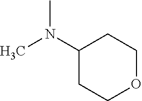 | HCl | MS (APCI): m/z 467 (M + H) |
| 1.108 | 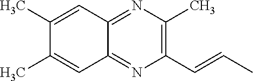 | 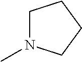 | 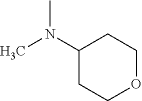 | 2HCl | MS (APCI): m/z 459 (M + H) |
| 1.109 | 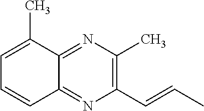 | 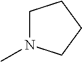 | 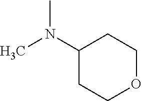 | 3/2HCl | MS (APCI): m/z 445 (M + H) |

TABLE 2

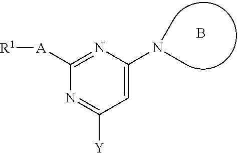

| Example No. | R¹—A— | 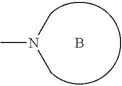 | —Y | Salt | Physical properties, etc. |
|---|---|---|---|---|---|
| 2.001 | 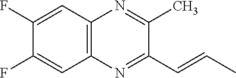 | 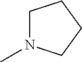 | 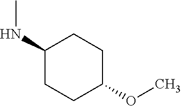 | 2HCl | MS (APCI): m/z 481 (M + H) |
| 3.001 | 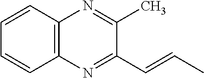 | 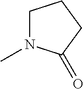 | 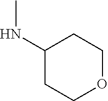 | HCl | MS (APCI): m/z 431 (M + H) |
| 4.001 | 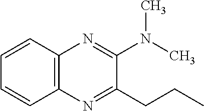 | 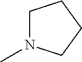 | 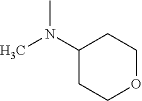 | free form | MS (APCI): m/z 462 (M + H) |
| 4.002 | 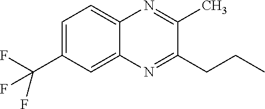 | 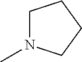 | 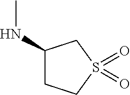 | free form | MS (APCI): m/z 521 (M + H) |
| 4.003 | 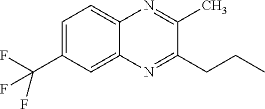 | 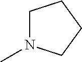 | 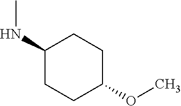 | free form | MS (APCI): m/z 515 (M + H) |
| 5.001 | 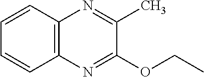 | 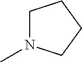 | 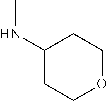 | 2HCl | MS (APCI): m/z 421 (M + H) |
| 5.002 | 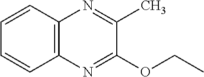 | 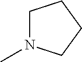 | 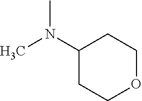 | HCl | MS (APCI): m/z 435 (M + H) |

TABLE 3

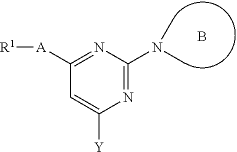

| Example No. | R¹—A— | 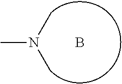 | —Y | Salt | Physical properties, etc. |
|---|---|---|---|---|---|
| 6.001 | 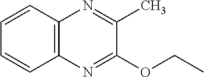 | 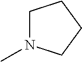 | 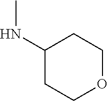 | HCl | MS (APCI): m/z 421 (M + H) |

Table of Reference Examples

| Reference | | | |
|---|---|---|---|
| Example No. | Structural formula | Salt | Physical properties, etc. |
| 1.01 | 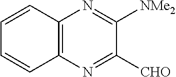 | free form | mp: 111-112° C. from hexane-diethyl ether. MS (APCI): m/z 204 (M + H). |
| 1.02 | 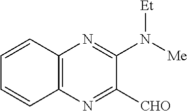 | free form | MS (APCI): m/z 216 (M + H). |
| 1.03 | 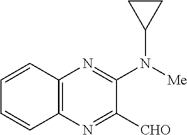 | free form | MS (APCI): m/z 228 (M + H) |
| 1.04 | 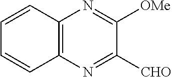 | free form | MS (APCI): m/z 189 (M + H) |
| 1.05 | 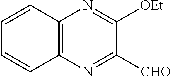 | free form | MS (APCI): m/z 203 (M + H) |
| 1.06 | 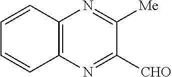 | free form | MS (APCI): m/z 173 (M + H) |
| 1.07 | 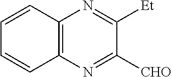 | free form | MS (APCI): m/z 187 (M + H) |
| 1.08 | 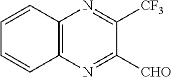 | free form | mp 122-123° C. MS (APCI): m/z 241 (M + H) |
| 1.09 | 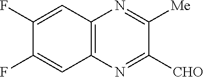 | free form | MS (APCI): m/z 209 (M + H) |
| 1.10 | 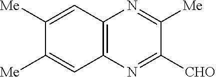 | free form | MS (APCI): m/z 201 (M + H). |
| 1.11 | 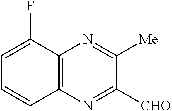 | free form | MS (APCI): m/z 191 (M + H) |
| 1.12 | 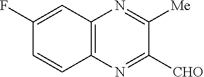 | free form | MS (APCI): m/z 191 (M + H) |
| 1.13 | 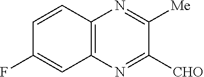 | free form | MS (APCI): m/z 191 (M + H) |
| 1.14 | 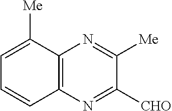 | free form | MS (APCI): m/z 187 (M + H) |
| 1.15 | 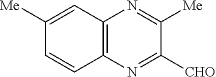 | free form | MS (APCI): m/z 187 (M + H) |
| 1.16 | 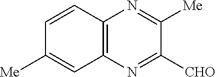 | free form | MS (APCI): m/z 187 (M + H). |
| 1.17 | 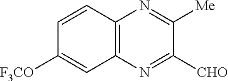 | free form | MS (APCI): m/z 257 (M + H). |
| 1.18(a) | 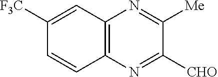 | free form | MS (APCI): m/z 273 (M + H). |
| 1.18(b) | 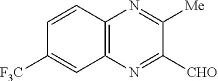 | free form | MS (APCI): m/z 273 (M + H) |
| 1.19(a) | 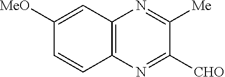 | free form | MS (APCI): m/z 203 (M + H) |
| 1.19(b) | 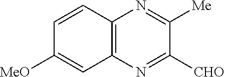 | free form | MS (APCI): m/z 203 (M + H) |
| 1.20 | 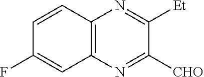 | free form | MS (APCI): m/z 205 (M + H) |
| 2.01 | 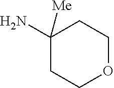 | HCl | MS (APCI): m/z 116 (M + H) |
| 2.02 | 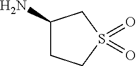 | HCl | MS (APCI): m/z 136 (M + H) |
| 2.03 | 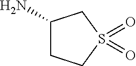 | HCl | MS (APCI): m/z 136 (M + H) |
| 2.04 | 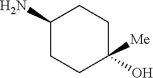 | free form | MS (APCI): m/z 130 (M + H) |
| 2.05 | 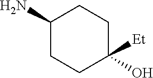 | free form | MS (APCI): m/z 144 (M + H). |
| 2.06 | 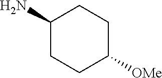 | HCl | mp 198-199° C. MS (APCI): m/z 130 (M + H) |
| 2.07 | 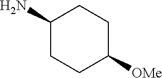 | HCl | MS (APCI): m/z 230 (M + H) |
| 2.08 | 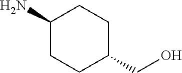 | HCl | MS (APCI): m/z 130 (M + H) |
| 2.09 | 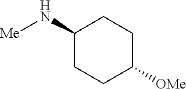 | HCl | MP 139-140° C. MS (APCI): m/z 144 (M + H). |
| 3.01 | 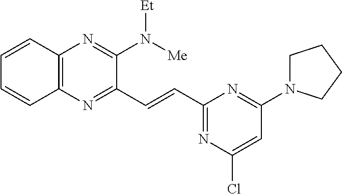 | free form | MS (APCI): m/z 395/397 (M + H) |
| 3.02 | 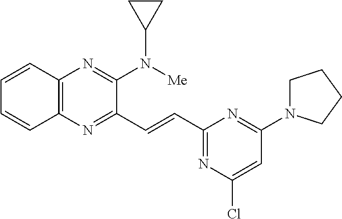 | free form | MS (APCI): m/z 407/409 (M + H) |
| 3.03 | 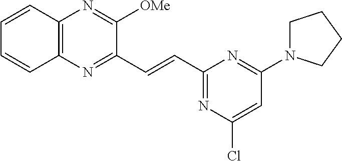 | free form | MS (APCI): m/z 368/370 (M + H) |
| 3.04 | 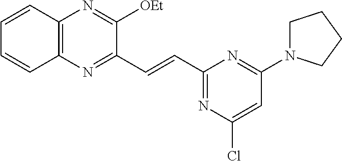 | free form | MS (APCI): m/z 382/384 (M + H). |
| 3.05 | 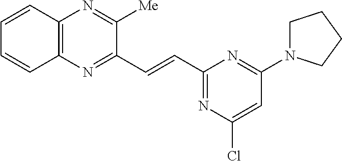 | free form | MS (APCI): m/z 352/354 (M + H) |
| 3.06 | 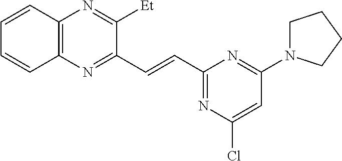 | free form | mp 211-212° C. MS (APCI): m/z 366/368 (M + H). |
| 3.07 | 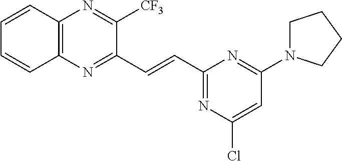 | free form | MS (APCI): m/z 406/408 (M + H) |
| 3.08 | 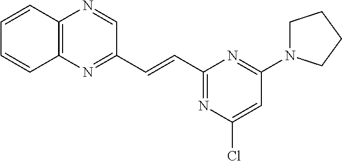 | free form | MS (APCI): m/z 338/340 (M + H) |
| 3.09 | 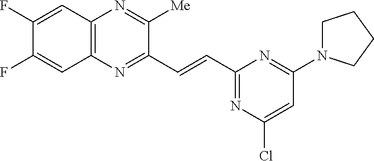 | free form | mp 226-230° C. MS (APCI): m/z 388/390 (M + H) |
| 3.10 | 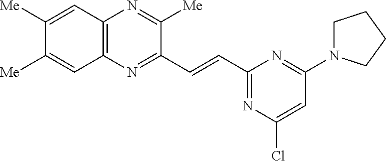 | free form | mp 206° C. MS (APCI): m/z 380/382 (M + H) |
| 3.11 | 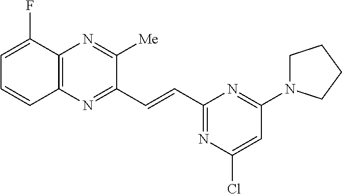 | free form | MS (APCI): m/z 370/372 (M + H) |
| 3.12 | 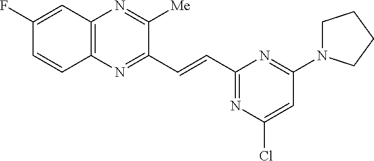 | free form | MS (APCI): m/z 370/372 (M + H) |
| 3.13 | 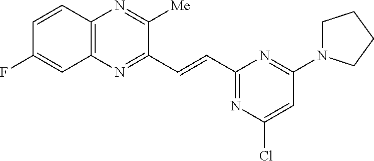 | free form | MS (APCI): m/z 370/372 (M + H) |
| 3.14 | 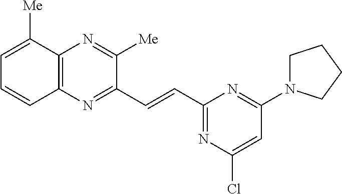 | free form | MS (APCI): m/z 366/368 |
| 3.15 | 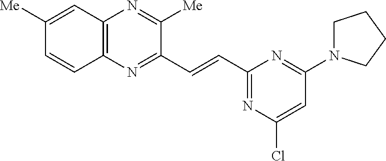 | free form | MS (APCI): m/z 366/368 (M + H) |
| 3.16 | 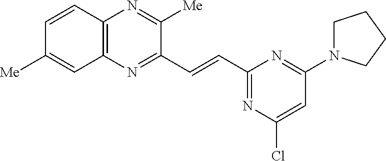 | free form | MS (APCI): m/z 366/368 (M + H) |
| 3.17 | 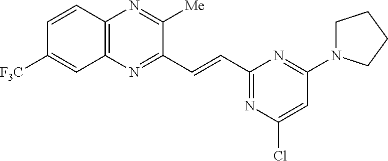 | free form | MS (APCI): m/z 420/422 (M + H). |
| 3.18 | 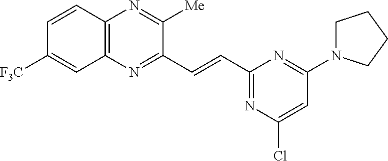 | free form | MS (APCI): m/z 420/422 (M + H). |
| 3.19 | 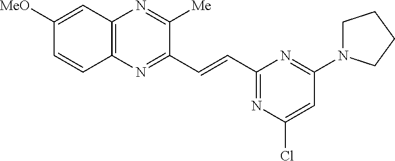 | free form | MS (APCI): m/z 382/384 (M + H). |
| 3.20 | 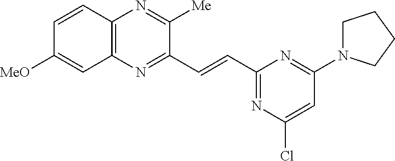 | free form | mp 191-192° C. MS (APCI): m/z 382/384 (M + H) |
| 3.21 | 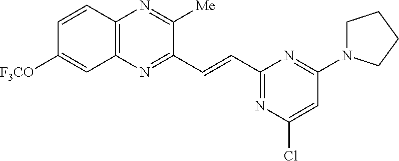 | free form | MS (APCI): m/z 436/438 (M + H) |
| 3.22 | 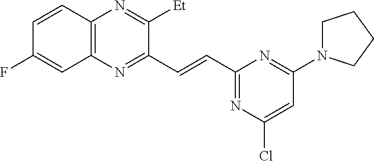 | free form | MS (APCI): m/z 384/386 (M + H) |
| 3.23 | 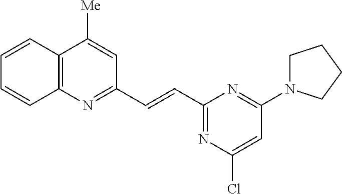 | free form | mp 212-213° C. MS (APCI): m/z 351/353 (M + H) |
| 3.24 | 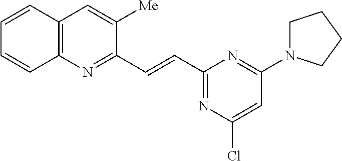 | free form | mp 236-237° C. MS (APCI): m/z 351/353 (M + H) |
| 3.25 | 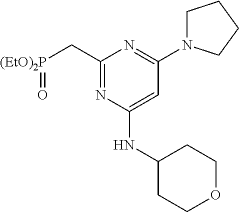 | free form | mp 122-123° C. |

1. A tri-substituted pyrimidine compound represented by formula [I]: 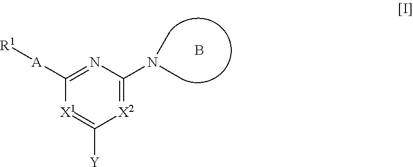
wherein:
either one of X¹ and X² is N, and the other of X¹ and X² is CH;
A is *-CH═CH—, *-C(Alk)=CH—, *-CH₂—CH₂— or *-O—CH₂— (* is a bond with R¹);
Alk is a lower alkyl group;
Ring B is an optionally substituted nitrogen-containing aliphatic heterocyclic group;
R¹ is an optionally substituted quinoxalinyl or an optionally substituted quinolyl;
Y is a substituted amino group of formula: 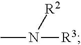
R² is a group selected from the group consisting of the following formula (1), (2) and (3); or R² and R³, together with the nitrogen atom to which they are attached, form a morpholino group, or a piperidino group substituted on the 4-position by lower alkoxy; 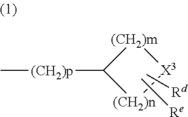
wherein:
X³ is —O—, —S— or —SO₂—;
m and n are each independently 0, 1, 2, 3 or 4, and m+n is 2, 3, 4 or 5;
p is 0, 1, 2, 3 or 4; and
R^{d} and R^{e} are the same or different and each independently are hydrogen, lower alkyl or halogen; 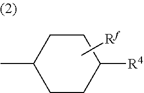
wherein:
R⁴ is a group selected from the group consisting of hydroxy, lower alkoxy, lower cycloalkyloxy, hydroxy-substituted lower alkyl, lower alkoxy-substituted lower alkyl and lower cycloalkyloxy-substituted lower alkyl; and
R^{f} is hydrogen, lower alkyl, lower cycloalkyl, or halogen; and
—(CH₂)_{q}—O—R⁵ (3)
wherein:
R⁵ is hydrogen, lower alkyl or lower cycloalkyl; and
q is 1, 2, 3 or 4;
R³ is a group selected from the group consisting of hydrogen, lower alkyl, lower cycloalkyl, lower alkoxy-substituted lower alkyl and lower cycloalkyloxy-substituted lower alkyl;
or R³ and R², together with the nitrogen atom to which they are attached, form a morpholino group, or a piperidino group substituted on the 4-position by lower alkoxy,
or a pharmaceutically acceptable salt thereof.

2. The compound of claim 1, or a pharmaceutically acceptable salt thereof wherein when A is *-CH═CH— or *-C(Alk)=CH—, the double bond in A is E isomeric form.

3. The compound of claim 1, or a pharmaceutically acceptable salt thereof wherein R¹ is a group represented by formula [X]: 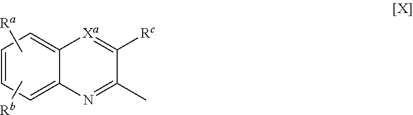
wherein:
X^{a} is N or CH;
R^{a}, R^{b} and R^{c} each independently are selected from the group consisting of hydrogen; halogen; hydroxy; lower alkyl; lower cycloalkyl; halo-lower alkyl; lower alkoxy; halo-lower alkoxy; nitro group; amino group; and amino group mono- or di-substituted by the same or different substituent(s) selected from the group consisting of lower alkyl and lower cycloalkyl.

4. The compound of claim 3, or a pharmaceutically acceptable salt thereof wherein X^{a} is N.

5. The compound of any one of claims 1 to 4, or a pharmaceutically acceptable salt thereof wherein R² is a group represented by formula:

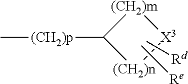

6. The compound of any one of claims 1 to 4, or a pharmaceutically acceptable salt thereof wherein R² is a group represented by formula:

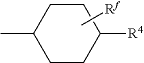

7. The compound of any one of claims 1 to 4, or a pharmaceutically acceptable salt thereof wherein A is *-CH═CH—, *-C(Alk)=CH— or *-CH₂—CH₂—.

8. The compound of any one of claims 1 to 4, or a pharmaceutically acceptable salt thereof wherein A is *-CH═CH—.

9. The compound of any one of claims 1 to 4, or a pharmaceutically acceptable salt thereof wherein X¹ is N, X² is CH, and A is *-CH═CH—.

10. The compound of any one of claims 1 to 4, or a pharmaceutically acceptable salt thereof wherein A is *-O—CH₂—.

11. A compound selected from
N,N-dimethyl-3-{(E)-2-[4-pyrrolidin-1-yl-6-(tetrahydro-2H-pyran-4-ylamino)pyrimidin-2-yl]vinyl}quinoxalin-2-amine;
3-((E)-2-{4-[(2-methoxyethyl)amino}-6-pyrrolidin-1-ylpyrimidin-2-yl]vinyl)-N,N-dimethylquinoxalin-2-amine;
3-[(E)-2-(4-[(3R)-1,1-dioxidotetrahydro-3-thienyl]amino-6-pyrrolidin-1-ylpyrimidin-2-yl)vinyl]-N,N-dimethylquinoxalin-2-amine;
N-cyclopropyl-N-methyl-3-{(E)-2-[4-pyrrolidin-1-yl-6-(tetrahydro-2H-pyran-4-ylamino)pyrimidin-2-yl]vinyl}quinoxalin-2-amine;
trans-1-methyl-4-({2-[(E)-2-(3-methylquinoxalin-2-yl)vinyl]-6-pyrrolidin-1-ylpyrimidin-4-yl}amino)cyclohexanol;
[trans-4-({2-[(E)-2-(3-methylquinoxalin-2-yl)vinyl]-6-pyrrolidin-1-ylpyrimidin-4-yl}amino)cyclohexyl]methanol;
6-pyrrolidin-1-yl-N-[(3R)-tetrahydrofuran-3-yl]-2-[(E)-2-(3,6,7-trimethylquinoxalin-2-yl)vinyl]pyrimidin-4-amine;
2-[(E)-2-(6-fluoro-3-methylquinoxalin-2-yl)vinyl]-N-(trans-4-methoxycyclohexyl)-6-pyrrolidin-1-ylpyrimidin-4-amine;
2-[(E)-2-(7-fluoro-3-methylquinoxalin-2-yl)vinyl]-6-pyrrolidin-1-yl-N-(tetrahydro-2H-pyran-4-yl)pyrimidin-4-amine;
trans-4-({2-[(E)-2-(3,7-dimethylquinoxalin-2-yl)vinyl]-6-pyrrolidin-1-ylpyrimidin-4-yl}amino)-1-methylcyclohexanol;
N-[(3R)-1,1-dioxidotetrahydro-3-thienyl]-2-{(E)-2-[3-methyl-7-(trifluoromethyl)quinoxalin-2-yl]vinyl}-6-pyrrolidin-1-ylpyrimidin-4-amine;
2-[(E)-2-(7-methoxy-3-methylquinoxalin-2-yl)vinyl]-6-pyrrolidin-1-yl-N-(tetrahydro-2H-pyran-4-yl)pyrimidin-4-amine;
trans-4-[(2-{(E)-2-[3-methyl-7-(trifluoromethoxy)quinoxalin-2-yl]vinyl}-6-pyrrolidin-1-ylpyrimidin-4-yl)amino]cyclohexanol;
2-[(E)-2-(3-methylquinolin-2-yl)vinyl]-6-pyrrolidin-1-yl-N-(tetrahydro-2H-pyran-4-yl)pyrimidin-4-amine;
N-[(3R)-1,1-dioxidotetrahydro-3-thienyl]-2-[(E)-2-(3-methylquinolin-2-yl)vinyl]-6-pyrrolidin-1-ylpyrimidin-4-amine;
3-{(E)-2-[4-pyrrolidin-1-yl-6-(tetrahydro-2H-pyran-4-ylamino)pyrimidin-2-yl]vinyl}quinoxalin-2-ol;
N,N-dimethyl-3-[(E)-2-(4-morpholin-4-yl-6-pyrrolidin-1-ylpyrimidin-2-yl)vinyl]quinoxalin-2-amine;
3-((E)-2-{4-[cyclopropyl)tetrahydro-2H-pyran-4-yl)amino]-6-pyrrolidin-1-ylpyrimidin-2-yl}vinyl)-N,N-dimethylquinoxalin-2-amine;
N-cyclopropyl-N-methyl-3-((E)-2-{4-[methyl(tetrahydro-2H-pyran-4-yl)amino]-6-pyrrolidin-1-ylpyrimidin-2-yl}vinyl)quinoxalin-2-amine;
N-(trans-4-methoxycyclohexyl)-2-{2-[3-methyl-7-(trifluoromethyl)quinoxalin-2-yl]ethyl}-6-pyrrolidin-1-ylpyrimidin-4-amine;
N-methyl-2-{[(3-methylquinoxalin-2-yl)oxy]methyl}-6-pyrrolidin-1-yl-N-(tetrahydro-2H-pyran-4-yl)pyrimidin-4-amine; and
6-{[(3-methylquinoxalin-2-yl)oxy]methyl}-2-pyrrolidin-1-yl-N-(tetrahydro-2H-pyran-4-yl)pyrimidin-4-amine;
or a pharmaceutically acceptable salt thereof.

12. A method of inhibiting a phosphodiesterase 10 activity in a patient, comprising administering to the patient an effective amount of a tri-substituted pyrimidine compound represented by formula [I⁰]: 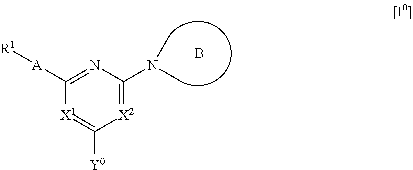
wherein:
either one of X¹ and X² is N, and the other of X¹ and X² is CH;
A is *-CH═CH—, *-C(Alk)=CH—, *-CH₂—CH₂— or *-O—CH₂— (* is a bond with R¹);
Alk is a lower alkyl group;
Ring B is an optionally substituted nitrogen-containing aliphatic heterocyclic group;
R¹ represents an optionally substituted quinoxalinyl or an optionally substituted quinolyl;
Y⁰ is mono- or di-substituted amino group,
or a pharmaceutically acceptable salt thereof.

13. The method of claim 12, for treating or preventing a disease or condition which is expected to be ameliorated by inhibition of phosphodiesterase 10 activity, by inhibiting phosphodiesterase 10 activity in the patient.

14. The method of claim 13, wherein the disease or condition which is expected to be ameliorated by inhibition of phosphodiesterase 10 activity is a disease or condition selected from the group consisting of schizophrenia, anxiety disorder, drug addiction, a disease comprising as a symptom a deficiency in cognition, mood disorder and mood episode.

15. Use of the tri-substituted pyrimidine compound represented by formula [I⁰] as set forth in claim 12 or a pharmaceutically acceptable salt thereof for the manufacture of a medicament for inhibiting phosphodiesterase 10 activity.

16. A pharmaceutical composition for inhibiting phosphodiesterase 10 activity, comprising the tri-substituted pyrimidine compound represented by formula [I⁰] as set forth in claim 12 or a pharmaceutically acceptable salt thereof as an active ingredient.
